(19) United States
(12) Patent Application Publication
(10) Pub. No.: US 2025/0154502 A1
(43) Pub. Date: May 15, 2025

(54) METHODS FOR SEAMLESS NUCLEIC ACID ASSEMBLY

(71) Applicant: Twist Bioscience Corporation, South San Francisco, CA (US)

(72) Inventors: Rebecca NUGENT, San Francisco, CA (US); Siyuan CHEN, San Mateo, CA (US); Elian LEE, Union City, CA (US); Nathan RAYNARD, San Francisco, CA (US)

(73) Assignee: Twist Bioscience Corporation, South San Francisco, CA (US)

(21) Appl. No.: 19/029,966

(22) Filed: Jan. 17, 2025

Related U.S. Application Data
(63) Continuation of application No. 17/720,210, filed on Apr. 13, 2022.
Application No. 17/720,210 is a continuation of application No. 16/879,705, filed on May 20, 2020, now Pat. No. 11,332,740.
Application No. 16/879,705 is a continuation of application No. 16/006,581, filed on Jun. 12, 2018, now Pat. No. 10,696,965.
(60) Provisional application No. 62/663,089, filed on Apr. 26, 2018.
(60) Provisional application No. 62/518,496, filed on Jun. 12, 2017.

(51) Int. Cl. C12N 15/10; C12N 9/00; C12N 9/12; C12P 19/34; C12Q 1/6811; C12Q 1/6855; C12Q 1/6869

(52) U.S. Cl. CPC C12N 15/1096; C12N 9/1252; C12N 9/93; C12N 15/1031; C12N 15/1093; C12P 19/34; C12Q 1/6811; C12Q 1/6855; C12Q 1/6869; C12Q 2531/101; C12Y 207/07007; C12Y 301/11002

(57) ABSTRACT

Provided herein are methods, systems, and compositions for seamless nucleic acid assembly. Methods, systems, and compositions as provided herein provide for efficient assembly of nucleic acids without primer removal. Methods, systems, and compositions for seamless nucleic acid assembly comprise use of an endonuclease or exonuclease, optionally in conjunction with additional enzymes to assemble nucleic acids or polynucleotides.

### CROSS-REFERENCE

This application is a continuation of U.S. patent application Ser. No. 17/720,210, filed Apr. 13, 2022, which is a continuation of U.S. patent application Ser. No. 16/879,705, filed May 20, 2020, now U.S. Pat. No. 11,332,740, issued May 17, 2022, which is a continuation application of U.S. patent application Ser. No. 16/006,581, filed Jun. 12, 2018, now U.S. Pat. No. 10,696,965, issued Jun. 30, 2020, which claims the benefit of U.S. Provisional Patent Application No. 62/663,089 filed Apr. 26, 2018; and U.S. Provisional Patent Application No. 62/518,496 filed on Jun. 12, 2017, each of which is incorporated herein by reference in its entirety.

### SEQUENCE LISTING

The instant application contains a Sequence Listing which has been submitted electronically in XML format and is hereby incorporated by reference in its entirety. The sequence listing was created on Jan. 17, 2025, is named 00415-0020_SequenceListing.xml and is 5,713 bytes in size.

### BACKGROUND

De novo nucleic acid synthesis is a powerful tool for basic biological research and biotechnology applications. While various methods are known for the synthesis of relatively short fragments of nucleic acids on a small scale, these techniques suffer from scalability, automation, speed, accuracy, and cost. Thus, a need remains for efficient methods of seamless nucleic acid assembly.

### BRIEF SUMMARY

Provided herein are methods for nucleic acid synthesis and assembly, comprising: (a) providing a plurality of polynucleotides; and (b) mixing the plurality of polynucleotides with an exonuclease, a flap endonuclease, a polymerase, and a ligase, wherein the plurality of polynucleotides are annealed in a processive predetermined order based on a complementary sequence between adjacent polynucleotides. Further provided herein are methods for nucleic acid synthesis and assembly, wherein the exonuclease is exonuclease III. Further provided herein are methods for nucleic acid synthesis and assembly, wherein the flap endonuclease is flap endonuclease 1, exonuclease 1, XPG, Dna2, or GEN1. Further provided herein are methods for nucleic acid synthesis and assembly, wherein the polymerase comprises 5′ to 3′ polymerase activity. Further provided herein are methods for nucleic acid synthesis and assembly, wherein the polymerase is a DNA polymerase. Further provided herein are methods for nucleic acid synthesis and assembly, wherein the ligase catalyzes joining of at least two nucleic acids. Further provided herein are methods for nucleic acid synthesis and assembly, wherein a concentration of the flap endonuclease 1 is in a range of about 0.32 U to about 4.8 U. Further provided herein are methods for nucleic acid synthesis and assembly, wherein a concentration of the exonuclease III is in a range of about 0.1 U to about 10 U. Further provided herein are methods for nucleic acid synthesis and assembly, wherein a concentration of the exonuclease III is in a range of about 0.5 U to about 1.0 U. Further provided herein are methods for nucleic acid synthesis and assembly, wherein a concentration of the exonuclease III is in a range of about 1.0 U to about 2.0 U. Further provided herein are methods for nucleic acid synthesis and assembly, wherein a concentration of the polymerase is in a range of about 0.1 U to about 2 U. Further provided herein are methods for nucleic acid synthesis and assembly, wherein a concentration of the polymerase is about 0.1 U. Further provided herein are methods for nucleic acid synthesis and assembly, wherein a concentration of the polymerase is about 0.2 U. Further provided herein are methods for nucleic acid synthesis and assembly, wherein a concentration of the ligase is up to about 2.0 U. Further provided herein are methods for nucleic acid synthesis and assembly, wherein a concentration of the ligase is in a range of about 4.0 U to about 8.0 U.

Provided herein are methods for nucleic acid synthesis and assembly, comprising: (a) providing a first double stranded nucleic acid; (b) providing a second double stranded nucleic acid; (c) providing a third double stranded nucleic acid comprising in 5′ to 3′ order: a 5′ flanking adapter sequence, a first homology sequence to the first double stranded nucleic acid, a second homology sequence to the second double stranded nucleic acid, and a 3′ flanking adapter sequence; and (d) mixing the first double stranded nucleic acid, the second double stranded nucleic acid, and the third double stranded nucleic acid with a reaction mixture comprising an exonuclease, a flap endonuclease, a polymerase, and a ligase. Further provided herein are methods for nucleic acid synthesis and assembly, wherein the exonuclease is exonuclease III. Further provided herein are methods for nucleic acid synthesis and assembly, wherein the flap endonuclease is flap endonuclease 1, exonuclease 1, XPG, Dna2, or GEN1. Further provided herein are methods for nucleic acid synthesis and assembly, wherein an amount of the flap endonuclease 1 provided is about 0.32 U to about 4.8 U. Further provided herein are methods for nucleic acid synthesis and assembly, wherein an amount of the flap endonuclease 1 provided is less than about 5.0 U. Further provided herein are methods for nucleic acid synthesis and assembly, wherein the polymerase comprises 5′ to 3′ polymerase activity. Further provided herein are methods for nucleic acid synthesis and assembly, wherein the polymerase is a DNA polymerase. Further provided herein are methods for nucleic acid synthesis and assembly, wherein the ligase catalyzes joining of at least two nucleic acids. Further provided herein are methods for nucleic acid synthesis and assembly, wherein the first homology sequence or the second homology sequence is about 10 to about 100 base pairs. Further provided herein are methods for nucleic acid synthesis and assembly, wherein the first homology sequence and the second homology sequence are each independently about 10 to about 100 base pairs. Further provided herein are methods for nucleic acid synthesis and assembly, wherein the first homology sequence or the second homology sequence is about 20 to about 80 base pairs. Further provided herein are methods for nucleic acid synthesis and assembly, wherein the first homology sequence and the second homology sequence are each independently about 20 to about 80 base pairs. Further provided herein are methods for nucleic acid synthesis and assembly, wherein the first homology sequence or the second homology sequence is about 40 base pairs. Further provided herein are methods for nucleic acid synthesis and assembly, wherein the first homology sequence and the second homology sequence are each independently about 40 base pairs. Further provided herein are methods for nucleic acid synthesis and assembly, wherein a concentration of the exonuclease III is in a range of about 0.1 U to about 10 U. Further provided herein are methods for nucleic acid synthesis and assembly, wherein a concentration of the exonuclease III is in a range of about 0.5 U to about 1.0 U. Further provided herein are methods for nucleic acid synthesis and assembly, wherein a concentration of the exonuclease III is in a range of about 1.0 U to about 2.0 U. Further provided herein are methods for nucleic acid synthesis and assembly, wherein a concentration of the polymerase is present in an amount of about 0.1 U to about 2 U. Further provided herein are methods for nucleic acid synthesis and assembly, wherein a concentration of the polymerase is about 0.1 U to about 0.2 U. Further provided herein are methods for nucleic acid synthesis and assembly, wherein a concentration of the ligase is up to about 2.0 U. Further provided herein are methods for nucleic acid synthesis and assembly, wherein a concentration of the ligase is in a range of about 4.0 U to about 8.0 U. Further provided herein are methods for nucleic acid synthesis and assembly, wherein a concentration of the ligase is in a range of about 0.5 U to about 1.0 U. Further provided herein are methods for nucleic acid synthesis and assembly, wherein the first double stranded nucleic acid, the second double stranded nucleic acid, or the third double stranded nucleic acid or any combination thereof is a linear fragment. Further provided herein are methods for nucleic acid synthesis and assembly, wherein a product following step (d) is a linear fragment. Further provided herein are methods for nucleic acid synthesis and assembly, wherein a product following step (d) is a circular fragment.

Provided herein are methods for nucleic acid assembly, comprising: (a) providing a first double stranded nucleic acid comprising in 5′ to 3′ order: a 5′ flanking adapter sequence, a homology sequence, an insert sequence, and a 3′ flanking adapter sequence; (b) providing a second double stranded nucleic acid comprising in 5′ to 3′ order: a 5′ flanking adapter sequence, a homology sequence, an insert sequence, and a 3′ flanking adapter sequence; and (c) mixing the first double stranded nucleic acid and the second double stranded nucleic acid with a reaction mixture comprising an exonuclease, a flap endonuclease, a polymerase, and a ligase. Further provided herein are methods for nucleic acid assembly, wherein the exonuclease is exonuclease III. Further provided herein are methods for nucleic acid assembly, wherein the flap endonuclease is flap endonuclease 1, exonuclease 1, XPG, Dna2, or GEN1. Further provided herein are methods for nucleic acid assembly, wherein an amount of the flap endonuclease 1provided is about 0.32 U to about 4.8 U. Further provided herein are methods for nucleic acid assembly, wherein an amount of the flap endonuclease 1 provided is less than about 5.0 U. Further provided herein are methods for nucleic acid assembly, wherein the polymerase comprises 5′ to 3′ polymerase activity. Further provided herein are methods for nucleic acid assembly, wherein the polymerase is a DNA polymerase. Further provided herein are methods for nucleic acid assembly, wherein the ligase catalyzes joining of at least two nucleic acids. Further provided herein are methods for nucleic acid assembly, wherein the homology sequence of the first double stranded nucleic acid or the homology sequence of the second double stranded nucleic acid is about 10 to about 100 base pairs. Further provided herein are methods for nucleic acid assembly, wherein the homology sequence of the first double stranded nucleic acid and the homology sequence of the second double stranded nucleic acid are each independently about 10 to about 100 base pairs. Further provided herein are methods for nucleic acid assembly, wherein the homology sequence of the first double stranded nucleic acid or the homology sequence of the second double stranded nucleic acid is about 20 to about 80 base pairs. Further provided herein are methods for nucleic acid assembly, wherein the homology sequence of the first double stranded nucleic acid and the homology sequence of the second double stranded nucleic acid are each independently about 20 to about 80 base pairs. Further provided herein are methods for nucleic acid assembly, wherein the homology sequence of the first double stranded nucleic acid or the homology sequence of the second double stranded nucleic acid is about 40 base pairs. Further provided herein are methods for nucleic acid assembly, wherein the homology sequence of the first double stranded nucleic acid and the homology sequence of the second double stranded nucleic acid are each independently about 40 base pairs. Further provided herein are methods for nucleic acid assembly, wherein a concentration of the exonuclease III is in a range of about 0.1 U to about 10 U. Further provided herein are methods for nucleic acid assembly, wherein a concentration of the exonuclease III is in a range of about 0.5 U to about 1.0 U. Further provided herein are methods for nucleic acid assembly, wherein a concentration of the exonuclease III is in a range of about 1.0 U to about 2.0 U. Further provided herein are methods for nucleic acid assembly, wherein a concentration of the polymerase is present in an amount of about 0.1 U to about 2 U. Further provided herein are methods for nucleic acid assembly, wherein a concentration of the polymerase is about 0.1 U to about 0.2 U. Further provided herein are methods for nucleic acid assembly, wherein a concentration of the ligase is up to about 2.0 U. Further provided herein are methods for nucleic acid assembly, wherein a concentration of the ligase is in a range of about 4.0 U to about 8.0 U. Further provided herein are methods for nucleic acid assembly, wherein a concentration of the ligase is in a range of about 0.5 U to about 1.0 U. Further provided herein are methods for nucleic acid assembly, wherein the first double stranded nucleic acid or the second double stranded nucleic acid is a linear fragment. Further provided herein are methods for nucleic acid assembly, wherein a product following step (c) is a linear fragment. Further provided herein are methods for nucleic acid assembly, wherein a product following step (c) is a circular fragment.

Provided herein are methods for nucleic acid assembly, comprising: (a) providing a first double stranded nucleic acid comprising in 5′ to 3′ order: a 5′ flanking adapter sequence, a homology sequence, an insert sequence, and a 3′ flanking adapter sequence; (b) providing a second double stranded nucleic acid comprising in 5′ to 3′ order: a 5′ flanking adapter sequence, a homology sequence, an insert sequence, and a 3′ flanking adapter sequence; and (c) mixing the first double stranded nucleic acid and the second double stranded nucleic acid with a reaction mixture comprising an exonuclease, a flap endonuclease, a polymerase, and a ligase at a temperature of about 30° C. to about 60° C. Further provided herein are methods for nucleic acid assembly, wherein the exonuclease is exonuclease III. Further provided herein are methods for nucleic acid assembly, wherein the flap endonuclease is flap endonuclease 1, exonuclease 1, XPG, Dna2, or GEN1. Further provided herein are methods for nucleic acid assembly, wherein a concentration of the flap endonuclease 1 is in a range of about 0.32 U to about 4.8 U. Further provided herein are methods for nucleic acid assembly, wherein the polymerase comprises 5′ to 3′ polymerase activity. Further provided herein are methods for nucleic acid assembly, wherein the polymerase is a DNA polymerase. Further provided herein are methods for nucleic acid assembly, wherein the ligase catalyzes joining of at least two nucleic acids. Further provided herein are methods for nucleic acid assembly, wherein the homology sequence of the first double stranded nucleic acid or the homology sequence of the second double stranded nucleic acid is about 10 to about 100 base pairs. Further provided herein are methods for nucleic acid assembly, wherein the homology sequence of the first double stranded nucleic acid and the homology sequence of the second double stranded nucleic acid are each independently about 10 to about 100 base pairs. Further provided herein are methods for nucleic acid assembly, wherein the homology sequence of the first double stranded nucleic acid or the homology sequence of the second double stranded nucleic acid is about 20 to about 80 base pairs. Further provided herein are methods for nucleic acid assembly, wherein the homology sequence of the first double stranded nucleic acid and the homology sequence of the second double stranded nucleic acid are each independently about 20 to about 80 base pairs. Further provided herein are methods for nucleic acid assembly, wherein the homology sequence of the first double stranded nucleic acid or the homology sequence of the second double stranded nucleic acid is about 40 base pairs. Further provided herein are methods for nucleic acid assembly, wherein the homology sequence of the first double stranded nucleic acid and the homology sequence of the second double stranded nucleic acid are each independently about 40 base pairs. Further provided herein are methods for nucleic acid assembly, wherein a concentration of the exonuclease III is in a range of about 0.1 U to about 10 U. Further provided herein are methods for nucleic acid assembly, wherein a concentration of the exonuclease III is in a range of about 0.5 U to about 1.0 U. Further provided herein are methods for nucleic acid assembly, wherein a concentration of the exonuclease III is in a range of about 1.0 U to about 2.0 U. Further provided herein are methods for nucleic acid assembly, wherein a concentration of the polymerase is in a range of about 0.1 U to about 2 U. Further provided herein are methods for nucleic acid assembly, wherein a concentration of the polymerase is about 0.1 U. Further provided herein are methods for nucleic acid assembly, wherein a concentration of the polymerase is about 0.2 U. Further provided herein are methods for nucleic acid assembly, wherein a concentration of the ligase is up to about 2.0 U. Further provided herein are methods for nucleic acid assembly, wherein a concentration of the ligase is in a range of about 4.0 U to about 8.0 U. Further provided herein are methods for nucleic acid assembly, wherein a concentration of the ligase is in a range of 0.5 U to about 1.0 U. Further provided herein are methods for nucleic acid assembly, wherein the first double stranded nucleic acid or the second double stranded nucleic acid is a linear fragment. Further provided herein are methods for nucleic acid assembly, wherein a product following step (c) is a linear fragment. Further provided herein are methods for nucleic acid assembly, wherein a product following step (c) is a circular fragment.

Provided herein are methods for nucleic acid assembly, comprising: (a) providing a first double stranded nucleic acid comprising in 5′ to 3′ order: a 5′ flanking adapter, a homology sequence, an insert sequence, and a 3′ flanking adapter sequence; (b) providing a second double stranded nucleic acid comprising in 5′ to 3′ order: a 5′ flanking adapter, a homology sequence, an insert sequence, and a 3′ flanking adapter sequence; and (c) mixing the first double stranded nucleic acid and the second double stranded nucleic acid with a reaction mixture comprising, a flap endonuclease, wherein the flap endonuclease results in a 5′ overhang; a polymerase; and a ligase.

Provided herein are methods for nucleic acid assembly, comprising: (a) providing a first double stranded nucleic acid comprising in 5′ to 3′ order: a 5′ flanking adapter sequence, a homology sequence, an insert sequence, and a 3′ flanking adapter sequence; (b) providing a second double stranded nucleic acid comprising in 5′ to 3′ order: a 5′ flanking adapter sequence, a homology sequence, an insert sequence, and a 3′ flanking adapter sequence; and (c) mixing the first double stranded nucleic acid and the second double stranded nucleic acid with a reaction mixture comprising about 0.5 U to about 1.0 U of an exonuclease, about 0.32 U to about 4.8 U of a flap endonuclease, about 0.1 U to about 2 U of a polymerase, and up to about 2.0 U of a ligase.

Provided herein are methods for nucleic acid assembly, comprising: (a) providing a first double stranded nucleic acid comprising in 5′ to 3′ order: a 5′ flanking adapter sequence, a homology sequence, an insert sequence, and a 3′ flanking adapter sequence; (b) providing a second double stranded nucleic acid comprising in 5′ to 3′ order: a 5′ flanking adapter sequence, a homology sequence, an insert sequence, and a 3′ flanking adapter sequence; and (c) mixing the first double stranded nucleic acid and the second double stranded nucleic acid with a reaction mixture comprising about 0.32 U to about 4.8 U of a flap endonuclease, about 0.1 U to about 2 U of a polymerase, and up to about 2.0 U of a ligase.

Provided herein are methods for nucleic acid assembly, comprising: (a) providing a plurality of polynucleotides, wherein each of the polynucleotides do not comprise a terminal region of sequence homology to another polynucleotide of the plurality of polynucleotides; and (b) mixing the plurality of polynucleotides with an exonuclease, an endonuclease, a polymerase, and a ligase, wherein the plurality of polynucleotides are annealed in a processive predetermined order based on a complementary sequence between adjacent polynucleotides. Further provided herein are methods for nucleic acid assembly, wherein the exonuclease is exonuclease III. Further provided herein are methods for nucleic acid assembly, wherein the endonuclease is a flap endonuclease. Further provided herein are methods for nucleic acid assembly, wherein the flap endonuclease is flap endonuclease 1, exonuclease 1, XPG, Dna2, or GEN1. Further provided herein are methods for nucleic acid assembly, wherein a concentration of the flap endonuclease 1 is in a range of about 0.32 U to about 4.8 U. Further provided herein are methods for nucleic acid assembly, wherein the polymerase comprises 5′ to 3′ polymerase activity. Further provided herein are methods for nucleic acid assembly, wherein the polymerase is a DNA polymerase. Further provided herein are methods for nucleic acid assembly, wherein the ligase catalyzes joining of at least two nucleic acids. Further provided herein are methods for nucleic acid assembly, wherein a concentration of the exonuclease III is in a range of about 0.1 U to about 10 U. Further provided herein are methods for nucleic acid assembly, wherein a concentration of the polymerase is in a range of about 0.01 U to about 2 U. Further provided herein are methods for nucleic acid assembly, wherein a concentration of the polymerase is about 0.1 U. Further provided herein are methods for nucleic acid assembly, wherein a concentration of the polymerase is about 0.01 U. Further provided herein are methods for nucleic acid assembly, wherein a concentration of the ligase is up to about 2.0 U.

Provided herein are methods for nucleic acid assembly, comprising: (a) providing a first double stranded nucleic acid; (b) providing a second double stranded nucleic acid; (c) providing a third double stranded nucleic acid comprising in 5′ to 3′ order: a 5′ flanking adapter sequence, a first homology sequence to the first double stranded nucleic acid, a second homology sequence to the second double stranded nucleic acid, and a 3′ flanking adapter sequence, wherein the first double stranded nucleic acid, the second double stranded nucleic acid, and the third double stranded nucleic acid comprise non-homologous sequences at terminal regions; and (d) mixing the first double stranded nucleic acid, the second double stranded nucleic acid, and the third double stranded nucleic acid with a reaction mixture comprising an exonuclease, an endonuclease, a polymerase, and a ligase. Further provided herein are methods for nucleic acid assembly, wherein the exonuclease is exonuclease III. Further provided herein are methods for nucleic acid assembly, wherein the endonuclease is a flap endonuclease. Further provided herein are methods for nucleic acid assembly, wherein the flap endonuclease is flap endonuclease 1, exonuclease 1, XPG, Dna2, or GEN1. Further provided herein are methods for nucleic acid assembly, wherein a concentration of the flap endonuclease 1 provided is about 0.32 U to about 4.8 U. Further provided herein are methods for nucleic acid assembly, wherein the endonuclease is flap endonuclease 1 provided in a concentration less than about 5.0 U. Further provided herein are methods for nucleic acid assembly, wherein the polymerase comprises 5′ to 3′ polymerase activity. Further provided herein are methods for nucleic acid assembly, wherein the polymerase is a DNA polymerase. Further provided herein are methods for nucleic acid assembly, wherein the ligase catalyzes joining of at least two nucleic acids. Further provided herein are methods for nucleic acid assembly, wherein the homology sequence of the first double stranded nucleic acid or the homology sequence of the second double stranded nucleic acid is about 10 to about 100 base pairs. Further provided herein are methods for nucleic acid assembly, wherein the homology sequence of the first double stranded nucleic acid and the homology sequence of the second double stranded nucleic acid are each independently about 10 to about 100 base pairs. Further provided herein are methods for nucleic acid assembly, wherein the homology sequence of the first double stranded nucleic acid or the homology sequence of the second double stranded nucleic acid is about 20 to about 80 base pairs. Further provided herein are methods for nucleic acid assembly, wherein the homology sequence of the first double stranded nucleic acid and the homology sequence of the second double stranded nucleic acid are each independently about 20 to about 80 base pairs. Further provided herein are methods for nucleic acid assembly, wherein the homology sequence of the first double stranded nucleic acid or the homology sequence of the second double stranded nucleic acid is about 40 base pairs. Further provided herein are methods for nucleic acid assembly, wherein the homology sequence of the first double stranded nucleic acid and the homology sequence of the second double stranded nucleic acid are each independently about 40 base pairs. Further provided herein are methods for nucleic acid assembly, wherein a concentration of the exonuclease III is in a range of about 0.1 U to about 10 U. Further provided herein are methods for nucleic acid assembly, wherein a concentration of the polymerase is in a range of about 0.01 U to about 2 U. Further provided herein are methods for nucleic acid assembly, wherein a concentration of the ligase is up to about 2.0 U. Further provided herein are methods for nucleic acid assembly, wherein the first double stranded nucleic acid, the second double stranded nucleic acid, or the third double stranded nucleic acid, or any combination thereof is a linear fragment. Further provided herein are methods for nucleic acid assembly, wherein a product following step (d) is a linear fragment. Further provided herein are methods for nucleic acid assembly, wherein a product following step (d) is a circular fragment.

Provided herein are methods for nucleic acid assembly, comprising: (a) providing a first double stranded nucleic acid comprising in 5′ to 3′ order: a 5′ flanking adapter sequence, a homology sequence, an insert sequence, and a 3′ flanking adapter sequence; (b) providing a second double stranded nucleic acid comprising in 5′ to 3′ order: a 5′ flanking adapter sequence, a homology sequence, an insert sequence, and a 3′ flanking adapter sequence; and (c) mixing the first double stranded nucleic acid and the second double stranded nucleic acid with a reaction mixture comprising an exonuclease, an endonuclease, a polymerase, and a ligase. Further provided herein are methods for nucleic acid assembly, wherein the exonuclease is exonuclease III. Further provided herein are methods for nucleic acid assembly, wherein the endonuclease is a flap endonuclease. Further provided herein are methods for nucleic acid assembly, wherein the flap endonuclease is flap endonuclease 1, exonuclease 1, XPG, Dna2, or GEN1. Further provided herein are methods for nucleic acid assembly, wherein a concentration of the flap endonuclease 1 provided is about 0.32 U to about 4.8 U. Further provided herein are methods for nucleic acid assembly, wherein a concentration of the flap endonuclease 1 provided is less than about 5.0 U. Further provided herein are methods for nucleic acid assembly, wherein the polymerase comprises 5′ to 3′ polymerase activity. Further provided herein are methods for nucleic acid assembly, wherein the polymerase is a DNA polymerase. Further provided herein are methods for nucleic acid assembly, wherein the ligase catalyzes joining of at least two nucleic acids. Further provided herein are methods for nucleic acid assembly, wherein the homology sequence of the first double stranded nucleic acid or the homology sequence of the second double stranded nucleic acid is about 10 to about 100 base pairs. Further provided herein are methods for nucleic acid assembly, wherein the homology sequence of the first double stranded nucleic acid and the homology sequence of the second double stranded nucleic acid are each independently about 10 to about 100 base pairs. Further provided herein are methods for nucleic acid assembly, wherein the homology sequence of the first double stranded nucleic acid or the homology sequence of the second double stranded nucleic acid is about 20 to about 80 base pairs. Further provided herein are methods for nucleic acid assembly, wherein the homology sequence of the first double stranded nucleic acid and the homology sequence of the second double stranded nucleic acid are each independently about 20 to about 80 base pairs. Further provided herein are methods for nucleic acid assembly, wherein the homology sequence of the first double stranded nucleic acid or the homology sequence of the second double stranded nucleic acid is about 40 base pairs. Further provided herein are methods for nucleic acid assembly, wherein the homology sequence of the first double stranded nucleic acid and the homology sequence of the second double stranded nucleic acid are each independently about 40 base pairs. Further provided herein are methods for nucleic acid assembly, wherein a concentration of the exonuclease III is in a range of about 0.1 U to about 10 U. Further provided herein are methods for nucleic acid assembly, wherein a concentration of the polymerase is in a range of about 0.1 U to about 2 U. Further provided herein are methods for nucleic acid assembly, wherein a concentration of the polymerase is about 0.01 U to about 0.2 U. Further provided herein are methods for nucleic acid assembly, wherein a concentration of the ligase is up to about 2.0 U. Further provided herein are methods for nucleic acid assembly, wherein the first double stranded nucleic acid or the second double stranded nucleic acid or any combination thereof is a linear fragment. Further provided herein are methods for nucleic acid assembly, wherein a product following step (c) is a linear fragment. Further provided herein are methods for nucleic acid assembly, wherein a product following step (c) is a circular fragment.

Provided herein are methods for nucleic acid assembly, comprising: (a) providing a first double stranded nucleic acid comprising in 5′ to 3′ order: a 5′ flanking adapter sequence, a homology sequence, an insert sequence, and a 3′ flanking adapter sequence; (b) providing a second double stranded nucleic acid comprising in 5′ to 3′ order: a 5′ flanking adapter sequence, a homology sequence, an insert sequence, and a 3′ flanking adapter sequence; and (c) mixing the first double stranded nucleic acid and the second double stranded nucleic acid with a reaction mixture comprising an exonuclease, an endonuclease, a polymerase, and a ligase at a temperature of about 30° C. to about 60° C. Further provided herein are methods for nucleic acid assembly, wherein the exonuclease is exonuclease III. Further provided herein are methods for nucleic acid assembly, wherein the endonuclease is a flap endonuclease. Further provided herein are methods for nucleic acid assembly, wherein the flap endonuclease is flap endonuclease 1, exonuclease 1, XPG, Dna2, or GEN1. Further provided herein are methods for nucleic acid assembly, wherein a concentration of the flap endonuclease 1 is in a range of about 0.32 U to about 4.8 U. Further provided herein are methods for nucleic acid assembly, wherein the polymerase comprises 5′ to 3′ polymerase activity. Further provided herein are methods for nucleic acid assembly, wherein the polymerase is a DNA polymerase. Further provided herein are methods for nucleic acid assembly, wherein the ligase catalyzes joining of at least two nucleic acids. Further provided herein are methods for nucleic acid assembly, wherein the homology sequence of the first double stranded nucleic acid or the homology sequence of the second double stranded nucleic acid is about 10 to about 100 base pairs. Further provided herein are methods for nucleic acid assembly, wherein the homology sequence of the first double stranded nucleic acid and the homology sequence of the second double stranded nucleic acid are each independently about 10 to about 100 base pairs. Further provided herein are methods for nucleic acid assembly, wherein the homology sequence of the first double stranded nucleic acid or the homology sequence of the second double stranded nucleic acid is about 20 to about 80 base pairs. Further provided herein are methods for nucleic acid assembly, wherein the homology sequence of the first double stranded nucleic acid and the homology sequence of the second double stranded nucleic acid are each independently about 20 to about 80 base pairs. Further provided herein are methods for nucleic acid assembly, wherein the homology sequence of the first double stranded nucleic acid or the homology sequence of the second double stranded nucleic acid is about 40 base pairs. Further provided herein are methods for nucleic acid assembly, wherein the homology sequence of the first double stranded nucleic acid and the homology sequence of the second double stranded nucleic acid are each independently about 40 base pairs. Further provided herein are methods for nucleic acid assembly, wherein a concentration of the exonuclease III is in a range of about 0.1 U to about 10 U. Further provided herein are methods for nucleic acid assembly, wherein a concentration of the polymerase is in a range of about 0.01 U to about 2 U. Further provided herein are methods for nucleic acid assembly, wherein a concentration of the polymerase is about 0.1 U. Further provided herein are methods for nucleic acid assembly, wherein a concentration of the polymerase is about 0.01 U. Further provided herein are methods for nucleic acid assembly, wherein a concentration of the ligase is up to about 2.0 U. Further provided herein are methods for nucleic acid assembly, wherein the first double stranded nucleic acid or the second double stranded nucleic acid or any combination thereof is a linear fragment. Further provided herein are methods for nucleic acid assembly, wherein a product following step (c) is a linear fragment. Further provided herein are methods for nucleic acid assembly, wherein a product following step (c) is a circular fragment.

Provided herein are methods for nucleic acid assembly, comprising: (a) providing a first double stranded nucleic acid comprising in 5′ to 3′ order: a 5′ flanking adapter, a homology sequence, an insert sequence, and a 3′ flanking adapter sequence; (b) providing a second double stranded nucleic acid comprising in 5′ to 3′ order: a 5′ flanking adapter, a homology sequence, an insert sequence, and a 3′ flanking adapter sequence; and (c) mixing the first double stranded nucleic acid and the second double stranded nucleic acid with a reaction mixture comprising, an endonuclease, wherein the endonuclease results in a 5′ overhang; a polymerase; and a ligase.

Provided herein are methods for nucleic acid assembly, comprising: (a) providing a first double stranded nucleic acid comprising in 5′ to 3′ order: a 5′ flanking adapter sequence, a homology sequence, an insert sequence, and a 3′ flanking adapter sequence; (b) providing a second double stranded nucleic acid comprising in 5′ to 3′ order: a 5′ flanking adapter sequence, a homology sequence, an insert sequence, and a 3′ flanking adapter sequence; and (c) mixing the first double stranded nucleic acid and the second double stranded nucleic acid with a reaction mixture comprising about 0.5 U to about 1.0 U of an exonuclease, about 0.32 U to about 4.8 U of an endonuclease, about 0.01 U to about 2 U of a polymerase, and up to about 2.0 U of a ligase.

Provided herein are methods for nucleic acid assembly, comprising: (a) providing a first double stranded nucleic acid comprising in 5′ to 3′ order: a 5′ flanking adapter sequence, a homology sequence, an insert sequence, and a 3′ flanking adapter sequence; (b) providing a second double stranded nucleic acid comprising in 5′ to 3′ order: a 5′ flanking adapter sequence, a homology sequence, an insert sequence, and a 3′ flanking adapter sequence; and (c) mixing the first double stranded nucleic acid and the second double stranded nucleic acid with a reaction mixture comprising about 0.32 U to about 4.8 U of an endonuclease, about 0.01 U to about 2 U of a polymerase, and up to about 2.0 U of a ligase.

Provided herein are methods for nucleic acid assembly, comprising: (a) providing a first double stranded nucleic acid comprising in 5′ to 3′ order: a 5′ flanking adapter sequence, a homology sequence, an insert sequence, and a 3′ flanking adapter sequence; (b) providing a second double stranded nucleic acid comprising in 5′ to 3′ order: a 5′ flanking adapter sequence, a homology sequence, an insert sequence, and a 3′ flanking adapter sequence; and (c) mixing the first double stranded nucleic acid and the second double stranded nucleic acid with a reaction mixture comprising at least one enzyme comprising 3′ or 5′ exonuclease activity, a polymerase, and a ligase, wherein the at least one enzyme comprising 3′ or 5′ exonuclease activity removes a 5′ flanking adapter sequence or 3′ flanking adapter sequence. Further provided herein are methods for nucleic acid assembly, wherein the at least one enzyme comprising 3′ or 5′ exonuclease activity is exonuclease III. Further provided herein are methods for nucleic acid assembly, wherein the polymerase comprises 5′ to 3′ polymerase activity. Further provided herein are methods for nucleic acid assembly, wherein the polymerase is a DNA polymerase. Further provided herein are methods for nucleic acid assembly, wherein the ligase catalyzes joining of at least two nucleic acids. Further provided herein are methods for nucleic acid assembly, wherein the homology sequence of the first double stranded nucleic acid or the homology sequence of the second double stranded nucleic acid is about 40 base pairs. Further provided herein are methods for nucleic acid assembly, wherein the homology sequence of the first double stranded nucleic acid and the homology sequence of the second double stranded nucleic acid are each independently about 40 base pairs. Further provided herein are methods for nucleic acid assembly, wherein the homology sequence of the first double stranded nucleic acid or the homology sequence of the second double stranded nucleic acid is about 10 to about 100 base pairs. Further provided herein are methods for nucleic acid assembly, wherein the homology sequence of the first double stranded nucleic acid and the homology sequence of the second double stranded nucleic acid are each independently about 10 to about 100 base pairs. Further provided herein are methods for nucleic acid assembly, wherein the homology sequence of the first double stranded nucleic acid or the homology sequence of the second double stranded nucleic acid is about 20 to about 80 base pairs. Further provided herein are methods for nucleic acid assembly, wherein the homology sequence of the first double stranded nucleic acid and the homology sequence of the second double stranded nucleic acid are each independently about 20 to about 80 base pairs. Further provided herein are methods for nucleic acid assembly, wherein a concentration of the exonuclease III is in a range of about 0.1 U to about 10 U.

Provided herein are methods for nucleic acid assembly, comprising: (a) providing at least 10 different fragments, wherein each of the at least 10 different fragments do not comprise a terminal region of sequence homology to another fragment of the at least 10 different fragments; and (b) mixing the at least 10 different fragments with a plurality of enzymes, wherein the plurality of enzymes is selected from an endonuclease, an exonuclease, a polymerase, and a ligase to form a nucleic acid. Further provided herein are methods for nucleic acid assembly, wherein the nucleic acid is attached to a vector sequence. Further provided herein are methods for nucleic acid assembly, wherein the nucleic acid is 50 bases to 200 bases in length. Further provided herein are methods for nucleic acid assembly, wherein the nucleic acid is 100 bases to 2000 bases in length. Further provided herein are methods for nucleic acid assembly, wherein the exonuclease is exonuclease III. Further provided herein are methods for nucleic acid assembly, wherein the endonuclease is a flap endonuclease. Further provided herein are methods for nucleic acid assembly, wherein the flap endonuclease is flap endonuclease 1, exonuclease 1, XPG, Dna2, or GEN1. Further provided herein are methods for nucleic acid assembly, wherein the polymerase comprises 5′ to 3′ polymerase activity. Further provided herein are methods for nucleic acid assembly, wherein the polymerase is a DNA polymerase. Further provided herein are methods for nucleic acid assembly, wherein the ligase catalyzes joining of at least two nucleic acids.

Provided herein are methods for nucleic acid assembly, comprising: (a) providing a plurality of polynucleotides, wherein each of the polynucleotides do not comprise a terminal region of sequence homology to another polynucleotide of the plurality of polynucleotides; and (b) mixing the plurality of polynucleotides with a 3′ to 5′ exonuclease, a thermostable endonuclease, a high fidelity polymerase, and a thermostable ligase, wherein the plurality of polynucleotides are annealed in a processive predetermined order based on a complementary sequence between adjacent polynucleotides. Further provided herein are methods for nucleic acid assembly, wherein the exonuclease is exonuclease III. Further provided herein are methods for nucleic acid assembly, wherein the endonuclease is a flap endonuclease. Further provided herein are methods for nucleic acid assembly, wherein the flap endonuclease is flap endonuclease 1, exonuclease 1, XPG, Dna2, or GEN1. Further provided herein are methods for nucleic acid assembly, wherein the endonuclease is flap endonuclease 1 provided in a concentration of ranging from about 0.32 U to about 4.8 U. Further provided herein are methods for nucleic acid assembly, wherein the polymerase comprises 5′ to 3′ polymerase activity. Further provided herein are methods for nucleic acid assembly, wherein the polymerase is a DNA polymerase. Further provided herein are methods for nucleic acid assembly, wherein the ligase catalyzes joining of at least two nucleic acids. Further provided herein are methods for nucleic acid assembly, wherein a concentration of the exonuclease III is in a range of about 0.1 U to about 10 U. Further provided herein are methods for nucleic acid assembly, wherein a concentration of the polymerase is in a range of about 0.01 U to about 2 U. Further provided herein are methods for nucleic acid assembly, wherein a concentration of the ligase is up to about 2.0 U.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A depicts a schematic for flap endonuclease mediated nucleic acid assembly.

FIG. 1B depicts a schematic for flap endonuclease mediated nucleic acid assembly using a bridge assembly.

FIG. 2 depicts systems for polynucleotide synthesis and flap endonuclease mediated nucleic acid assembly.

FIG. 3 illustrates a computer system.

FIG. 4 is a block diagram illustrating architecture of a computer system.

FIG. 5 is a block diagram of a multiprocessor computer system using a shared virtual address memory space.

FIG. 6 is a diagram demonstrating a network configured to incorporate a plurality of computer systems, a plurality of cell phones and personal data assistants, and Network Attached Storage (NAS).

FIG. 7 is a plot from a BioAnalyzer reading with nucleotide bases on the x-axis and fluorescent units on the y-axis.

FIG. 8 is a plot from a BioAnalyzer reading with nucleotide bases on the x-axis and fluorescent units on the y-axis.

FIG. 9 is a plot of colony forming units (CFU) with varying concentrations of ExoIII and Fen1.

FIG. 10 is a plot of colony forming units (CFU) and percentage of correct assembly of flap endonuclease mediated nucleic acid assembly reactions with varying enzyme concentrations

FIG. 11 is a plot of colony forming units (CFU) of a 1.8 kb assembly.

FIG. 12 is a plot of flap endonuclease mediated nucleic acid assembly with two fragments of DNA.

FIG. 13 is a plot of colony forming units (y-axis) for several genes (x-axis) of flap endonuclease mediated nucleic acid assembly with multiple fragments of DNA into a DNA vector.

FIG. 14A is a plot of colony forming units (y-axis) for several genes (x-axis) of flap endonuclease mediated nucleic acid assembly with increased concentration of ExoIII with multiple fragments of DNA into a DNA vector.

FIG. 14B is a plot of next generation sequence analysis of assembled genes.

FIG. 14C is a plot of samples of assembly rate.

FIG. 15A is a plot of average colony forming units (CFU) (y-axis) of flap endonuclease mediated nucleic acid assembly using a nucleic acid bridge.

FIG. 15B is a plot of colony forming units (CFU) on a log scale (y-axis) of flap endonuclease mediated nucleic acid assembly using a nucleic acid bridge.

FIG. 16A is a plot of colony forming units (CFU, y-axis) of flap endonuclease mediated nucleic acid assembly of genes (x-axis) using 1, 2, 3, 4, 5, 6, 7, 8, 9, 10, 11, or 12 fragments.

FIG. 16B is a plot of next generation sequence analysis of percent population sequenced (y-axis) and assembled genes (x-axis) using 1, 2, 3, 4, 5, 6, 7, 8, 9, 10, 11, or 12 fragments.

FIG. 16C is a plot of pass rate (y-axis) for genes (x-axis) using 1, 2, 3, 4, 5, 6, 7, 8, 9, 10, 11, or 12 fragments.

FIG. 17 is a plot of colony forming units (CFU, y-axis) of flap endonuclease mediated nucleic acid assembly using Method 2 and Method 3 for two genes using 10 minute and 30 minute incubation times.

FIG. 18A is a plot from a BioAnalyzer reading with nucleotide bases on the x-axis and fluorescent units on the y-axis.

FIG. 18B is a plot from a BioAnalyzer reading with incubation time on the x-axis and fluorescent units on the y-axis.

FIGS. 19A-19B are plots of colony forming units (y-axis) for different numbers of fragments (x-axis) using flap endonuclease mediated nucleic acid assembly, Comparator 1, and Comparator 2 methods.

FIGS. 20A-20B are plots of assembly error for flap endonuclease mediated nucleic acid assembly, Comparator 1 assembly, and Comparator 2 assembly.

FIG. 20C is a plot of percentage of total count of assembly (y-axis) comparing endonuclease mediated nucleic acid assembly, Comparator 1 assembly, and Comparator 2 assembly (x-axis).

FIG. 21A is a plot of assembled constructs pre-PCR amplification and post-amplification for various numbers of inserts.

FIG. 21B is a graph of percentage of total count of assembly (y-axis) for different numbers of fragments (x-axis) using flap endonuclease mediated nucleic acid assembly, Comparator 1, and Comparator 2 methods.

FIG. 21C is a plot of CFU percentage of population and NGS results using flap endonuclease mediated nucleic acid assembly.

FIG. 21D is a plot of percentage of distribution of correctly assembled constructs and misassembled or incorrect constructs.

FIG. 22A is a graph of observed frequency per 5,400 (y-axis) compared to the various GC categories (x-axis) of an amplified oligonucleotide population prior to assembly.

FIG. 22B is a graph of observed frequency per 5,400 (y-axis) compared to the various GC categories (x-axis) of oligonucleotide population assembled by flap endonuclease mediated nucleic acid assembly.

FIG. 23 is a graph of gene level results from multiplexed gene assembly reactions.

### DETAILED DESCRIPTION

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “nucleic acid” encompasses double- or triple-stranded nucleic acids, as well as single-stranded molecules. In double- or triple-stranded nucleic acids, the nucleic acid strands need not be coextensive (i.e., a double-stranded nucleic acid need not be double-stranded along the entire length of both strands). Nucleic acid sequences, when provided, are listed in the 5′ to 3′ direction, unless stated otherwise. Methods described herein provide for the generation of isolated nucleic acids. Methods described herein additionally provide for the generation of isolated and purified nucleic acids. A “nucleic acid” as referred to herein can comprise at least 5, 10, 20, 30, 40, 50, 60, 70, 80, 90, 100, 125, 150, 175, 200, 225, 250, 275, 300, 325, 350, 375, 400, 425, 450, 475, 500, 600, 700, 800, 900, 1000, 1100, 1200, 1300, 1400, 1500, 1600, 1700, 1800, 1900, 2000, or more bases in length. Moreover, provided herein are methods for the synthesis of any number of polypeptide-segments encoding nucleotide sequences, including sequences encoding non-ribosomal peptides (NRPs), sequences encoding non-ribosomal peptide-synthetase (NRPS) modules and synthetic variants, polypeptide segments of other modular proteins, such as antibodies, polypeptide segments from other protein families, including non-coding DNA or RNA, such as regulatory sequences e.g. promoters, transcription factors, enhancers, siRNA, shRNA, RNAi, miRNA, small nucleolar RNA derived from microRNA, or any functional or structural DNA or RNA unit of interest. The following are non-limiting examples of polynucleotides: coding or non-coding regions of a gene or gene fragment, intergenic DNA, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), small nucleolar RNA, ribozymes, complementary DNA (cDNA), which is a DNA representation of mRNA, usually obtained by reverse transcription of messenger RNA (mRNA) or by amplification; DNA molecules produced synthetically or by amplification, genomic DNA, recombinant polynucleotides, branched polynucleotides, plasmids, vectors, isolated DNA of any sequence, isolated RNA of any sequence, nucleic acid probes, and primers. cDNA encoding for a gene or gene fragment referred to herein may comprise at least one region encoding for exon sequences without an intervening intron sequence in the genomic equivalent sequence.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

Primers referred to in the exemplary workflows mentioned herein as “universal primers,” are short polynucleotides that recognize a primer binding site common to multiple DNA fragments. However, these workflows are not limited to only use of universal primers, and fragment-specific primers may be incorporated in addition or alternatively. In addition, while exemplary workflows described herein refer to assembly of gene fragments, they are not limited as such and are applicable to the assembly of longer nucleic acids in general.

### Seamless Nucleic Acid Assembly

Provided herein are methods for assembly of nucleic acids with increased efficiency and accuracy. Further provided herein are methods of assembly of nucleic acids into long genes. Polynucleotides described herein are assembled into longer nucleic acids by assembly methods comprising an endonuclease or an exonuclease, optionally in conjunction with additional enzymes.

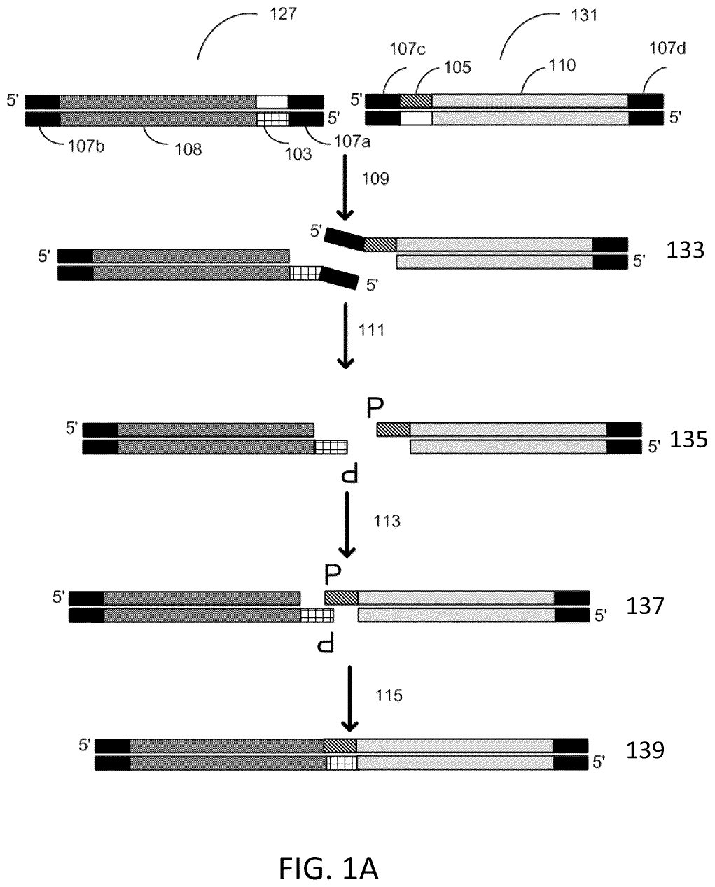

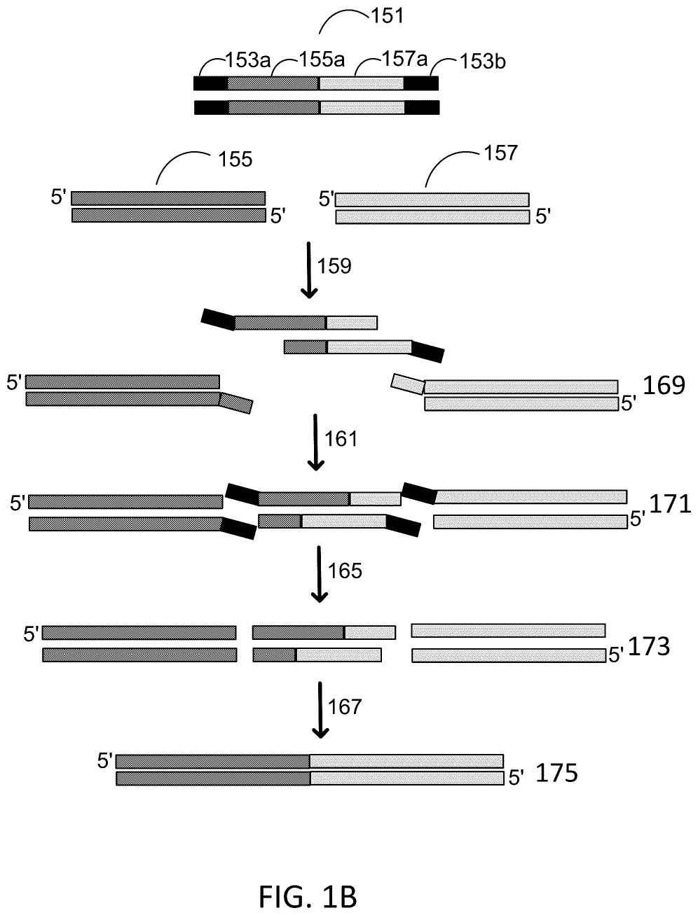

An exemplary process for assembly of nucleic acids using a flap endonuclease is depicted in FIG. 1A. Flap endonuclease mediated nucleic acid assembly is performed with first gene fragment 127 and second gene fragment 131. A bottom strand of the first gene fragment 127 is designed to comprise from 5′ to 3′ a first universal primer binding sequence 107a, a homology sequence 103, an insert sequence 108, and a second universal primer binding sequence 107b. A top strand of second gene fragment 131 is designed to comprise from 5′ to 3′ a first universal primer binding sequence 107c, a homology sequence 105, an insert sequence 110, and a second universal primer binding sequence 107d. The first gene fragment 127 and the second gene fragment 131 are contacted with a reaction mixture comprising an exonuclease, a flap endonuclease, a polymerase, and a ligase. The exonuclease digests 109 a 3′ end to expose homology sites resulting in fragments 133. In some instances, the exonuclease is exonuclease III. A flap endonuclease cleaves 111 a 5′ flap resulting in fragments 135. In some instances, the flap endonuclease is flap endonuclease 1 (FEN-1). A polymerase fills in gaps 113 and leaves a nick resulting in fragments 137. A ligase then seals 115 the nick resulting in fragments 139.

An exemplary process for assembly of nucleic acids using a flap endonuclease and a bridge assembly method is depicted in FIG. 1B. Flap endonuclease mediated nucleic acid assembly is performed with a double stranded nucleic acid bridge 151, first gene fragment 155, and second gene fragment 157. The double stranded nucleic acid bridge 151 comprises a first universal primer binding sequence 153a, a first homology sequence 155a homologous to the first gene fragment 155, a second homology sequence 157a homologous to the second gene fragment 157, and a second universal primer binding sequence 153b. The double stranded nucleic acid bridge 151, first gene fragment 155, and second gene fragment 157 are contacted with a reaction mixture comprising an exonuclease, a flap endonuclease, a polymerase, and a ligase. The exonuclease digests 159 a 3′ end to expose homology sites resulting in fragments 169. In some instances, the exonuclease is exonuclease III. A polymerase fills in gaps 161 and leaves a nick resulting in fragments 171. A flap endonuclease cleaves 165 a 5′ flap resulting in fragments 173. In some instances, the flap endonuclease is flap endonuclease 1 (FEN-1). A ligase then seals 167 the nick resulting in fragments 175. In some instances, the ligase is ampligase.

Provided herein are methods for enzymatic mediated nucleic acid assembly. In some instances, the enzymatic mediated nucleic acid assembly comprises addition of homologous sequences to gene fragments. In some instances, de novo synthesized gene fragments already comprise homology sequences. In some instances, the enzymatic mediated nucleic acid assembly comprises use of an enzymatic mixture. In some instances, the enzymatic mixture comprises an endonuclease. In some instances, the enzymatic mixture optionally comprises an exonuclease, a polymerase, or a ligase. In some instances, the enzymatic mixture comprises an exonuclease, an endonuclease, a polymerase, and a ligase. In some instances, the enzymatic mixture comprises an endonuclease, a polymerase, and a ligase. In some instances, the endonuclease is a flap endonuclease. In some instances, enzymatic mediated nucleic acid assembly results in improved efficiency. In some instances, the enzymatic mixture comprises enzymes that are not restriction enzymes. In some instances, the enzymatic mixture comprises enzymes that are structure specific enzymes. In some instances, the enzymatic mixture comprises enzymes that are structure specific enzymes and not sequence specific enzymes.

Provided herein are methods where site-specific base excision reagents comprising one or more enzymes are used as cleavage agents that cleave only a single-strand of double-stranded DNA at a cleavage site. A number of repair enzymes are suitable alone or in combination with other agents to generate such nicks. An exemplary list of repair enzymes is provided in Table 1. Homologs or non-natural variants of the repair enzymes, including those in Table 1, are also be used according to various embodiments. Any of the repair enzymes for use according to the methods and compositions described herein may be naturally occurring, recombinant or synthetic. In some instances, a DNA repair enzyme is a native or an in vitro-created chimeric protein with one or more activities. Cleavage agents, in various embodiments, comprise enzymatic activities, including enzyme mixtures, which include one or more of nicking endonucleases, AP endonucleases, glycosylases and lyases involved in base excision repair.

Repair enzymes are found in prokaryotic and eukaryotic cells. Some enzymes having applicability herein have glycosylase and AP endonuclease activity in one molecule. AP endonucleases are classified according to their sites of incision. Class I AP endonucleases and class II AP endonucleases incise DNA at the phosphate groups 3′ and 5′ to the baseless site leaving 3′-OH and 5′-phosphate termini. Class III and class IV AP endonucleases also cleave DNA at the phosphate groups 3′ and 5′ to the baseless site, but they generate a 3′-phosphate and a 5′-OH. Examples of polynucleotide cleavage enzymes used include DNA repair enzymes are listed in Table 1.

TABLE 1
DNA Repair Enzymes.

| | | Accession |
|---|---|---|
| Gene Name | Activity | Number |
| UNG | Uracil-DNA glycosylase | NM_080911 |
| SMUG1 | Uracil-DNA glycosylase | NM_014311 |
| MBD4 | Removes U or T opposite G at CpG | NM_003925 |
| | sequences | |
| TDG | Removes U, T or ethenoC opposite | NM_003211 |
| | G | |
| OGG1 | Removes 8-oxoG opposite C | NM_016821 |
| MUTYH (MYH) | Removes A opposite 8-oxoG | NM_012222 |
| NTHL1 (NTH1) | Removes Ring-saturated or | NM_002528 |
| | fragmented pyrimidines | |
| MPG | Removes 3-meA, ethenoA, | NM_002434 |
| | hypoxanthine | |
| NEIL1 | Removes thymine glycol | NM_024608 |
| NEIL2 | Removes oxidative products of | NM_145043 |
| | pyrimidines | |
| XPC | Binds damaged DNA as complex | NM_004628 |
| | with RAD23B, CETN2 | |
| RAD23B (HR23B) | Binds damaged DNA as complex | NM_002874 |
| | with XPC, CETN2 | |
| CETN2 | Binds damaged DNA as complex | NM_004344 |
| | with XPC, RAD23B | |
| RAD23A (HR23A) | Substitutes for HR23B | NM_005053 |
| XPA | Binds damaged DNA in preincision | NM_000380 |
| | complex | |
| RPA1 | Binds DNA in preincision complex | NM_002945 |
| RPA2 | Binds DNA in preincision complex | NM_002946 |
| RPA3 | Binds DNA in preincision complex | NM_002947 |
| ERCC5 (XPG) | 3′ incision | NM_000123 |
| ERCC1 | 5′ incision subunit | NM_001983 |
| ERCC4 (XPF) | 5′ incision subunit | NM_005236 |
| LIG1 | DNA joining | NM_000234 |
| CKN1(CSA) | Cockayne syndrome; Needed for | NM_000082 |
| | transcription-coupled NER | |
| ERCC6 (CSB) | Cockayne syndrome; Needed for | NM_000124 |
| | transcription-coupled NER | |
| XAB2 (HCNP) | Cockayne syndrome; Needed for | NM_020196 |
| | transcription-coupled NER | |
| DDB1 | Complex defective in XP group E | NM_001923 |
| DDB2 | DDB1, DDB2 | NM_000107 |
| MMS19L (MMS19) | Transcription and NER | NM_022362 |
| FEN1 (DNase IV) | Flap endonuclease | NM_004111 |
| SPO11 | endonuclease | NM_012444 |
| FLJ35220 | incision 3′ of hypoxanthine and | NM_173627 |
| (ENDOV) | uracil | |
| FANCA | Involved in tolerance or repair of | NM_000135 |
| | DNA crosslinks | |
| FANCB | Involved in tolerance or repair of | NM_152633 |
| | DNA crosslinks | |
| FANCC | Involved in tolerance or repair of | NM_000136 |
| | DNA crosslinks | |
| FANCD2 | Involved in tolerance or repair of | NM_033084 |
| | DNA crosslinks | |
| FANCE | Involved in tolerance or repair of | NM_021922 |
| | DNA crosslinks | |
| FANCF | Involved in tolerance or repair of | NM_022725 |
| | DNA crosslinks | |
| FANCG (XRCC9) | Involved in tolerance or repair of | NM_004629 |
| | DNA crosslinks | |
| FANCL | Involved in tolerance or repair of | NM_018062 |
| | DNA crosslinks | |
| DCLRE1A (SNM1) | DNA crosslink repair | NM_014881 |
| DCLRE1B | Related to SNM1 | NM_022836 |
| (SNM1B) | | |
| NEIL3 | Resembles NEIL1 and NEIL2 | NM_018248 |
| ATRIP (TREX1) | ATR-interacting protein 5′ | NM_130384 |
| | alternative ORF of the | |
| | TREX1/ATRIP gene | |
| NTH | Removes damaged pyrimidines | NP_416150.1 |
| NEI | Removes damaged pyrimidines | NP_415242.1 |
| NFI | Deoxyinosine 3′ endonuclease | NP_418426.1 |
| MUTM | Formamidopyrimidine DNA | NP_418092.1 |
| | glycosylase | |
| UNG | Uracil-DNA glycosylase | NP_417075.1 |
| UVRA | DNA excision repair enzyme | NP_418482.1 |
| | complex | |
| UVRB | DNA excision repair enzyme | NP_415300.1 |
| | complex | |
| UVRC | DNA excision repair enzyme | NP_416423.3 |
| | complex | |
| DENV | Pyrimidine dimer glycosylase | NP_049733.1 |

Provided herein are methods for enzymatic mediated nucleic acid assembly, wherein gene fragments or genes for assembly comprise a homology sequence. In some instances, the homology sequence comprises at least or about 5, 10, 20, 30, 40, 50, 60, 70, 80, 90, 100, or more than 100 base pairs. In some instance, the number of base pairs is 40 base pairs. In some instances, the number of base pairs has a range of about 5 to 100, 10 to 90, 20 to 80, 30 to 70, or 40 to 60 base pairs.

Provided herein are methods for enzymatic mediated nucleic acid assembly, wherein gene fragments or genes for assembly do not comprise a homology sequence. In some instances, methods for enzymatic mediated nucleic acid assembly of de novo synthesized gene fragments without a homology sequence comprise assembly using a nucleic acid bridge. In some instances, the nucleic acid bridge comprises DNA or RNA. In some instances, the nucleic acid bridge comprises DNA. In some instances, the nucleic acid bridge is double stranded. In some instances, the nucleic acid bridge is single stranded.

Provided herein are methods for enzymatic mediated nucleic acid assembly using a nucleic acid bridge, wherein the nucleic acid bridge comprises one or more universal primer binding sequences. In some instances, the nucleic acid bridge comprises at least or about 1, 2, 3, 4, 5, 6, 7, 8, or more than 8 universal primer binding sequences. In some instances, the nucleic acid bridge further comprises a homology sequence. In some instances, the homology sequence is homologous to a de novo synthesized gene fragment. In some instances, the nucleic acid bridge further comprises one or more homology sequences. For example, the nucleic acid bridge comprises one or more homology sequences that are homologous to different de novo synthesized gene fragments. In some instances, the nucleic acid bridge comprises 1, 2, 3, 4, 5, 6, 7, 8, 9, 10, or more than 10 homology sequences. In some instances, the homology sequence comprises at least or about 5, 10, 20, 30, 40, 50, 60, 70, 80, 90, 100, or more than 100 base pairs. In some instances, the number of base pairs is 40 base pairs. In some instance, the number of base pairs is 50 base pairs. In some instances, the number of base pairs has a range of about 5 to 100, 10 to 90, 20 to 80, 30 to 70, or 40 to 60 base pairs.

Provided herein are methods for enzymatic mediated nucleic acid assembly, wherein a double stranded nucleic acid is contacted with an enzyme comprising exonuclease activity. In some instances, the exonuclease comprises 3′ exonuclease activity. Exemplary exonucleases comprising 3′ exonuclease activity include, but are not limited to, exonuclease I, exonuclease III, exonuclease V, exonuclease VII, and exonuclease T. In some instances, the exonuclease comprises 5′ exonuclease activity. Exemplary exonucleases comprising 5′ exonuclease activity include, but are not limited to, exonuclease II, exonuclease IV, exonuclease V, exonuclease VI, exonuclease VII, exonuclease VIII, T5 exonuclease, and T7 exonuclease. In some instances, the exonuclease is exonuclease III (ExoIII). Exonucleases include wild-type exonucleases and derivatives, chimeras, and/or mutants thereof. Mutant exonucleases include enzymes comprising one or more mutations, insertions, deletions or any combination thereof within the amino acid or nucleic acid sequence of an exonuclease.

In some instances, the exonuclease is used at a temperature optimal for enzymatic activity, for example, a temperature in a range of about 25-80° C., 25-70° C., 25-60° C., 25-50° C., or 25-40° C. In some instances, the temperature is about 37° C. In some instances, the temperature is about 50° C. In some instances, the temperature is about 55° C. In some instances, the temperature is about 65° C. In some instances, the temperature is at least or about 15° C., 20° C., 25° C., 30° C., 35° C., 40° C., 45° C., 50° C., 55° C., 60° C., 65° C., 70° C., 75° C., 80° C., or more than 80° C.

In some instances, methods for enzymatic mediated nucleic acid assembly do not comprise using an exonuclease. In some instances, methods for enzymatic mediated nucleic acid assembly comprise using an exonuclease. In some instances, one or more exonucleases are used. For example, at least or about 1, 2, 3, 4, 5, 6, or more than 6 exonucleases are used. In some instances, the exonuclease comprises 5′ to 3′ exonuclease activity. In some instances, the exonuclease comprises 3′ to 5′ exonuclease activity. In some instances, methods comprise contacting double stranded DNA with an endonuclease. In some instances, the endonuclease is a flap endonuclease. In some instances, methods comprise contacting double stranded DNA with a flap endonuclease, a ligase, or a polymerase. In some instances, the flap endonuclease is flap endonuclease 1.

Provided herein are methods wherein a double stranded nucleic acid is treated with an enzyme comprising endonuclease activity. In some instances, the endonuclease comprises 5′ nuclease activity. In some instances, the endonuclease comprises 3′ nuclease activity. In some instances, the endonuclease is a flap endonuclease. In some instances, the flap endonuclease comprises 5′ nuclease activity. In some instances, the flap endonuclease is a member of a 5′-nuclease family of enzymes. Exemplary 5′-nuclease enzymes include, but are not limited to, flap endonuclease 1, exonuclease 1, xeroderma pigmentosum complementation group G (XPG), Dna2, and gap endonuclease 1 (GEN1). In some instances, the flap endonuclease is flap endonuclease 1. In some instances, the flap endonuclease comprises 3′ nuclease activity. Exemplary flap endonucleases with 3′ nuclease activity include, but are not limited to, RAG1, RAG2, and MUS81. In some instances, the flap endonuclease is an archaeal, bacteria, yeast, plant, or mammalian flap endonuclease. Exemplary 5′-nuclease and 3′ nuclease enzymes are seen in Table 2.

TABLE 2
Exemplary Nuclease Enzymes

| Name | Species | Protein Accession Number |
|---|---|---|
| flap endonuclease 1 | Homo sapiens | NP_004102.1 |
| flap endonuclease 1 | Mus musculus | NP_001258544.1 |
| flap endonuclease 1 | Pyrococcus furiosis | O93634 |
| exonuclease 1 | Homo sapiens | AAH07491.1 |
| XPG | Homo sapiens | EAX09071.1 |
| Dna2 | Homo sapiens | NP_001073918.2 |
| GEN1 | Homo sapiens | NP_001123481.2 |
| RAG1 | Homo sapiens | AAH37344.1 GI |
| RAG2 | Homo sapiens | NP_001230715. |
| MUS81 | Homo sapiens | Q96NY9.3 |

In some instances, the endonuclease is used at a temperature optimal for enzymatic activity, for example, a temperature of 25-80° C., 25-70° C., 25-60° C., 25-50° C., or 25-40° C. In some instances, the temperature is about 50° C. In some instances, the temperature is about 55° C. In some instances, the temperature is about 65° C. In some instances, the temperature is at least or about 15° C., 20° C., 25° C., 30° C., 35° C., 40° C., 45° C., 50° C., 55° C., 60° C., 65° C., 70° C., 75° C., 80° C., or more than 80° C. In some instances, the endonuclease is a thermostable endonuclease. A thermostable endonuclease may include endonucleases that are functional at temperatures at least or about 60° C., 65° C., 70° C., 75° C., 80° C., or more than 80° C. In some instances, the endonuclease is a flap endonuclease. In some instances, the flap endonuclease is a thermostable flap endonuclease.

Provided herein are methods for nucleic acid assembly, wherein the ratio of the endonuclease to the exonuclease is from about 0.1:1 to about 1:5. In some instances, the endonuclease is a flap endonuclease. In some instances, the ratio of the endonuclease to the exonuclease is at least or about 0.2:1, 0.25:1, 0.5:1, 0.75:1, 1:1, 1:1.5, 1:2, 1:3, 1:4, 1:5, or more than 1:5. In some instances, the ratio of the endonuclease to the exonuclease is at least or about 1:1, 1:0.9, 1:0.85, 1:0.8, 1:0.75, 1:0.7, 1:0.65, 1:0.6, 1:0.55, 1:0.5, 1:0.45, 1:0.4, 1:0.35, 1:0.3, 1:0.25, 1:0.2, 1:0.15, 1:0.1, or less than 1:0.1.

Provided herein are methods for nucleic acid assembly comprising an exonuclease, wherein the concentration of the exonuclease is from about 0.1 U to about 20 U or more. For example, the concentration of the exonuclease is at least or about 0.1 U, 0.25 U, 0.5 U, 0.75 U, 1 U, 1.6 U, 2 U, 3 U, 4 U, 5 U, 6 U, 7 U, 8 U, 9 U, 10 U, 12 U, 14 U, 16 U, 18 U, 20 U, or more than 20 U. In some instances, the concentration of the exonuclease is in a range of about 0.5 U to about 1.0 U. In some instances, the concentration of the exonuclease is from about 1.0 U to about 2.0 U. In some instances, the concentration of the exonuclease is about 1.6 U. In some instances, the concentration of the exonuclease is about 5.0 U. In some instances, the concentration of the exonuclease ranges from about 0.1 U to 20 U, 0.25 U to 18 U, 0.5 U to 16 U, 0.75 U to 14 U, 1 U to 12 U, 2 U to 10 U, 3 U to 9 U, or 4 U to 8 U.

Methods described herein for enzymatic mediated nucleic acid assembly may comprise an endonuclease, wherein the concentration of the endonuclease is from about 0.25 U to about 12 U or more. In some instances, the endonuclease is a flap endonuclease. Exemplary concentrations of the endonuclease, include, but are not limited to, at least or about 0.25 U, 0.5 U, 0.75 U, 1 U, 2 U, 3 U, 4 U, 5 U, 6 U, 7 U, 8 U, 9 U, 10 U, 11 U, 12 U, or more than 12 U. In some instances, the concentration of the endonuclease is 0.32 U. In some instances, the concentration of the endonuclease is 1.6 U. In some instances, the concentration of the endonuclease is in a range of about 0.32 U to about 4.8 U. In some instances, the concentration of the endonuclease is in a range of about 0.25 U to 12 U, 0.5 U to 11 U, 0.75 U to 10 U, 1 U to 9 U, 2 U to 8 U, 3 U to 7 U, or 4 U to 6 U.

Provided herein are methods for enzymatic mediated nucleic acid assembly, wherein a double stranded nucleic acid is mixed with a polymerase. In some instances, the polymerase is a DNA polymerase. In some instances, the polymerase is a high fidelity polymerase. A high fidelity polymerase may include polymerases that result in accurate replication or amplification of a template nucleic acid. In some instances, the DNA polymerase is a thermostable DNA polymerase. The DNA polymerase may be from any family of DNA polymerases including, but not limited to, Family A polymerase, Family B polymerase, Family C polymerase, Family D polymerase, Family X polymerase, and Family Y polymerase. In some instances, the DNA polymerase is from a genus including, but not limited to, Thermus, Bacillus, Thermococcus, Pyrococcus, Aeropyrum, Aquifex, Sulfolobus, Pyrolobus, or Methanopyrus.

Polymerases described herein for use in an amplification reaction may comprise various enzymatic activities. Polymerases are used in the methods of the invention, for example, to extend primers to produce extension products. In some instances, the DNA polymerase comprises 5′ to 3′ polymerase activity. In some instances, the DNA polymerase comprises 3′ to 5′ exonuclease activity. In some instances, the DNA polymerase comprises proofreading activity. Exemplary polymerases include, but are not limited to, DNA polymerase (I, II, or III), T4 DNA polymerase, T7 DNA polymerase, Bst DNA polymerase, Bca polymerase, Vent DNA polymerase, Pfu DNA polymerase, and Taq DNA polymerase. Non-limiting examples of thermostable DNA polymerases include, but are not limited to, Taq, Phusion® DNA polymerase, Q5® High Fidelity DNA Polymerase, LongAmp® DNA polymerase, Expand High Fidelity polymerase, HotTub polymerase, Pwo polymerase, Tfl polymerase, Tli polymerase, UlTma polymerase, Pfu polymerase, KOD DNA polymerase, JDF-3 DNA polymerase, PGB-D DNA polymerase, Tgo DNA polymerase, Pyrolobus furmarius DNA polymerase, Vent polymerase, and Deep Vent polymerase.

Described herein are methods comprising a DNA polymerase, wherein a concentration of the DNA polymerase is from about 0.1 U to about 2 U, or more than 2 U. In some instances, the concentration of the DNA polymerase is about 0.1 U. In some instances, the concentration of the DNA polymerase is about 0.2 U. In some instances, the concentration of the DNA polymerase is about 0.01 U. In some instances, the concentration of the DNA polymerase is in a range of at least or about 0.005 U to 2 U, 0.005 U to 1 U, 0.005 U to.5 U, 0.01 U to 1 U, 0.1 U to 0.5 U, 0.1 U to 0.5 U, 0.1 U to 1 U, 0.1 U to 1.5 U, 0.1 U to 2 U, 0.5 U to 1.0 U, 0.5 U to 1.5 U, 0.5 U to 2 U, 1 U to 1.5 U, 1.0 U to 2.0 U, or 1.5 U to 2 U.

The DNA polymerase for use in methods described herein are used at a temperature optimal for enzymatic activity, for example, a temperature of 25-80° C., 25-70° C., 25-60° C., 25-50° C., or 25-40° C. In some instances, the temperature is about 50° C. In some instances, the temperature is about 55° C. In some instances, the temperature is about 65° C. In some instances, the temperature is at least or about 15° C., 20° C., 25° C., 30° C., 35° C., 40° C., 45° C., 50° C., 55° C., 60° C., 65° C., 70° C., 75° C., 80° C., or more than 80° C.

Methods described herein for enzymatic mediated nucleic acid assembly may comprise an amplification reaction, wherein the amplification reaction comprises a universal primer binding sequence. In some instances, the universal primer binding sequence is capable of binding the same 5′ or 3′ primer. In some instances, the universal primer binding sequence is shared among a plurality of target nucleic acids in the amplification reaction.

Provided herein are methods for enzymatic mediated nucleic acid assembly, wherein a double stranded nucleic acid is treated with a ligase. Ligases as described herein may function to join nucleic acid fragments. For example, the ligase functions to join adjacent 3′-hydroxylated and 5′-phosphorylated termini of DNA. Ligases include, but are not limited to, E. coli ligase, T4 ligase, mammalian ligases (e.g., DNA ligase I, DNA ligase II, DNA ligase III, DNA ligase IV), thermostable ligases, and fast ligases. In some instances, the ligase is a thermostable ligase. In some instances, the ligase is Ampligase.

The concentration of the ligase may vary. In some instances, the concentration of the ligase is in a range of about 0 U to about 2 U. An exemplary concentration of the ligase is about 0.5 U. In some instances, the concentration of the ligase is about 1.0 U. In some instances, the concentration of the ligase is about 5.0 U. In some instances, the concentration of the ligase is in a range of at least or about 0 U to 0.25 U, 0 U to 0.5 U, 0 U to 1 U, 0 U to 1.5 U, 0 U to 2 U, 0.25 U to 0.5 U, 0.25 U to 1.0 U, 0.25 U to 1.5 U, 0.25 U to 2.0 U, 0.5 U to 1.0 U, 0.5 U to 1.5 U, 0.5 U to 2.0 U, 1.0 U to 1.5 U, 1.0 U to 2.0 U, 1.5 U to 2.0 U, 2.0 U to 4.0 U, 4.0 U to 6.0 U, 4.0 U to 8.0 U, 6.0 U to 10.0 U.

In some instances, the ligase is used at a temperature optimal for enzymatic activity, for example, a temperature of 25-80° C., 25-70° C., 25-60° C., 25-50° C., or 25-40° C. In some instances, the temperature is about 50° C. In some instances, the temperature is about 55° C. In some instances, the temperature is about 65° C. In some instances, the temperature is at least or about 15° C., 20° C., 25° C., 30° C., 35° C., 40° C., 45° C., 50° C., 55° C., 60° C., 65° C., 70° C., 75° C., 80° C., or more than 80° C.

Provided herein are methods for enzymatic mediated nucleic acid assembly, wherein a number of gene fragments are assembled. In some instances, the gene fragments are assembled processively or sequentially. In some instances, the gene fragments are assembled into a vector. In some instances, the gene fragments are assembled for long linear gene assembly. In some instances, the number of gene fragments is at least or about 2, 3, 4, 5, 6, 7, 8, 9, 10, or more than 10 gene fragments. In some instances, the number of gene fragments is at least or about 10, 11, 12, 13, 14, 15, 16, 17, 18, 19, 20, or more than 20 gene fragments. In some instances, the number of gene fragments is in a range of about 1 to 2, 1 to 3, 1 to 4, 1 to 5, 1 to 6, 1 to 7, 1 to 8, 1 to 9, 1 to 10, 2 to 3, 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 10, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10, or 9 to 10. In some instances, the number of gene fragments is about 1 to about 20, about 2 to about 18, about 3 to about 17, about 4 to about 16, about 6 to about 14, or about 8 to about 12.

Provided herein are methods for enzymatic mediated nucleic acid assembly, wherein a ratio of gene fragments assembled is about 0.2:1, 0.25:1, 0.5:1, 0.75:1, 1:1, 1:1.5, 1:2, 1:3, 1:4, 1:5, or more than 1:5. For example, if two gene fragments are assembled, a ratio of the first gene fragment to the second gene fragment is 1:1. In some instances, a ratio of the first gene fragment to the second gene fragment is at least or about 1:1, 1:0.9, 1:0.85, 1:0.8, 1:0.75, 1:0.7, 1:0.65, 1:0.6, 1:0.55, 1:0.5, 1:0.45, 1:0.4, 1:0.35, 1:0.3, 1:0.25, 1:0.2, 1:0.15, 1:0.1, or less than 1:0.1.

Methods as described herein for enzymatic mediated nucleic acid assembly may comprise assembly of one or more gene fragments into a vector, wherein a ratio of the one or more gene fragments to the vector varies. In some instances, a ratio of the one or more gene fragments to the vector is at least or about 0.2:1, 0.25:1, 0.5:1, 0.75:1, 1:1, 1:1.5, 1:2, 1:3, 1:4, 1:5, or more than 1:5. In some instances, a ratio of the one or more gene fragments to the vector is at least or about 1:1, 1:0.9, 1:0.85, 1:0.8, 1:0.75, 1:0.7, 1:0.65, 1:0.6, 1:0.55, 1:0.5, 1:0.45, 1:0.4, 1:0.35, 1:0.3, 1:0.25, 1:0.2, 1:0.15, 1:0.1, or less than 1:0.1.

Methods as described herein for enzymatic mediated nucleic acid assembly may comprise assembly of oligonucleotide populations for assembly into a vector. In some instances, overlap extension PCR is performed for assembly of oligonucleotide populations. In some instances, the oligonucleotide population comprises at least or about 2, 3, 4, 5, 6, 7, 8, 9, 10, 15, 20, 25, 30, 35, 40, 45, 50, 60, 70, 80, 90, 100, 200, or more than 200 oligonucleotides. In some instances, the oligonucleotide population are assembled to generate a long nucleic acid comprising at least or about 50, 100, 200, 250 300, 400, 500, 600, 700, 800, 900, 1000, 1100, 1300, 1400, 1500, 1600, 1700, 1800, 2000, 2400, 2600, 2800, 3000, 3200, 3400, 3600, 3800, 4000, 4200,4400, 4600, 4800, 5000, 6000, 7000, 8000, 9000, 10000, or more than 10000 bases.

Methods as described herein for enzymatic mediated nucleic acid assembly may comprise multiplexed gene assembly. In some instances, multiple sequences are assembled in a single reaction. In some instances, at least or about 2, 3, 4, 5, 6, 7, 8, 9, 10, 15, 20, 25, 30, 35, 40, 45, 50, 60, 70, 80, 90, 100, 200, or more than 200 sequences are assembled in a single reaction. Sequences assembled by multiplex gene assembly, in some instances, are inserted into a vector.

Methods for enzymatic mediated nucleic acid assembly may comprise assembly of one or more gene fragments using a nucleic acid bridge, wherein a ratio of the one or more gene fragments to the nucleic acid bridge varies. In some instances, a ratio of the one or more gene fragments to the nucleic acid bridge is at least or about 0.2:1, 0.25:1, 0.5:1, 0.75:1, 1:1, 1:1.5, 1:2, 1:3, 1:4, 1:5, or more than 1:5. In some instances, a ratio of the one or more gene fragments to the nucleic acid bridge is at least or about 1:1, 1:0.9, 1:0.85, 1:0.8, 1:0.75, 1:0.7, 1:0.65, 1:0.6, 1:0.55, 1:0.5, 1:0.45, 1:0.4, 1:0.35, 1:0.3, 1:0.25, 1:0.2, 1:0.15, 1:0.1, or less than 1:0.1.

Provided herein are methods for enzymatic mediated nucleic acid assembly of gene fragments, wherein a total size of the number of gene fragments that are assembled is at least or about 50, 100, 200, 250 300, 400, 500, 600, 700, 800, 900, 1000, 1100, 1300, 1400, 1500, 1600, 1700, 1800, 2000, 2400, 2600, 2800, 3000, 3200, 3400, 3600, 3800, 4000, 4200,4400, 4600, 4800, 5000, 6000, 7000, 8000, 9000, 10000, or more than 10000 bases. In some instances, a total size of the number of gene fragments that are assembled is in a range of about 300 to 1,000, 300 to 2,000, 300 to 3,000, 300 to 4,000, 300 to 5,000, 300 to 6,000, 300 to 7,000, 300 to 8,000, 300 to 9,000, 300 to 10,000, 1,000 to 2,000, 1,000 to 3,000, 1,000 to 4,000, 1,000 to 5,000, 1,000 to 6,000, 1,000 to 7,000, 1,000 to 8,000, 1,000 to 9,000, 1,000 to 10,000, 2,000 to 3,000, 2,000 to 4,000, 2,000 to 5,000, 2,000 to 6,000, 2,000 to 7,000, 2,000 to 8,000, 2,000 to 9,000, 2,000 to 10,000, 3,000 to 4,000, 3,000 to 5,000, 3,000 to 6,000, 3,000 to 7,000, 3,000 to 8,000, 3,000 to 9,000, 3,000 to 10,000, 4,000 to 5,000, 4,000 to 6,000, 4,000 to 7,000, 4,000 to 8,000, 4,000 to 9,000, 4,000 to 10,000, 5,000 to 6,000, 5,000 to 7,000, 5,000 to 8,000, 5,000 to 9,000, 5,000 to 10,000, 6,000 to 7,000, 6,000 to 8,000, 6,000 to 9,000, 6,000 to 10,000, 7,000 to 8,000, 7,000 to 9,000, 7,000 to 10,000, 8,000 to 9,000, 8,000 to 10,000, or 9,000 to 10,000 bases.

Methods described herein comprising enzymatic mediated nucleic acid assembly result in a high percentage of correct assembly. In some instances, the percentage of correct assembly is at least or about 60%, 65%, 70%, 75%, 80%, 85%, 90%, 95%, 97%, 99%, or more than 99%. In some instances, the percentage of average correct assembly is at least or about 60%, 65%, 70%, 75%, 80%, 85%, 90%, 95%, 97%, 99%, or more than 99%. In some instances, the percentage of correct assembly is 100%.

Methods as described herein comprising enzymatic mediated nucleic acid assembly result in a low percentage of misassembly. In some instances, the percentage misassembly rate is at most 5%, 10%, 15%, 20%, 25%, 30%, 35%, 40%, 45%, 50%, 55%, or 60%. In some instances, the percentage misassembly rate is about 1% to about 25%, about 5% to about 20%, or about 10% to about 15%. In some instances, the average misassembly rate is at most 5%, 10%, 15%, 20%, 25%, 30%, 35%, 40%, 45%, 50%, 55%, or 60%. In some instances, the average misassembly rate is about 1% to about 25%, about 5% to about 20%, or about 10% to about 15%.

Methods described herein comprising enzymatic mediated nucleic acid assembly result in increased efficiency. In some instances, efficiency is measured by number of colony forming units. In some instances, methods described herein result in at least or about 100, 200, 300, 400, 500, 600, 700, 800, 900, 1000, 2000, 3000, 4000, 5000, 6000, 7000, 8000, 9000, 10000, 12000, 14000, 16000, 18000, 20000, 25000, 30000, 35000, 40000, 50000, 60000, 70000, 80000, 90000, 100000, or more than 100000 colony forming units.

### Systems for Synthesis of Nucleic Acids and Seamless Assembly

### Polynucleotide Synthesis

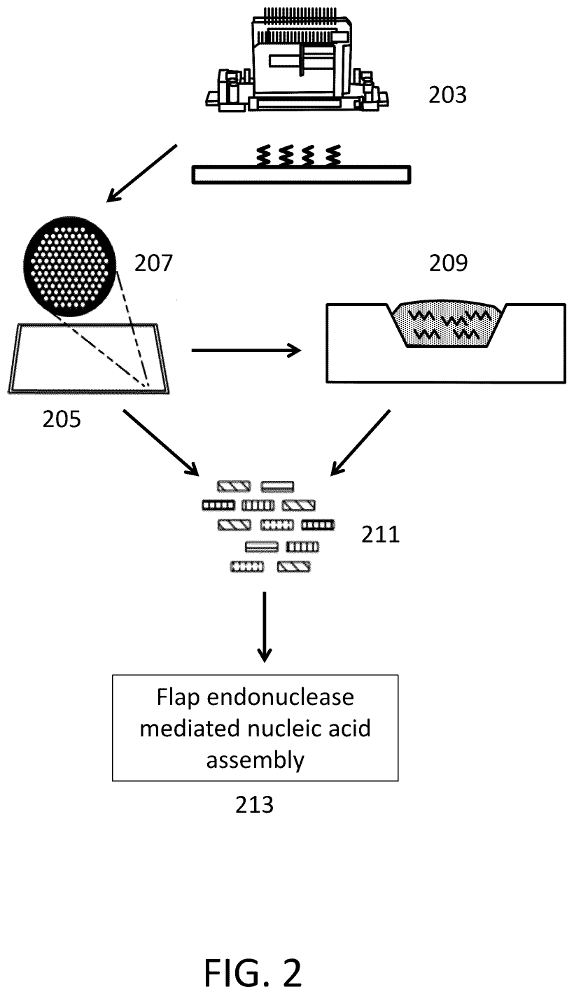

Provided herein are methods for seamless assembly of nucleic acids following generation of polynucleotides by de novo synthesis by methods described herein. An exemplary workflow is seen in FIG. 2. A computer readable input file comprising a nucleic acid sequence is received. A computer processes the nucleic acid sequence to generate instructions for synthesis of the polynucleotide sequence or a plurality of polynucleotide sequences collectively encoding the nucleic acid sequence. Instructions are transmitted to a material deposition device 203 for synthesis of the plurality of polynucleotides based on the plurality of nucleic acid sequences. The material deposition device 203, such as a polynucleotide acid synthesizer, is designed to release reagents in a step wise fashion such that multiple polynucleotides extend, in parallel, one residue at a time to generate oligomers with a predetermined nucleic acid sequence. The material deposition device 203 generates oligomers on an array 205 that includes multiple clusters 207 of loci for polynucleotide acid synthesis and extension. However, the array need not have loci organized in clusters. For example, the loci can be uniformly spread across the array. De novo polynucleotides are synthesized and removed from the plate and an assembly reaction commenced in a collection chamber 209 followed by formation population of longer polynucleotides 211. The collection chamber may comprise a sandwich of multiple surfaces (e.g., a top and bottom surface) or well or channel in containing transferred material from the synthesis surface. De novo polynucleotides can also be synthesized and removed from the plate to form a population of longer polynucleotides 211. The population of longer polynucleotides 211 can then be partitioned into droplets or subject to PCR. The population of longer polynucleotides 211 is then subject to nucleic acid assembly by flap endonuclease mediated nucleic acid assembly 213.

Provided herein are systems for seamless assembly of nucleic acids following generation of polynucleotides by de novo synthesis by methods described herein. In some instances, the system comprises a computer, a material deposition device, a surface, and a nucleic acid assembly surface. In some instances, the computer comprises a readable input file with a nucleic acid sequence. In some instances, the computer processes the nucleic acid sequence to generate instructions for synthesis of the polynucleotide sequence or a plurality of polynucleotide sequences collectively encoding for the nucleic acid sequence. In some instances, the computer provides instructions to the material deposition device for the synthesis of the plurality of polynucleotide acid sequences. In some instances, the material deposition device deposits nucleosides on the surface for an extension reaction. In some instances, the surface comprises a locus for the extension reaction. In some instances, the locus is a spot, well, microwell, channel, or post. In some instances, the plurality of polynucleotide acid sequences is synthesized following the extension reaction. In some instances, the plurality of polynucleotide acid sequences is removed from the surface and prepared for nucleic acid assembly. In some instances, the nucleic acid assembly comprises flap endonuclease mediated nucleic acid assembly.

Provided herein are methods for polynucleotide synthesis involving phosphoramidite chemistry. In some instances, polynucleotide synthesis comprises coupling a base with phosphoramidite. In some instances, polynucleotide synthesis comprises coupling a base by deposition of phosphoramidite under coupling conditions, wherein the same base is optionally deposited with phosphoramidite more than once, i.e., double coupling. In some instances, polynucleotide synthesis comprises capping of unreacted sites. In some cases, capping is optional. In some instances, polynucleotide synthesis comprises oxidation. In some instances, polynucleotide synthesis comprises deblocking or detritylation. In some instances, polynucleotide synthesis comprises sulfurization. In some cases, polynucleotide synthesis comprises either oxidation or sulfurization. In some instances, between one or each step during a polynucleotide synthesis reaction, the substrate is washed, for example, using tetrazole or acetonitrile. Time frames for any one step in a phosphoramidite synthesis method include less than about 2 min, 1 min, 50 sec, 40 sec, 30 sec, 20 sec, or 10 sec.

Polynucleotide synthesis using a phosphoramidite method comprises the subsequent addition of a phosphoramidite building block (e.g., nucleoside phosphoramidite) to a growing polynucleotide chain for the formation of a phosphite triester linkage. Phosphoramidite polynucleotide synthesis proceeds in the 3′ to 5′ direction. Phosphoramidite polynucleotide synthesis allows for the controlled addition of one nucleotide to a growing nucleic acid chain per synthesis cycle. In some instances, each synthesis cycle comprises a coupling step.

Phosphoramidite coupling involves the formation of a phosphite triester linkage between an activated nucleoside phosphoramidite and a nucleoside bound to the substrate, for example, via a linker. In some instances, the nucleoside phosphoramidite is provided to the substrate activated. In some instances, the nucleoside phosphoramidite is provided to the substrate with an activator. In some instances, nucleoside phosphoramidites are provided to the substrate in a 1.5, 2, 3, 4, 5, 6, 7, 8, 9, 10, 11, 12, 13, 14, 15, 16, 17, 18, 19, 20, 25, 30, 35, 40, 50, 60, 70, 80, 90, 100-fold excess or more over the substrate-bound nucleosides. In some instances, the addition of nucleoside phosphoramidite is performed in an anhydrous environment, for example, in anhydrous acetonitrile. Following addition of a nucleoside phosphoramidite, the substrate is optionally washed. In some instances, the coupling step is repeated one or more additional times, optionally with a wash step between nucleoside phosphoramidite additions to the substrate. In some instances, a polynucleotide synthesis method used herein comprises 1, 2, 3 or more sequential coupling steps. Prior to coupling, in many cases, the nucleoside bound to the substrate is de-protected by removal of a protecting group, where the protecting group functions to prevent polymerization. A common protecting group is 4,4′-dimethoxytrityl (DMT).

Following coupling, phosphoramidite polynucleotide synthesis methods optionally comprise a capping step. In a capping step, the growing polynucleotide is treated with a capping agent. A capping step is useful to block unreacted substrate-bound 5′-OH groups after coupling from further chain elongation, preventing the formation of polynucleotides with internal base deletions. Further, phosphoramidites activated with 1H-tetrazole may react, to a small extent, with the O6 position of guanosine. Without being bound by theory, upon oxidation with I₂/water, this side product, possibly via O6-N7 migration, may undergo depurination. The apurinic sites may end up being cleaved in the course of the final deprotection of the polynucleotide thus reducing the yield of the full-length product. The O6 modifications may be removed by treatment with the capping reagent prior to oxidation with I₂/water. In some instances, inclusion of a capping step during polynucleotide synthesis decreases the error rate as compared to synthesis without capping. As an example, the capping step comprises treating the substrate-bound polynucleotide with a mixture of acetic anhydride and 1-methylimidazole. Following a capping step, the substrate is optionally washed.

In some instances, following addition of a nucleoside phosphoramidite, and optionally after capping and one or more wash steps, the substrate bound growing nucleic acid is oxidized. The oxidation step comprises oxidation of the phosphite triester into a tetracoordinated phosphate triester, a protected precursor of the naturally occurring phosphate diester internucleoside linkage. In some cases, oxidation of the growing polynucleotide is achieved by treatment with iodine and water, optionally in the presence of a weak base (e.g., pyridine, lutidine, collidine). Oxidation may be carried out under anhydrous conditions using, e.g. tert-Butyl hydroperoxide or (1S)-(+)-(10-camphorsulfonyl)-oxaziridine (CSO). In some methods, a capping step is performed following oxidation. A second capping step allows for substrate drying, as residual water from oxidation that may persist can inhibit subsequent coupling. Following oxidation, the substrate and growing polynucleotide is optionally washed. In some instances, the step of oxidation is substituted with a sulfurization step to obtain polynucleotide phosphorothioates, wherein any capping steps can be performed after the sulfurization. Many reagents are capable of the efficient sulfur transfer, including but not limited to 3-(Dimethylaminomethylidene)amino)-3H-1,2,4-dithiazole-3-thione, DDTT, 3H-1,2-benzodithiol-3-one 1,1-dioxide, also known as Beaucage reagent, and N,N,N′N′-Tetraethylthiuram disulfide (TETD).

In order for a subsequent cycle of nucleoside incorporation to occur through coupling, the protected 5′ end of the substrate bound growing polynucleotide is removed so that the primary hydroxyl group is reactive with a next nucleoside phosphoramidite. In some instances, the protecting group is DMT and deblocking occurs with trichloroacetic acid in dichloromethane. Conducting detritylation for an extended time or with stronger than recommended solutions of acids may lead to increased depurination of solid support-bound polynucleotide and thus reduces the yield of the desired full-length product. Methods and compositions of the invention described herein provide for controlled deblocking conditions limiting undesired depurination reactions. In some cases, the substrate bound polynucleotide is washed after deblocking. In some cases, efficient washing after deblocking contributes to synthesized polynucleotides having a low error rate.

Methods for the synthesis of polynucleotides typically involve an iterating sequence of the following steps: application of a protected monomer to an actively functionalized surface (e.g., locus) to link with either the activated surface, a linker or with a previously deprotected monomer; deprotection of the applied monomer so that it is reactive with a subsequently applied protected monomer; and application of another protected monomer for linking. One or more intermediate steps include oxidation or sulfurization. In some cases, one or more wash steps precede or follow one or all of the steps.

Methods for phosphoramidite based polynucleotide synthesis comprise a series of chemical steps. In some instances, one or more steps of a synthesis method involve reagent cycling, where one or more steps of the method comprise application to the substrate of a reagent useful for the step. For example, reagents are cycled by a series of liquid deposition and vacuum drying steps. For substrates comprising three-dimensional features such as wells, microwells, channels and the like, reagents are optionally passed through one or more regions of the substrate via the wells and/or channels.

Polynucleotides synthesized using the methods and/or substrates described herein comprise at least about 20, 30, 40, 50, 60, 70, 75, 80, 90, 100, 120, 150, 200, 500 or more bases in length. In some instances, at least about 1 pmol, 10 pmol, 20 pmol, 30 pmol, 40 pmol, 50 pmol, 60 pmol, 70 pmol, 80 pmol, 90 pmol, 100 pmol, 150 pmol, 200 pmol, 300 pmol, 400 pmol, 500 pmol, 600 pmol, 700 pmol, 800 pmol, 900 pmol, 1 nmol, 5 nmol, 10 nmol, 100 nmol or more of an polynucleotide is synthesized within a locus. Methods for polynucleotide synthesis on a surface provided herein allow for synthesis at a fast rate. As an example, at least 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 70, 80, 90, 100, 125, 150, 175, 200 nucleotides per hour, or more are synthesized. Nucleotides include adenine, guanine, thymine, cytosine, uridine building blocks, or analogs/modified versions thereof. In some instances, libraries of polynucleotides are synthesized in parallel on a substrate. For example, a substrate comprising about or at least about 100; 1,000; 10,000; 100,000; 1,000,000; 2,000,000; 3,000,000; 4,000,000; or 5,000,000 resolved loci is able to support the synthesis of at least the same number of distinct polynucleotides, wherein a polynucleotide encoding a distinct sequence is synthesized on a resolved locus.

Various suitable methods are known for generating high density polynucleotide arrays. In an exemplary workflow, a substrate surface layer is provided. In the example, chemistry of the surface is altered in order to improve the polynucleotide synthesis process. Areas of low surface energy are generated to repel liquid while areas of high surface energy are generated to attract liquids. The surface itself may be in the form of a planar surface or contain variations in shape, such as protrusions or microwells which increase surface area. In the workflow example, high surface energy molecules selected serve a dual function of supporting DNA chemistry, as disclosed in International Patent Application Publication WO/2015/021080, which is herein incorporated by reference in its entirety.

In situ preparation of polynucleotide arrays is generated on a solid support and utilizes a single nucleotide extension process to extend multiple oligomers in parallel. A deposition device, such as a polynucleotide synthesizer, is designed to release reagents in a step wise fashion such that multiple polynucleotides extend, in parallel, one residue at a time to generate oligomers with a predetermined nucleic acid sequence. In some cases, polynucleotides are cleaved from the surface at this stage. Cleavage includes gas cleavage, e.g., with ammonia or methylamine.

### Substrates

Devices used as a surface for polynucleotide synthesis may be in the form of substrates which include, without limitation, homogenous array surfaces, patterned array surfaces, channels, beads, gels, and the like. Provided herein are substrates comprising a plurality of clusters, wherein each cluster comprises a plurality of loci that support the attachment and synthesis of polynucleotides. The term “locus” as used herein refers to a discrete region on a structure which provides support for polynucleotides encoding for a single predetermined sequence to extend from the surface. In some instances, a locus is on a two dimensional surface, e.g., a substantially planar surface. In some instances, a locus is on a three-dimensional surface, e.g., a well, microwell, channel, or post. In some instances, a surface of a locus comprises a material that is actively functionalized to attach to at least one nucleotide for polynucleotide synthesis, or preferably, a population of identical nucleotides for synthesis of a population of polynucleotides. In some instances, polynucleotide refers to a population of polynucleotides encoding for the same nucleic acid sequence. In some cases, a surface of a substrate is inclusive of one or a plurality of surfaces of a substrate. The average error rates for polynucleotides synthesized within a library described here using the systems and methods provided are often less than 1 in 1000, less than about 1 in 2000, less than about 1 in 3000 or less often without error correction.

Provided herein are surfaces that support the parallel synthesis of a plurality of polynucleotides having different predetermined sequences at addressable locations on a common support. In some instances, a substrate provides support for the synthesis of more than 50, 100, 200, 400, 600, 800, 1000, 1200, 1400, 1600, 1800, 2,000; 5,000; 10,000; 20,000; 50,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more non-identical polynucleotides. In some cases, the surfaces provide support for the synthesis of more than 50, 100, 200, 400, 600, 800, 1000, 1200, 1400, 1600, 1800, 2,000; 5,000; 10,000; 20,000; 50,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more polynucleotides encoding for distinct sequences. In some instances, at least a portion of the polynucleotides have an identical sequence or are configured to be synthesized with an identical sequence. In some instances, the substrate provides a surface environment for the growth of polynucleotides having at least 80, 90, 100, 120, 150, 175, 200, 225, 250, 275, 300, 325, 350, 375, 400, 425, 450, 475, 500 bases or more.

Provided herein are methods for polynucleotide synthesis on distinct loci of a substrate, wherein each locus supports the synthesis of a population of polynucleotides. In some cases, each locus supports the synthesis of a population of polynucleotides having a different sequence than a population of polynucleotides grown on another locus. In some instances, each polynucleotide sequence is synthesized with 1, 2, 3, 4, 5, 6, 7, 8, 9 or more redundancy across different loci within the same cluster of loci on a surface for polynucleotide synthesis. In some instances, the loci of a substrate are located within a plurality of clusters. In some instances, a substrate comprises at least 10, 500, 1000, 2000, 3000, 4000, 5000, 6000, 7000, 8000, 9000, 10000, 11000, 12000, 13000, 14000, 15000, 20000, 30000, 40000, 50000 or more clusters. In some instances, a substrate comprises more than 2,000; 5,000; 10,000; 100,000; 200,000; 300,000; 400,000; 500,000; 600,000; 700,000; 800,000; 900,000; 1,000,000; 1,100,000; 1,200,000; 1,300,000; 1,400,000; 1,500,000; 1,600,000; 1,700,000; 1,800,000; 1,900,000; 2,000,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; or 10,000,000 or more distinct loci. In some instances, a substrate comprises about 10,000 distinct loci. The amount of loci within a single cluster is varied in different instances. In some cases, each cluster includes 1, 2, 3, 4, 5, 6, 7, 8, 9, 10, 20, 30, 40, 50, 60, 70, 80, 90, 100, 120, 130, 150, 200, 300, 400, 500 or more loci. In some instances, each cluster includes about 50-500 loci. In some instances, each cluster includes about 100-200 loci. In some instances, each cluster includes about 100-150 loci. In some instances, each cluster includes about 109, 121, 130 or 137 loci. In some instances, each cluster includes about 19, 20, 61, 64 or more loci.

In some instances, the number of distinct polynucleotides synthesized on a substrate is dependent on the number of distinct loci available on the substrate. In some instances, the density of loci within a cluster of a substrate is at least or about 1, 10, 25, 50, 65, 75, 100, 130, 150, 175, 200, 300, 400, 500, 1,000 or more loci per mm². In some cases, a substrate comprises 10-500, 25-400, 50-500, 100-500, 150-500, 10-250, 50-250, 10-200, or 50-200 mm². In some instances, the distance between the centers of two adjacent loci within a cluster is from about 10-500, from about 10-200, or from about 10-100 um. In some instances, the distance between two centers of adjacent loci is greater than about 10, 20, 30, 40, 50, 60, 70, 80, 90 or 100 um. In some instances, the distance between the centers of two adjacent loci is less than about 200, 150, 100, 80, 70, 60, 50, 40, 30, 20 or 10 um. In some instances, each locus independently has a width of about 0.5, 1, 2, 3, 4, 5, 6, 7, 8, 9, 10, 20, 30, 40, 50, 60, 70, 80, 90 or 100 um. In some cases, each locus independently has a width of about 0.5-100, 0.5-50, 10-75, or 0.5-50 um.

In some instances, the density of clusters within a substrate is at least or about 1 cluster per 100 mm², 1 cluster per 10 mm², 1 cluster per 5 mm², 1 cluster per 4 mm², 1 cluster per 3 mm², 1 cluster per 2 mm², 1 cluster per 1 mm², 2 clusters per 1 mm², 3 clusters per 1 mm², 4 clusters per 1 mm², 5 clusters per 1 mm², 10 clusters per 1 mm², 50 clusters per 1 mm² or more. In some instances, a substrate comprises from about 1 cluster per 10 mm² to about 10 clusters per 1 mm². In some instances, the distance between the centers of two adjacent clusters is at least or about 50, 100, 200, 500, 1000, 2000, or 5000 um. In some cases, the distance between the centers of two adjacent clusters is between about 50-100, 50-200, 50-300, 50-500, or 100-2000 um. In some cases, the distance between the centers of two adjacent clusters is between about 0.05-50, 0.05-10, 0.05-5, 0.05-4, 0.05-3, 0.05-2, 0.1-10, 0.2-10, 0.3-10, 0.4-10, 0.5-10, 0.5-5, or 0.5-2 mm. In some cases, each cluster independently has a cross section of about 0.5 to 2, about 0.5 to 1, or about 1 to 2 mm. In some cases, each cluster independently has a cross section of about 0.5, 0.6, 0.7, 0.8, 0.9, 1, 1.1, 1.2, 1.3, 1.4, 1.5, 1.6, 1.7, 1.8, 1.9 or 2 mm. In some cases, each cluster independently has an interior cross section of about 0.5, 0.6, 0.7, 0.8, 0.9, 1, 1.1, 1.15, 1.2, 1.3, 1.4, 1.5, 1.6, 1.7, 1.8, 1.9 or 2 mm.

In some instances, a substrate is about the size of a standard 96 well plate, for example between about 100 to about 200 mm by between about 50 to about 150 mm. In some instances, a substrate has a diameter less than or equal to about 1000, 500, 450, 400, 300, 250, 200, 150, 100 or 50 mm. In some instances, the diameter of a substrate is between about 25-1000, 25-800, 25-600, 25-500, 25-400, 25-300, or 25-200 mm. In some instances, a substrate has a planar surface area of at least about 100; 200; 500; 1,000; 2,000; 5,000; 10,000; 12,000; 15,000; 20,000; 30,000; 40,000; 50,000 mm² or more. In some instances, the thickness of a substrate is between about 50-2000, 50-1000, 100-1000, 200-1000, or 250-1000 mm.

### Surface Materials

Substrates, devices, and reactors provided herein are fabricated from any variety of materials suitable for the methods, compositions, and systems described herein. In certain instances, substrate materials are fabricated to exhibit a low level of nucleotide binding. In some instances, substrate materials are modified to generate distinct surfaces that exhibit a high level of nucleotide binding. In some instances, substrate materials are transparent to visible and/or UV light. In some instances, substrate materials are sufficiently conductive, e.g., are able to form uniform electric fields across all or a portion of a substrate. In some instances, conductive materials are connected to an electric ground. In some instances, the substrate is heat conductive or insulated. In some instances, the materials are chemical resistant and heat resistant to support chemical or biochemical reactions, for example polynucleotide synthesis reaction processes. In some instances, a substrate comprises flexible materials. For flexible materials, materials can include, without limitation: nylon, both modified and unmodified, nitrocellulose, polypropylene, and the like. In some instances, a substrate comprises rigid materials. For rigid materials, materials can include, without limitation: glass; fuse silica; silicon, plastics (for example polytetraflouroethylene, polypropylene, polystyrene, polycarbonate, and blends thereof, and the like); metals (for example, gold, platinum, and the like). The substrate, solid support or reactors can be fabricated from a material selected from the group consisting of silicon, polystyrene, agarose, dextran, cellulosic polymers, polyacrylamides, polydimethylsiloxane (PDMS), and glass. The substrates/solid supports or the microstructures, reactors therein may be manufactured with a combination of materials listed herein or any other suitable material known in the art.

### Surface Architecture

Provided herein are substrates for the methods, compositions, and systems described herein, wherein the substrates have a surface architecture suitable for the methods, compositions, and systems described herein. In some instances, a substrate comprises raised and/or lowered features. One benefit of having such features is an increase in surface area to support polynucleotide synthesis. In some instances, a substrate having raised and/or lowered features is referred to as a three-dimensional substrate. In some cases, a three-dimensional substrate comprises one or more channels. In some cases, one or more loci comprise a channel. In some cases, the channels are accessible to reagent deposition via a deposition device such as a polynucleotide synthesizer. In some cases, reagents and/or fluids collect in a larger well in fluid communication with one or more channels. For example, a substrate comprises a plurality of channels corresponding to a plurality of loci within a cluster, and the plurality of channels are in fluid communication with one well of the cluster. In some methods, a library of polynucleotides is synthesized in a plurality of loci of a cluster.

Provided herein are substrates for the methods, compositions, and systems described herein, wherein the substrates are configured for polynucleotide synthesis. In some instances, the structure is configured to allow for controlled flow and mass transfer paths for polynucleotide synthesis on a surface. In some instances, the configuration of a substrate allows for the controlled and even distribution of mass transfer paths, chemical exposure times, and/or wash efficacy during polynucleotide synthesis. In some instances, the configuration of a substrate allows for increased sweep efficiency, for example by providing sufficient volume for growing a polynucleotide such that the excluded volume by the growing polynucleotide does not take up more than 50, 45, 40, 35, 30, 25, 20, 15, 14, 13, 12, 11, 10, 9, 8, 7, 6, 5, 4, 3, 2, 1%, or less of the initially available volume that is available or suitable for growing the polynucleotide. In some instances, a three-dimensional structure allows for managed flow of fluid to allow for the rapid exchange of chemical exposure.

Provided herein are substrates for the methods, compositions, and systems relating to enzymatic mediated nucleic acid assembly and polynucleotide synthesis described herein, wherein the substrates comprise structures configured for housing enzymatic reactions described herein. In some instances, segregation is achieved by physical structure. In some instances, segregation is achieved by differential functionalization of the surface generating active and passive regions for polynucleotide synthesis. In some instances, differential functionalization is achieved by alternating the hydrophobicity across the substrate surface, thereby creating water contact angle effects that cause beading or wetting of the deposited reagents. Employing larger structures can decrease splashing and cross-contamination of distinct polynucleotide synthesis locations with reagents of the neighboring spots. In some cases, a device, such as a polynucleotide synthesizer, is used to deposit reagents to distinct polynucleotide synthesis locations. Substrates having three-dimensional features are configured in a manner that allows for the synthesis of a large number of polynucleotides (e.g., more than about 10,000) with a low error rate (e.g., less than about 1:500, 1:1000, 1:1500, 1:2,000; 1:3,000; 1:5,000; or 1:10,000). In some cases, a substrate comprises features with a density of about or greater than about 1, 5, 10, 20, 30, 40, 50, 60, 70, 80, 100, 110, 120, 130, 140, 150, 160, 170, 180, 190, 200, 300, 400 or 500 features per mm².

A well of a substrate may have the same or different width, height, and/or volume as another well of the substrate. A channel of a substrate may have the same or different width, height, and/or volume as another channel of the substrate. In some instances, the diameter of a cluster or the diameter of a well comprising a cluster, or both, is between about 0.05-50, 0.05-10, 0.05-5, 0.05-4, 0.05-3, 0.05-2, 0.05-1, 0.05-0.5, 0.05-0.1, 0.1-10, 0.2-10, 0.3-10, 0.4-10, 0.5-10, 0.5-5, or 0.5-2 mm. In some instances, the diameter of a cluster or well or both is less than or about 5, 4, 3, 2, 1, 0.5, 0.1, 0.09, 0.08, 0.07, 0.06, or 0.05 mm. In some instances, the diameter of a cluster or well or both is between about 1.0 and about 1.3 mm. In some instances, the diameter of a cluster or well, or both is about 1.150 mm. In some instances, the diameter of a cluster or well, or both is about 0.08 mm. The diameter of a cluster refers to clusters within a two-dimensional or three-dimensional substrate.

In some instances, the height of a well is from about 20-1000, 50-1000, 100-1000, 200-1000, 300-1000, 400-1000, or 500-1000 um. In some cases, the height of a well is less than about 1000, 900, 800, 700, or 600 um.

In some instances, a substrate comprises a plurality of channels corresponding to a plurality of loci within a cluster, wherein the height or depth of a channel is 5-500, 5-400, 5-300, 5-200, 5-100, 5-50, or 10-50 um. In some cases, the height of a channel is less than 100, 80, 60, 40, or 20 um.

In some instances, the diameter of a channel, locus (e.g., in a substantially planar substrate) or both channel and locus (e.g., in a three-dimensional substrate wherein a locus corresponds to a channel) is from about 1-1000, 1-500, 1-200, 1-100, 5-100, or 10-100 um, for example, about 90, 80, 70, 60, 50, 40, 30, 20 or 10 um. In some instances, the diameter of a channel, locus, or both channel and locus is less than about 100, 90, 80, 70, 60, 50, 40, 30, 20 or 10 um. In some instances, the distance between the center of two adjacent channels, loci, or channels and loci is from about 1-500, 1-200, 1-100, 5-200, 5-100, 5-50, or 5-30, for example, about 20 um.

### Surface Modifications

Provided herein are methods for polynucleotide synthesis on a surface, wherein the surface comprises various surface modifications. In some instances, the surface modifications are employed for the chemical and/or physical alteration of a surface by an additive or subtractive process to change one or more chemical and/or physical properties of a substrate surface or a selected site or region of a substrate surface. For example, surface modifications include, without limitation, (1) changing the wetting properties of a surface, (2) functionalizing a surface, i.e., providing, modifying or substituting surface functional groups, (3) defunctionalizing a surface, i.e., removing surface functional groups, (4) otherwise altering the chemical composition of a surface, e.g., through etching, (5) increasing or decreasing surface roughness, (6) providing a coating on a surface, e.g., a coating that exhibits wetting properties that are different from the wetting properties of the surface, and/or (7) depositing particulates on a surface.

In some cases, the addition of a chemical layer on top of a surface (referred to as adhesion promoter) facilitates structured patterning of loci on a surface of a substrate. Exemplary surfaces for application of adhesion promotion include, without limitation, glass, silicon, silicon dioxide and silicon nitride. In some cases, the adhesion promoter is a chemical with a high surface energy. In some instances, a second chemical layer is deposited on a surface of a substrate. In some cases, the second chemical layer has a low surface energy. In some cases, surface energy of a chemical layer coated on a surface supports localization of droplets on the surface. Depending on the patterning arrangement selected, the proximity of loci and/or area of fluid contact at the loci are alterable.

In some instances, a substrate surface, or resolved loci, onto which nucleic acids or other moieties are deposited, e.g., for polynucleotide synthesis, are smooth or substantially planar (e.g., two-dimensional) or have irregularities, such as raised or lowered features (e.g., three-dimensional features). In some instances, a substrate surface is modified with one or more different layers of compounds. Such modification layers of interest include, without limitation, inorganic and organic layers such as metals, metal oxides, polymers, small organic molecules and the like.

In some instances, resolved loci of a substrate are functionalized with one or more moieties that increase and/or decrease surface energy. In some cases, a moiety is chemically inert. In some cases, a moiety is configured to support a desired chemical reaction, for example, one or more processes in a polynucleotide acid synthesis reaction. The surface energy, or hydrophobicity, of a surface is a factor for determining the affinity of a nucleotide to attach onto the surface. In some instances, a method for substrate functionalization comprises: (a) providing a substrate having a surface that comprises silicon dioxide; and (b) silanizing the surface using, a suitable silanizing agent described herein or otherwise known in the art, for example, an organofunctional alkoxysilane molecule. Methods and functionalizing agents are described in U.S. Pat. No. 5,474,796, which is herein incorporated by reference in its entirety.

In some instances, a substrate surface is functionalized by contact with a derivatizing composition that contains a mixture of silanes, under reaction conditions effective to couple the silanes to the substrate surface, typically via reactive hydrophilic moieties present on the substrate surface. Silanization generally covers a surface through self-assembly with organofunctional alkoxysilane molecules. A variety of siloxane functionalizing reagents can further be used as currently known in the art, e.g., for lowering or increasing surface energy. The organofunctional alkoxysilanes are classified according to their organic functions.

### Computer Systems

Any of the systems described herein, may be operably linked to a computer and may be automated through a computer either locally or remotely. In some instances, the methods and systems of the invention further comprise software programs on computer systems and use thereof. Accordingly, computerized control for the synchronization of the dispense/vacuum/refill functions such as orchestrating and synchronizing the material deposition device movement, dispense action and vacuum actuation are within the bounds of the invention. The computer systems may be programmed to interface between the user specified base sequence and the position of a material deposition device to deliver the correct reagents to specified regions of the substrate.

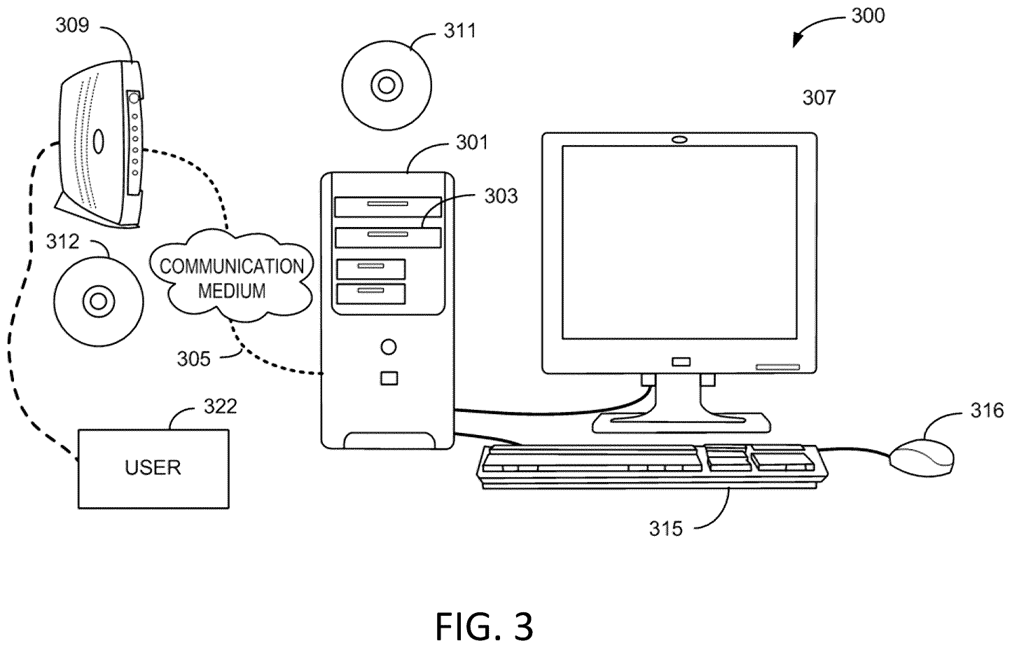

The computer system 300 illustrated in FIG. 3 may be understood as a logical apparatus that can read instructions from media 311 and/or a network port 305, which can optionally be connected to server 309 having fixed media 312. The system, such as shown in FIG. 3, can include a CPU 301, disk drives 303, optional input devices such as a keyboard 315 and/or mouse 316 and optional monitor 307. Data communication can be achieved through the indicated communication medium to a server at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present disclosure can be transmitted over such networks or connections for reception and/or review by a party 322 as illustrated in FIG. 3.

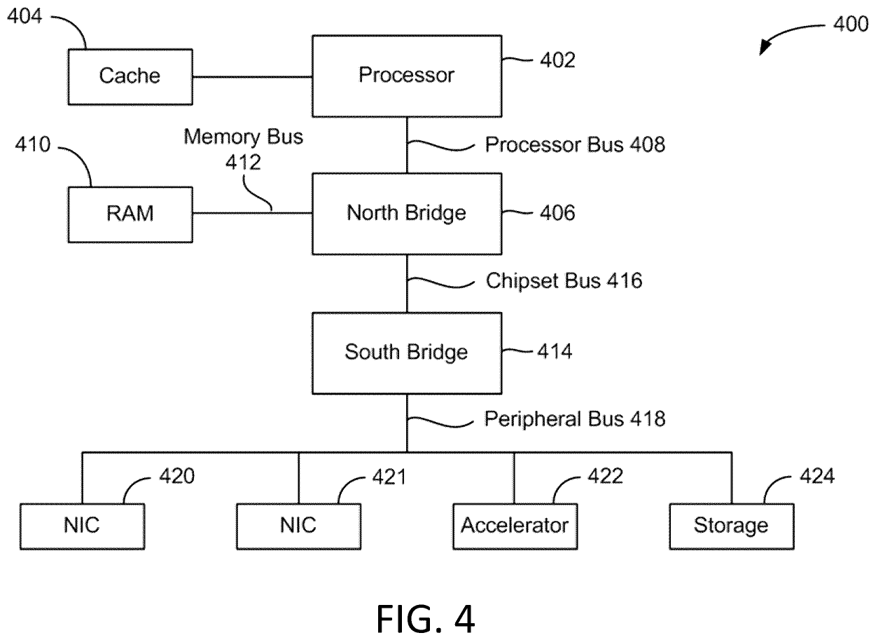

FIG. 4 is a block diagram illustrating architecture of a computer system 400 that can be used in connection with example embodiments of the present invention. As depicted in FIG. 4, the example computer system can include a processor 402 for processing instructions. Non-limiting examples of processors include: Intel® Xeon® processor, AMD Opteron™ processor, Samsung 32-bit RISC ARM 1176JZ(F)-S v1.0 processor, ARM Cortex-A8 Samsung S5PC100 processor, ARM Cortex-A8 Apple A4 processor, Marvell PXA 930 processor, or a functionally-equivalent processor. Multiple threads of execution can be used for parallel processing. In some instances, multiple processors or processors with multiple cores can also be used, whether in a single computer system, in a cluster, or distributed across systems over a network comprising a plurality of computers, cell phones, and/or personal data assistant devices.

As illustrated in FIG. 4, a high speed cache 404 can be connected to, or incorporated in, the processor 402 to provide a high speed memory for instructions or data that have been recently, or are frequently, used by processor 402. The processor 402 is connected to a north bridge 406 by a processor bus 408. The north bridge 406 is connected to random access memory (RAM) 410 by a memory bus 412 and manages access to the RAM 410 by the processor 402. The north bridge 406 is also connected to a south bridge 414 by a chipset bus 416. The south bridge 414 is, in turn, connected to a peripheral bus 418. The peripheral bus can be, for example, PCI, PCI-X, PCI Express, or other peripheral bus. The north bridge and south bridge are often referred to as a processor chipset and manage data transfer between the processor, RAM, and peripheral components on the peripheral bus 418. In some alternative architectures, the functionality of the north bridge can be incorporated into the processor instead of using a separate north bridge chip. In some instances, system 400 can include an accelerator card 422 attached to the peripheral bus 418. The accelerator can include field programmable gate arrays (FPGAs) or other hardware for accelerating certain processing. For example, an accelerator can be used for adaptive data restructuring or to evaluate algebraic expressions used in extended set processing.

Software and data are stored in external storage 424 and can be loaded into RAM 410 and/or cache 404 for use by the processor. The system 400 includes an operating system for managing system resources; non-limiting examples of operating systems include: Linux, Windows™, MACOS™, BlackBerry OS™, iOS™, and other functionally-equivalent operating systems, as well as application software running on top of the operating system for managing data storage and optimization in accordance with example embodiments of the present invention. In this example, system 400 also includes network interface cards (NICs) 420 and 421 connected to the peripheral bus for providing network interfaces to external storage, such as Network Attached Storage (NAS) and other computer systems that can be used for distributed parallel processing.

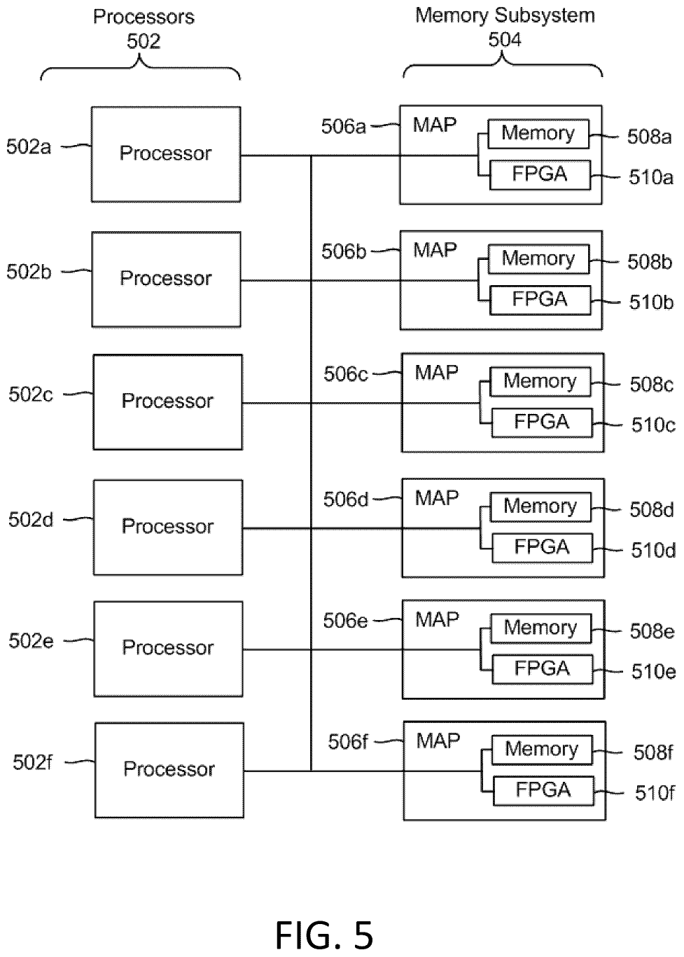

FIG. 5 is a block diagram of a multiprocessor computer system using a shared virtual address memory space in accordance with an example embodiment. The system includes a plurality of processors 502a-f that can access a shared memory subsystem 504. The system incorporates a plurality of programmable hardware memory algorithm processors (MAPs) 506a-f in the memory subsystem 504. Each MAP 506a-f can comprise a memory 508a-f and one or more field programmable gate arrays (FPGAs) 510a-f. The MAP provides a configurable functional unit and particular algorithms or portions of algorithms can be provided to the FPGAs 510a-f for processing in close coordination with a respective processor. For example, the MAPs can be used to evaluate algebraic expressions regarding the data model and to perform adaptive data restructuring in example embodiments. In this example, each MAP is globally accessible by all of the processors for these purposes. In one configuration, each MAP can use Direct Memory Access (DMA) to access an associated memory 508a-f, allowing it to execute tasks independently of, and asynchronously from, the respective microprocessor 502a-f. In this configuration, a MAP can feed results directly to another MAP for pipelining and parallel execution of algorithms.

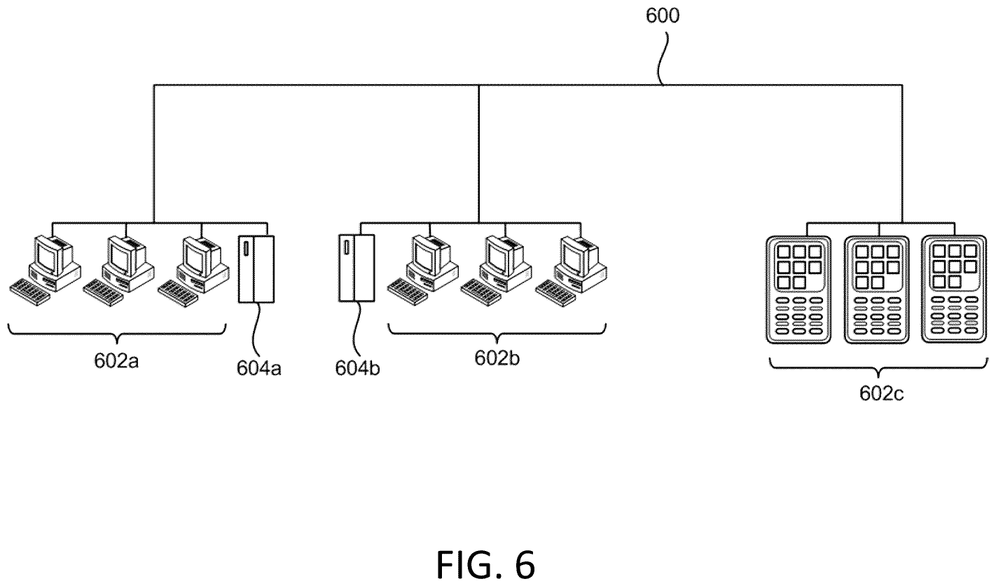

FIG. 6 is a diagram showing a network with a plurality of computer systems 602a and 602b, a plurality of cell phones and personal data assistants 602c, and Network Attached Storage (NAS) 604a and 604b. In example embodiments, systems 602a, 602b, and 602c can manage data storage and optimize data access for data stored in Network Attached Storage (NAS) 604a and 604b. A mathematical model can be used for the data and be evaluated using distributed parallel processing across computer systems 602a and 602b, and cell phone and personal data assistant systems 602c. Computer systems 602a and 602b, and cell phone and personal data assistant systems 602c can also provide parallel processing for adaptive data restructuring of the data stored in Network Attached Storage (NAS) 604a and 604b. FIG. 6 illustrates an example only, and a wide variety of other computer architectures and systems can be used in conjunction with the various embodiments of the present invention. For example, a blade server can be used to provide parallel processing. Processor blades can be connected through a back plane to provide parallel processing. Storage can also be connected to the back plane or as Network Attached Storage (NAS) through a separate network interface. In some instances, processors can maintain separate memory spaces and transmit data through network interfaces, back plane or other connectors for parallel processing by other processors. In some instances, some or all of the processors can use a shared virtual address memory space.

Any of the systems described herein may comprise sequence information stored on non-transitory computer readable storage media. In some instances, any of the systems described herein comprise a computer input file. In some instances, the computer input file comprises sequence information. In some instances, the computer input file comprises instructions for synthesis of a plurality of polynucleotide sequences. In some instances, the instructions are received by a computer. In some instances, the instructions are processed by the computer. In some instances, the instructions are transmitted to a material deposition device. In some instances, the non-transitory computer readable storage media is encoded with a program including instructions executable by the operating system of an optionally networked digital processing device. In some instances, a computer readable storage medium is a tangible component of a digital processing device. In some instances, a computer readable storage medium is optionally removable from a digital processing device. In some instances, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some instances, the program and instructions are permanently, substantially permanently, semi-permanently, or non-transitorily encoded on the media.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Functionalization of a Substrate Surface

A substrate was functionalized to support the attachment and synthesis of a library of polynucleotides. The substrate surface was first wet cleaned using a piranha solution comprising 90% H₂SO₄ and 10% H₂O₂ for 20 minutes. The substrate was rinsed in several beakers with deionized water, held under a deionized water gooseneck faucet for 5 min, and dried with N₂. The substrate was subsequently soaked in NH₄OH (1:100; 3 mL:300 mL) for 5 min, rinsed with DI water using a handgun, soaked in three successive beakers with deionized water for 1 min each, and then rinsed again with deionized water using the handgun. The substrate was then plasma cleaned by exposing the substrate surface to O₂. A SAMCO PC-300 instrument was used to plasma etch O₂ at 250 watts for 1 min in downstream mode.

The cleaned substrate surface was actively functionalized with a solution comprising N-(3-triethoxysilylpropyl)-4-hydroxybutyramide using a YES-1224P vapor deposition oven system with the following parameters: 0.5 to 1 torr, 60 min, 70° C., 135° C. vaporizer. The substrate surface was resist coated using a Brewer Science 200X spin coater. SPR™ 3612photoresist was spin coated on the substrate at 2500 rpm for 40 sec. The substrate was pre-baked for 30 min at 90° C. on a Brewer hot plate. The substrate was subjected to photolithography using a Karl Suss MA6 mask aligner instrument. The substrate was exposed for 2.2 sec and developed for 1 min in MSF 26A. Remaining developer was rinsed with the handgun and the substrate soaked in water for 5 min. The substrate was baked for 30 min at 100° C. in the oven, followed by visual inspection for lithography defects using a Nikon L200. A cleaning process was used to remove residual resist using the SAMCO PC-300 instrument to O₂ plasma etch at 250 watts for 1 min.

The substrate surface was passively functionalized with a 100 μL solution of perfluorooctyltrichlorosilane mixed with 10 μL light mineral oil. The substrate was placed in a chamber, pumped for 10 min, and then the valve was closed to the pump and left to stand for 10 min. The chamber was vented to air. The substrate was resist stripped by performing two soaks for 5 min in 500 mL NMP at 70° C. with ultrasonication at maximum power (9 on Crest system). The substrate was then soaked for 5 min in 500 mL isopropanol at room temperature with ultrasonication at maximum power. The substrate was dipped in 300 mL of 200 proof ethanol and blown dry with N₂. The functionalized surface was activated to serve as a support for polynucleotide synthesis.

### Example 2: Synthesis of a 50-mer Sequence on an Oligonucleotide Synthesis Device

A two dimensional oligonucleotide synthesis device was assembled into a flowcell, which was connected to a flowcell (Applied Biosystems (“ABI394 DNA Synthesizer”)). The two-dimensional oligonucleotide synthesis device was uniformly functionalized with N-(3-TRIETHOXYSILYLPROPYL)-4-HYDROXYBUTYRAMIDE (Gelest) was used to synthesize an exemplary polynucleotide of 50 bp (“50-mer polynucleotide”) using polynucleotide synthesis methods described herein.

The sequence of the 50-mer was as described in SEQ ID NO.: 1.5′AGACAATCAACCATTTGGGGGGACAGCCTTGACCTCTAGACTTCGGCAT##TTTTT TTTTT3′ (SEQ ID NO.: 1), where #denotes Thymidine-succinyl hexamide CED phosphoramidite (CLP-2244 from ChemGenes), which is a cleavable linker enabling the release of polynucleotides from the surface during deprotection.

The synthesis was done using standard DNA synthesis chemistry (coupling, capping, oxidation, and deblocking) according to the protocol in Table 3 and ABI394 DNA Synthesizer.

TABLE 3
Synthesis Protocol

| General DNA Synthesis | | |
|---|---|---|
| Process Name | Process Step | Time (sec) |
| WASH (Acetonitrile Wash | Acetonitrile System Flush | 4 |
| Flow) | Acetonitrile to Flowcell | 23 |
| | N2 System Flush | 4 |
| | Acetonitrile System Flush | 4 |
| DNA BASE ADDITION | Activator Manifold Flush | 2 |
| (Phosphoramidite + | Activator to Flowcell | 6 |
| Activator Flow) | Activator + | 6 |
| | Phosphoramidite to | |
| | Flowcell | |
| | Activator to Flowcell | 0.5 |
| | Activator + | 5 |
| | Phosphoramidite to | |
| | Flowcell | |
| | Activator to Flowcell | 0.5 |
| | Activator + | 5 |
| | Phosphoramidite to | |
| | Flowcell | |
| | Activator to Flowcell | 0.5 |
| | Activator + | 5 |
| | Phosphoramidite to | |
| | Flowcell | |
| | Incubate for 25 sec | 25 |
| WASH (Acetonitrile Wash | Acetonitrile System Flush | 4 |
| Flow) | Acetonitrile to Flowcell | 15 |
| | N2 System Flush | 4 |
| | Acetonitrile System Flush | 4 |
| DNA BASE ADDITION | Activator Manifold Flush | 2 |
| (Phosphoramidite + | Activator to Flowcell | 5 |
| Activator Flow) | Activator + | 18 |
| | Phosphoramidite to | |
| | Flowcell | |
| | Incubate for 25 sec | 25 |
| WASH (Acetonitrile Wash | Acetonitrile System Flush | 4 |
| Flow) | Acetonitrile to Flowcell | 15 |
| | N2 System Flush | 4 |
| | Acetonitrile System Flush | 4 |
| CAPPING (CapA + B, 1:1, | CapA + B to Flowcell | 15 |
| Flow) | | |
| WASH (Acetonitrile Wash | Acetonitrile System Flush | 4 |
| Flow) | Acetonitrile to Flowcell | 15 |
| | Acetonitrile System Flush | 4 |
| OXIDATION (Oxidizer | Oxidizer to Flowcell | 18 |
| Flow) | | |
| WASH (Acetonitrile Wash | Acetonitrile System Flush | 4 |
| Flow) | N2 System Flush | 4 |
| | Acetonitrile System Flush | 4 |
| | Acetonitrile to Flowcell | 15 |
| | Acetonitrile System Flush | 4 |
| | Acetonitrile to Flowcell | 15 |
| | N2 System Flush | 4 |
| | Acetonitrile System Flush | 4 |
| | Acetonitrile to Flowcell | 23 |
| | N2 System Flush | 4 |
| | Acetonitrile System Flush | 4 |
| DEBLOCKING (Deblock | Deblock to Flowcell | 36 |
| Flow) | | |
| WASH (Acetonitrile Wash | Acetonitrile System Flush | 4 |
| Flow) | N2 System Flush | 4 |
| | Acetonitrile System Flush | 4 |
| | Acetonitrile to Flowcell | 18 |
| | N2 System Flush | 4.13 |
| | Acetonitrile System Flush | 4.13 |
| | Acetonitrile to Flowcell | 15 |

The phosphoramidite/activator combination was delivered similar to the delivery of bulk reagents through the flowcell. No drying steps were performed as the environment stays “wet” with reagent the entire time.

The flow restrictor was removed from the ABI394 DNA Synthesizer to enable faster flow. Without flow restrictor, flow rates for amidites (0.1M in ACN), Activator, (0.25M Benzoylthiotetrazole (“BTT”; 30-3070-xx from GlenResearch) in ACN), and Ox (0.02M I2 in 20% pyridine, 10% water, and 70% THF) were roughly ˜100 uL/sec, for acetonitrile (“ACN”) and capping reagents (1:1 mix of CapA and CapB, wherein CapA is acetic anhydride in THF/Pyridine and CapB is 16% 1-methylimidizole in THF), roughly ˜200 uL/sec, and for Deblock (3% dichloroacetic acid in toluene), roughly ˜300 uL/sec (compared to ˜50 uL/sec for all reagents with flow restrictor). The time to completely push out Oxidizer was observed, the timing for chemical flow times was adjusted accordingly and an extra ACN wash was introduced between different chemicals. After polynucleotide synthesis, the chip was deprotected in gaseous ammonia overnight at 75 psi. Five drops of water were applied to the surface to recover polynucleotides. The recovered polynucleotides were then analyzed on a BioAnalyzer small RNA chip (data not shown).

### Example 3: Synthesis of a 100-mer Sequence on an Oligonucleotide Synthesis Device

The same process as described in Example 2 for the synthesis of the 50-mer sequence was used for the synthesis of a 100-mer polynucleotide (“100-mer polynucleotide”; 5′ CGGGATCCTTATCGTCATCGTCGTACAGATCCCGACCCATTTGCTGTCCACCAGTCAT GCTAGCCATACCATGATGATGATGATGATGAGAACCCCGCAT##TTTTTTTTTT3′,where #denotes Thymidine-succinyl hexamide CED phosphoramidite (CLP-2244 from ChemGenes); SEQ ID NO.: 2) on two different silicon chips, the first one uniformly functionalized with N-(3-TRIETHOXYSILYLPROPYL)-4-HYDROXYBUTYRAMIDE and the second one functionalized with 5/95 mix of 11-acetoxyundecyltriethoxysilane and n-decyltriethoxysilane, and the polynucleotides extracted from the surface were analyzed on a BioAnalyzer instrument (data not shown).

All ten samples from the two chips were further PCR amplified using a forward (5′ATGCGGGGTTCTCATCATC3′; SEQ ID NO.: 3) and a reverse (5′CGGGATCCTTATCGTCATCG3′; SEQ ID NO.: 4) primer in a 50 uL PCR mix (25 uL NEB Q5 mastermix, 2.5 uL 10 uM Forward primer, 2.5 uL 10 uM Reverse primer, 1 uL polynucleotide extracted from the surface, and water up to 50 uL) using the following thermalcycling program:
98° C., 30 sec
98° C., 10 sec; 63° C., 10 sec; 72° C., 10 sec; repeat 12 cycles
72° C., 2 min

The PCR products were also run on a BioAnalyzer (data not shown), demonstrating sharp peaks at the 100-mer position. Next, the PCR amplified samples were cloned, and Sanger sequenced. Table 4 summarizes the results from the Sanger sequencing for samples taken from spots 1-5 from chip 1 and for samples taken from spots 6-10 from chip 2.

TABLE 4
Sequencing Results

| Spot | Error rate | Cycle efficiency |
|---|---|---|
| 1 | 1/763 bp | 99.87% |
| 2 | 1/824 bp | 99.88% |
| 3 | 1/780 bp | 99.87% |
| 4 | 1/429 bp | 99.77% |
| 5 | 1/1525 bp | 99.93% |
| 6 | 1/1615 bp | 99.94% |
| 7 | 1/531 bp | 99.81% |
| 8 | 1/1769 bp | 99.94% |
| 9 | 1/854 bp | 99.88% |
| 10 | 1/1451 bp | 99.93% |

Thus, the high quality and uniformity of the synthesized polynucleotides were repeated on two chips with different surface chemistries. Overall, 89%, corresponding to 233 out of 262 of the 100-mers that were sequenced were perfect sequences with no errors. Table 5 summarizes error characteristics for the sequences obtained from the polynucleotides samples from spots 1-10.

TABLE 5
Error Characteristics

| Sample ID/ | OSA_ | OSA_ | OSA_ | OSA_ | OSA_ | OSA_ | OSA_ | OSA_ | OSA_ | OSA_ |
|---|---|---|---|---|---|---|---|---|---|---|
| Spot no. | 0046/1 | 0047/2 | 0048/3 | 0049/4 | 0050/5 | 0051/6 | 0052/7 | 0053/8 | 0054/9 | 0055/10 |
| Total Sequences | 32 | 32 | 32 | 32 | 32 | 32 | 32 | 32 | 32 | 32 |
| Sequencing | 25 of 28 | 27 of 27 | 26 of 30 | 21 of 23 | 25 of 26 | 29 of 30 | 27 of 31 | 29 of 31 | 28 of 29 | 25 of 28 |
| Quality | | | | | | | | | | |
| Oligo Quality | 23 of 25 | 25 of 27 | 22 of 26 | 18 of 21 | 24 of 25 | 25 of 29 | 22 of 27 | 28 of 29 | 26 of 28 | 20 of 25 |
| ROI Match | 2500 | 2698 | 2561 | 2122 | 2499 | 2666 | 2625 | 2899 | 2798 | 2348 |
| Count | | | | | | | | | | |
| ROI Mutation | 2 | 2 | 1 | 3 | 1 | 0 | 2 | 1 | 2 | 1 |
| ROI Multi Base | 0 | 0 | 0 | 0 | 0 | 0 | 0 | 0 | 0 | 0 |
| Deletion | | | | | | | | | | |
| ROI Small | 1 | 0 | 0 | 0 | 0 | 0 | 0 | 0 | 0 | 0 |
| Insertion | | | | | | | | | | |
| ROI Single Base | 0 | 0 | 0 | 0 | 0 | 0 | 0 | 0 | 0 | 0 |
| Deletion | | | | | | | | | | |
| Large Deletion | 0 | 0 | 1 | 0 | 0 | 1 | 1 | 0 | 0 | 0 |
| Count | | | | | | | | | | |
| Mutation: G > A | 2 | 2 | 1 | 2 | 1 | 0 | 2 | 1 | 2 | 1 |
| Mutation: T > C | 0 | 0 | 0 | 1 | 0 | 0 | 0 | 0 | 0 | 0 |
| ROI Error Count | 3 | 2 | 2 | 3 | 1 | 1 | 3 | 1 | 2 | 1 |
| ROI Error Rate | Err: ~1 | Err: ~1 | Err: ~1 | Err: ~1 | Err: ~1 | Err: ~1 | Err: ~1 | Err: ~1 | Err: ~1 | Err: ~1 |
| | in 834 | in 1350 | in 1282 | in 708 | in 2500 | in 2667 | in 876 | in 2900 | in 1400 | in 2349 |
| ROI Minus | MP Err: | MP Err: | MP Err: | MP Err: | MP Err: | MP Err: | MP Err: | MP Err: | MP Err: | MP Err: |
| Primer Error | ~1 in | ~1 in | ~1 in | ~1 in | ~1 in | ~1 in | ~1 in | ~1 in | ~1 in | ~1 in |
| Rate | 763 | 824 | 780 | 429 | 1525 | 1615 | 531 | 1769 | 854 | 1451 |

### Example 4. Flap Endonuclease Mediated Nucleic Acid Assembly

### Flap Endonuclease Mediated Nucleic Acid Assembly Reaction

A flap endonuclease mediated nucleic acid assembly reaction was prepared. Water, dNTP's (New England Biolabs), Ampligase buffer (Epicentre), ExoIII (New England Biolabs), Phusion (New England Biolabs), Ampligase (Epicentre), and Fen1 (New England Biolabs) according to the concentrations in Table 6 below were combined and aliquoted into 96 well plates. DNA and vector were added in the concentrations as indicated in Table 6. Plates were sealed, mixed for 30 seconds at 1000 rpm, and briefly centrifuged. Plates were incubated at 50° C. with 105° C. heated lid for 30 minutes and then cooled to 4° C. Reactions were diluted 1:5 in 40 uL of cold buffer.

TABLE 6
Reaction Concentrations

| Reagent | Final Concentration | |
|---|---|---|
| Vector | 4 | nM |
| Gene Fragment 1 | 4 | nM |
| dNTP | .2 | mM |
| 10X Ampligase buffer | 1X | |
| ExoIII | 10 | U |
| Phusion | 0.2 | U |
| Ampligase | 1 | U |
| Fen1 | 3.2 | U |

| | |
|---|---|
| Water | Remaining water up to 10 uL |

### In Vitro PCR Assay

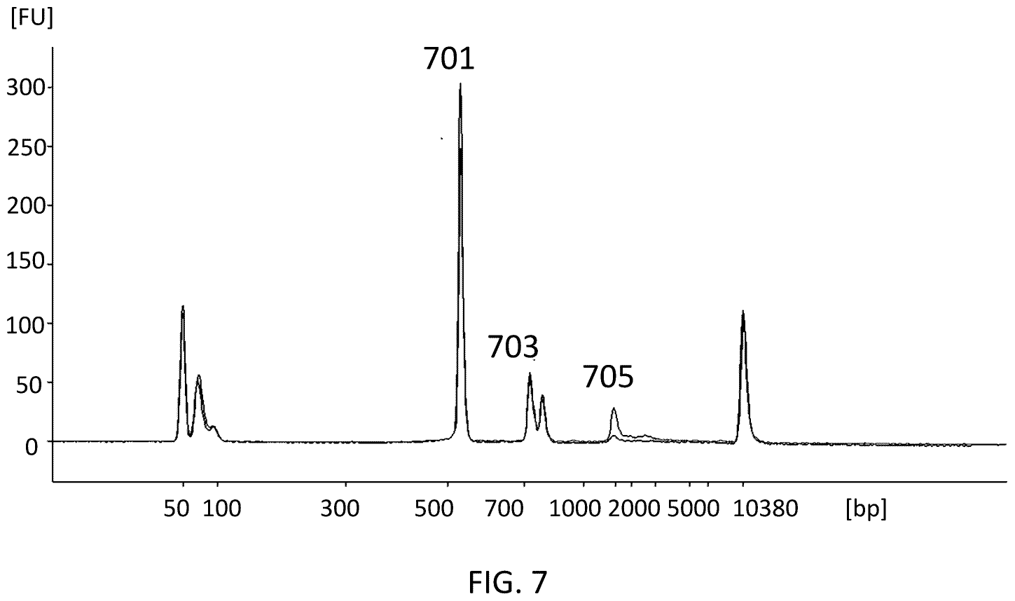

Following preparation of the flap endonuclease mediated nucleic acid assembly reaction, PCR amplification was performed. A 25 uL PCR reaction was performed according to reaction conditions in Table 7 below and amplified using thermocycler conditions according to Table 8. PCR products were analyzed on a BioAnalyzer (Agilent) (FIG. 7). Unspecified background band 701, circular vector 703, and assembled DNA inserted into the vector 705 were detected.

TABLE 7
PCR Reaction Conditions

| Reagent | Reaction Volume (uL) |
|---|---|
| Water | 16.5 |
| 10X Thermopol Buffer | 2.5 |
| 10 mM dNTP | 0.5 |
| PCR Primer 1 100 uM | 0.125 |
| PCR Primer 2 100 uM | 0.125 |
| Diluted Reaction Product | 5 |
| Taq polymerase | 0.25 |
| Total Volume | 25 |

TABLE 8
Thermocycler Conditions

| Temperature | Time | Number of Cycles |
|---|---|---|
| 95° C. | 30 seconds | 1 |
| 95° C. | 20 seconds | 25 |
| 55° C. | 20 seconds | |
| 68° C. | 3 minutes | |
| 68° C. | 5 minutes | 1 |
| 4° C. | Indefinite | |

### Transformation

Following the PCR reaction, 2 uL of the diluted reactions were electroporated into 20 uL of electrocompetent cells 10G (Lucigen). Cells were recovered with 600 uL pre-warmed Lucigen recovery media. Samples were serially diluted 1:2 into Lucigen recovery media and 7 uL was spotted onto a Lennox+Carb plate. Plates were grown overnight (about 16 hours) at 37° C. overnight. The reaction efficiency was determined by counting colonies to determine Colony Forming Units (CFU) using the CFU formula: (#of colonies*total reaction volume)/(plating volume*dilution factor).

The colony counts of E. coli transformants is seen in Table 9. The fold change was determined by the number of colonies with insert (1× dilution) compared to the number of colonies with no insert.

TABLE 9
Colony Counts

| | | Number of | Number of | |
|---|---|---|---|---|
| | Number of | Colonies with | Colonies with | |
| | Colonies with | Insert (1X | Insert (10X | |
| Reaction | No Insert | Dilution) | Dilution) | Fold Change |
| 50 C.- | 19 | 23 | | 1.2 |
| Exo | | | | |
| 50° C. | 58 | 929 | 100 | 16 |
| 10 U ExoIII | | | | |
| 65° C. | 25 | 33 | | 1.3 |
| 10 U ExoIII | | | | |

A correlation of the in vitro PCR assay (FIG. 7) to the number of E. coli transformants as a read out of assembled constructs (Table 10) was observed. Specifically a linearized vector was assembled with 1 DNA insert containing 40 base pair of homology to each other. Colony formulation in E coli correlates to the re-circularization of the vector after being assembled with the insert. The peak size from the in vitro PCR assay correlated to the number of E coli colony counts after transformation. The negative control reactions lacked DNA insert and showed low levels of background colonies.

### Example 5. Flap Endonuclease Mediated Nucleic Acid Assembly with Three Gene Fragments

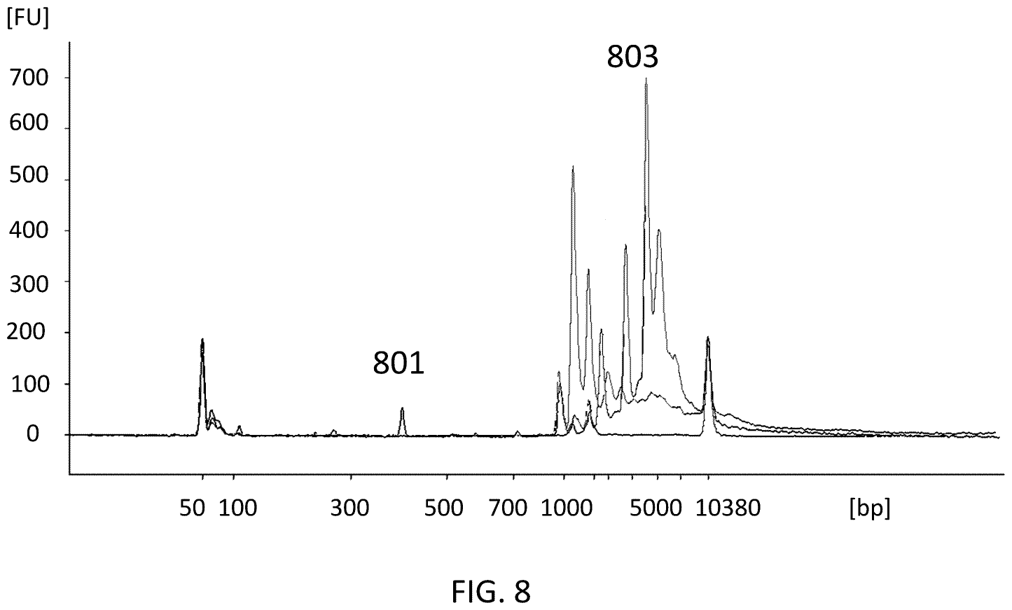

A flap endonuclease mediated nucleic acid assembly reaction was performed similar to Example 4. Three DNA fragments were inserted into a vector. Following preparation of the flap endonuclease mediated nucleic acid assembly reaction, PCR amplification was performed similar to Example 4. PCR products were analyzed on a BioAnalyzer (Agilent) (FIG. 8). Re-circularized digested vector 801 and assembled DNA inserted in the vector 803 were detected.

### Example 6. Flap Endonuclease Mediated Nucleic Acid Assembly With Varying Concentrations of Fen1 and ExoIII

A flap endonuclease mediated nucleic acid assembly reaction was prepared similarly to Example 4. Varying conditions were tested as seen in Table 10.

TABLE 10
Reaction Conditions

| Sample | Reaction Condition |
|---|---|
| 1 | Baseline |
| 2 | No Ampligase |
| 3 | No Phusion |
| 4 | No Fen1 |
| 5 | 2X Fen1 (6.4 U Fen1) |
| 6 | 0.5X Fen1(1.7 U Fen1) |
| 7 | 2X Fen1 (6.4 U Fen1) |
| | 2X ExoIII (20 U ExoIII) |
| 8 | 0.5X Fen1(1.7 U Fen1) |
| | 0.5X ExoIII (5 U ExoIII) |
| 9 | 0.25X Fen1 (0.8 U Fen1) |
| | 0.25X ExoIII (2.5 U ExoIII) |
| 10 | Reaction conditions from in vitro |
| | recombination cloning |

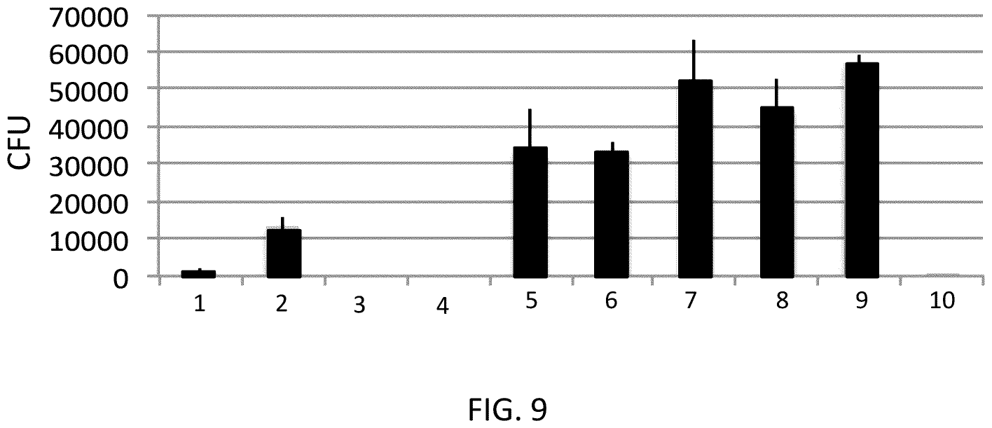

The number of colonies was counted for each sample as seen in FIG. 9. Data showed that colony counts with titrated Fen1 and ExoIII were significantly higher than baseline and correlated to higher amplification products in the in vitro PCR assay.

Colonies were isolated, and assemblies were assayed by colony PCR. Reactions were run on a Fragment Analyzer (capillary gel electrophoresis). Proper assemblies were determined by size of PCR product (data not shown).

Isolated colonies were grown overnight and miniprepped to isolate the assembled vector DNA. Samples were analyzed by next-generation sequencing. The varying Fen1 and ExoIII concentrations resulted in varying amount of correctly assembled constructs as seen in Table 11. All of the universal primer flanking sequences were removed in the passed samples. In the 7/10 samples that had the proper size in the colony PCR, all 7 passed NGS at the homology sites.

TABLE 11
Colony Data

| | Colonies | % Correct Size by | % Passed NGS |
|---|---|---|---|
| Reaction | Picked | Colony PCR | Analysis |
| Baseline − insert | 1 | 0 | 0 |
| Baseline + insert | 7 | 0 | 0 |
| 2X Fen1 (6.4 U Fen1) | 10 | 70 | 70 |
| 2X Fen1 (6.4 U Fen1) | 10 | 0 | 0 |
| 2X ExoIII (20 U ExoIII) | | | |
| 0.5X Fen1(1.7 U Fen1) | 10 | 50 | 30-50 |
| 0.5X ExoIII (5 U ExoIII) | | | |
| 0.25X Fen1 (0.8 U Fen1) | 10 | 20 | 20 |
| 0.25X ExoIII (2.5 U | | | |
| ExoIII) | | | |

### Example 7. Flap Endonuclease Mediated Nucleic Acid Assembly With Varying Concentrations of Enzymes

A flap endonuclease mediated nucleic acid assembly reaction was prepared similarly to Example 4. Three gene fragments were inserted into a vector. Experiments were performed using reaction conditions according to Table 12 below as a baseline and variations on Phusion, Ampligase, ExoIII, and Fen1 concentrations. The concentrations of Phusion, Ampligase, ExoIII, and Fen1 used are shown in Table 13.

TABLE 12
Reaction Conditions

| Reagent | Final Concentration | |
|---|---|---|
| Vector | 4 | nM |
| Gene Fragment | 4 | nM |
| dNTP | 0.2 | mM |
| 10X Ampligase buffer | 1X | |
| ExoIII | 1 | U |
| Phusion | 0.2 U or 0.1 U | |
| Ampligase | 1 | U |
| Fen1 | 0.32 | U |

| | |
|---|---|
| Water | Remaining water up to 10 uL |

TABLE 13
Enzyme Concentrations

| | Reaction Condition | |
|---|---|---|
| 1 | 0.32 | U Fen1 |
| | 1 | U ExoIII |
| | 0.2 | U Phusion |
| | 1 | U Ampligase |
| 2 | 0.32 | U Fen1 |
| | 1 | U ExoIII |
| | 0.1 | U Phusion |
| | 0.5 | U Ampligase |
| 3 | 0.32 | U Fen1 |
| | 1 | U ExoIII |
| | 0.1 | U Phusion |
| | 1.0 | U Ampligase |
| 4 | 0.32 | U Fen1 |
| | 1 | U ExoIII |
| | 0.05 | U Phusion |
| | 1.0 | U Ampligase |
| 5 | 0.32 | U Fen1 |
| | 1.5 | U ExoIII |
| | 0.2 | U Phusion |
| | 1.0 | U Ampligase |
| 6 | 4.8 | U Fen1 |
| | 1.0 | U ExoIII |
| | 0.2 | U Phusion |
| | 1.0 | U Ampligase |
| 7 | 0.32 | U Fen1 |
| | 0.5 | U ExoIII |
| | 0.05 | U Phusion |
| | 1.0 | U Ampligase |
| 8 | 0.32 | U Fen1 |
| | 1.0 | U ExoIII |
| | 0.1 | U Phusion |
| | 0.1 | U Ampligase |
| 9 | 0.32 | U Fen1 |
| | 1.0 | U ExoIII |
| | 0.1 | U Phusion |
| | 0.25 | U Ampligase |
| 10 | 0.32 | U Fen1 |
| | 1.0 | U ExoIII |
| | 0.2 | U Phusion |
| | 0.5 | U Ampligase |
| 11 | 0.32 | U Fen1 |
| | 1.0 | U ExoIII |
| | 0.2 | U Phusion |
| | 0.25 | U Ampligase |
| 12 | 0.32 | U Fen1 |
| | 0.5 | U ExoIII |
| | 0.1 | U Phusion |
| | 1.0 | U Ampligase |
| 13 | 3.2 | U Fen1 |
| | 1.0 | U ExoIII |
| | 0.2 | U Phusion |
| | 1.0 | U Ampligase |
| 14 | 0.32 | U Fen1 |
| | 0.5 | U ExoIII |
| | 0.2 | U Phusion |
| | 1.0 | U Ampligase |
| 15 | 0.32 | U Fen1 |
| | 1.5 | U ExoIII |
| | 0.1 | U Phusion |
| | 1.0 | U Ampligase |
| 16 | 0.32 | U Fen1 |
| | 1.5 | U ExoIII |
| | 0.05 | U Phusion |
| | 1.0 | U Ampligase |
| 17 | 3.2 | U Fen1 |
| | 0.5 | U ExoIII |
| | 0.2 | U Phusion |
| | 0.5 | U Ampligase |
| 18 | 3.2 | U Fen1 |
| | 1.0 | U ExoIII |
| | 0.2 | U Phusion |
| | 0.5 | U Ampligase |
| 19 | 3.2 | U Fen1 |
| | 1.0 | U ExoIII |
| | 0.2 | U Phusion |
| | 0 | U Ampligase |
| 20 | 4.8 | U Fen1 |
| | 0.5 | U ExoIII |
| | 0.2 | U Phusion |
| | 1.0 | U Ampligase |
| 21 | 0.32 | U Fen1 |
| | 1.5 | U ExoIII |
| | 0.5 | U Phusion |
| | 1.0 | U Ampligase |
| 22 | 3.2 | U Fen1 |
| | 0.5 | U ExoIII |
| | 0.2 | U Phusion |
| | 1.0 | U Ampligase |
| 23 | 0.32 | U Fen1 |
| | 1.0 | U ExoIII |
| | 0.2 | U Phusion |
| | 0.1 | U Ampligase |
| 24 | 0.32 | U Fen1 |
| | 0.5 | U ExoIII |
| | 0.5 | U Phusion |
| | 1.0 | U Ampligase |
| 25 | 0.32 | U Fen1 |
| | 1.0 | U ExoIII |
| | 0.5 | U Phusion |
| | 1.0 | U Ampligase |
| 26 | 3.2 | U Fen1 |
| | 10.0 | U ExoIII |
| | 0.2 | U Phusion |
| | 1.0 | U Ampligase |
| 27 | 3.2 | U Fen1 |
| | 5.0 | U ExoIII |
| | 0.2 | U Phusion |
| | 1.0 | U Ampligase |

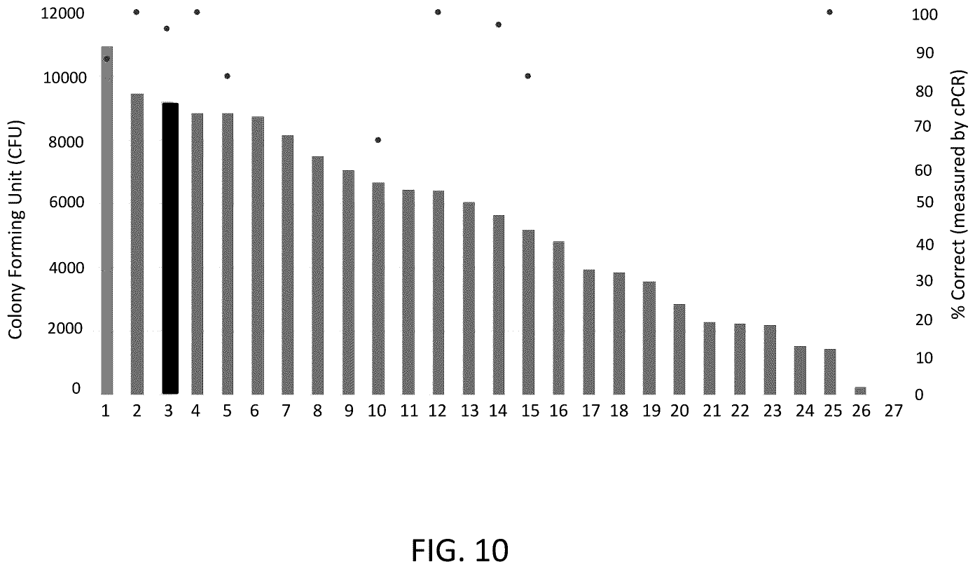

Referring to FIG. 10, the average colony forming unit (CFU) (gray bars) were measured for each of the different enzyme ratios. The percentage of correct assembly was also measured (black circles) as determined by colony PCR (cPCR). The data presented is an average of the results.

Using the different enzyme ratios, there was increased number of CFUs and improved percentage of correct assembly (FIG. 10). Reaction condition comprising 0.32 U Fen1, 1 U ExoIII, 0.2 U Phusion, and 1 U Ampligase resulted in the highest increase in the number of CFUs as compared to other reaction conditions and a percentage of correct assembly of more than 85%.

### Example 8. Flap Endonuclease Mediated Nucleic Acid Assembly of 1.8 kb Fragment

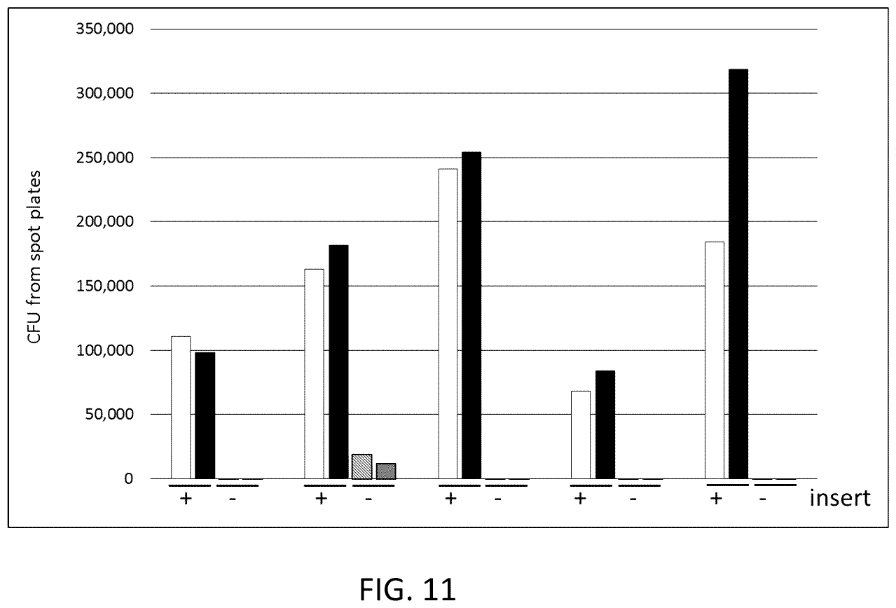

A flap endonuclease mediated nucleic acid assembly reaction was prepared similarly to Example 4. Flap endonuclease mediated nucleic acid assembly reactions were performed using enzyme concentrations of 1 U ExoIII, 0.2 U Phusion, 1 U Ampligase, and 0.32 U Fen1 with insert (white bars) and without insert (hashed bars, third bar from left). Flap endonuclease mediated nucleic acid assembly reactions were also tested using enzyme concentrations of 1 U ExoIII, 0.1 U Phusion, 1 U Ampligase, and 0.32 U Fen1 with insert (black bars) and without insert (hashed bars, fourth from left). Colony forming units from spot plates were then measured (Y-axis). Referring to FIG. 11, there was an increased number of CFUs in reactions comprising insert as compared to reactions without insert.

### Example 9. Flap Endonuclease Mediated Nucleic Acid Assembly of Two DNA Fragments

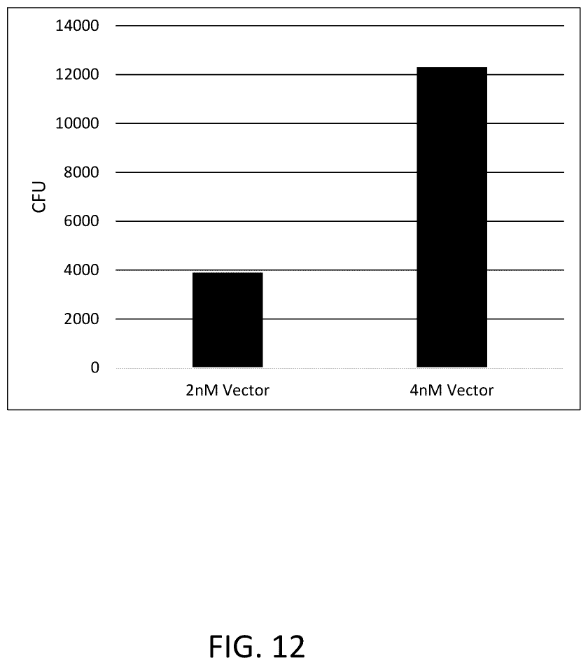

A flap endonuclease mediated nucleic acid assembly reaction was prepared similarly to Example 4. Different amounts of input DNA or linearized vector on colony forming units (CFU) were assayed. The amount of input DNA tested was 2 nM or 4 nM of linearized vector. Referring to FIG. 12, two fragments of DNA were assembled and there was a positive correlation with the amount of starting material to the amount of colony forming units.

### Example 10. Multi-piece DNA Assembly Into a DNA Vector

A flap endonuclease mediated nucleic acid assembly reaction was prepared similarly to Example 4. Experiments were performed using reaction concentrations of the reagents as seen in Table 14. The reactions were prepared on ice, and following addition of the various reagents, the reactions were incubated at 50° C. for 30 minutes. The reactions were then diluted 1:5 and transformed into E. coli.

TABLE 14
Method 1 Reaction Concentrations

| Reagent | 5 uL reaction | Final Concentration | |
|---|---|---|---|
| dNTP (10 mM) | 0.1 | .2 | mM |
| 10x Ampligase | 0.5 | 1X | |
| buffer | | | |
| ExoIII (100 U/uL) | 0.005 | 0.1 | U/uL |
| Phusion (2 U/uL) | 0.05 | 0.02 | U/uL |
| Ampligase (5 U/uL) | 0.1 | 0.1 | U/uL |
| Fen1 (32 U/uL) | 0.005 | 0.032 | U/uL |
| Vector DNA | | 20 | fmol |

| | | |
|---|---|---|
| Insert DNA | | 40 fmol/Insert |
| Water * | To 5 uL | |

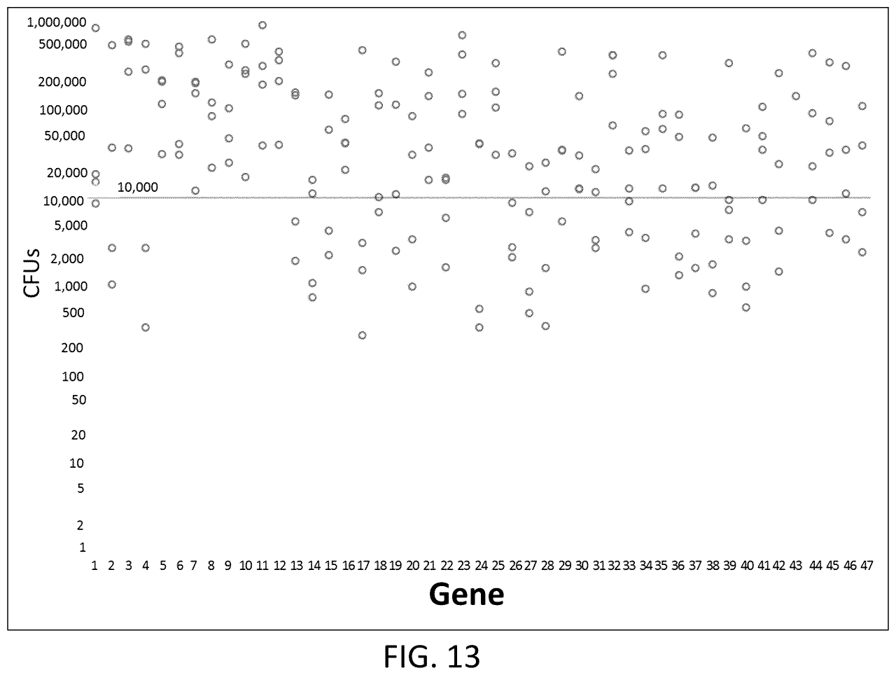

Colony forming units (CFUs) were then measured. As seen in FIG. 13, colony forming units (y-axis) was measured for the different genes tested (x-axis). From the data, the flap endonuclease mediated nucleic acid assembly reaction according to the reaction conditions described resulted in high CFUs and assembly fidelity rates.

### Example 11. Multi-piece DNA Assembly Into a DNA Vector Using High Amount of ExoIII

A flap endonuclease mediated nucleic acid assembly reaction was prepared similarly to Example 4 and Example 10. The concentration of ExoIII was increased 16 fold as compared to Example 10. The reaction concentrations are seen in Table 15. The reactions were prepared on ice, and following addition of the various reagents, the reactions were incubated at 65° C. for 30 minutes. The reactions were then diluted 1:5 and transformed into E. coli.

TABLE 15
Method 2 Reaction Concentrations

| Reagent | 5 uL reaction | Final Concentration | |
|---|---|---|---|
| dNTP (10 mM) | 0.1 | .2 | mM |
| 10x Ampligase | 0.5 | 1x | |
| buffer | | | |
| ExoIII 100 U/uL | 0.08 | 1.6 | U/uL |
| Phusion 2 U/uL | 0.05 | 0.02 | U/uL |
| Ampligase 5 U/uL | 0.1 | 0.1 | U/uL |
| Fen1 32 U/uL | 0.005 | 0.032 | U/uL |
| Vector DNA | | 20 | fmol |

| | | |
|---|---|---|
| Insert DNA | | 40 fmol/Insert |
| Water * | To 5 uL | |

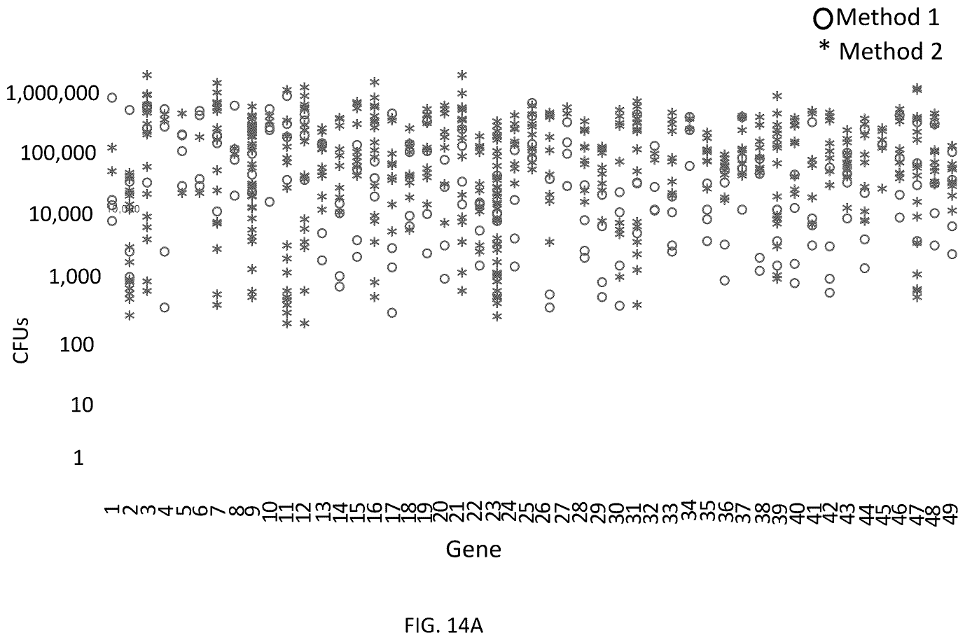

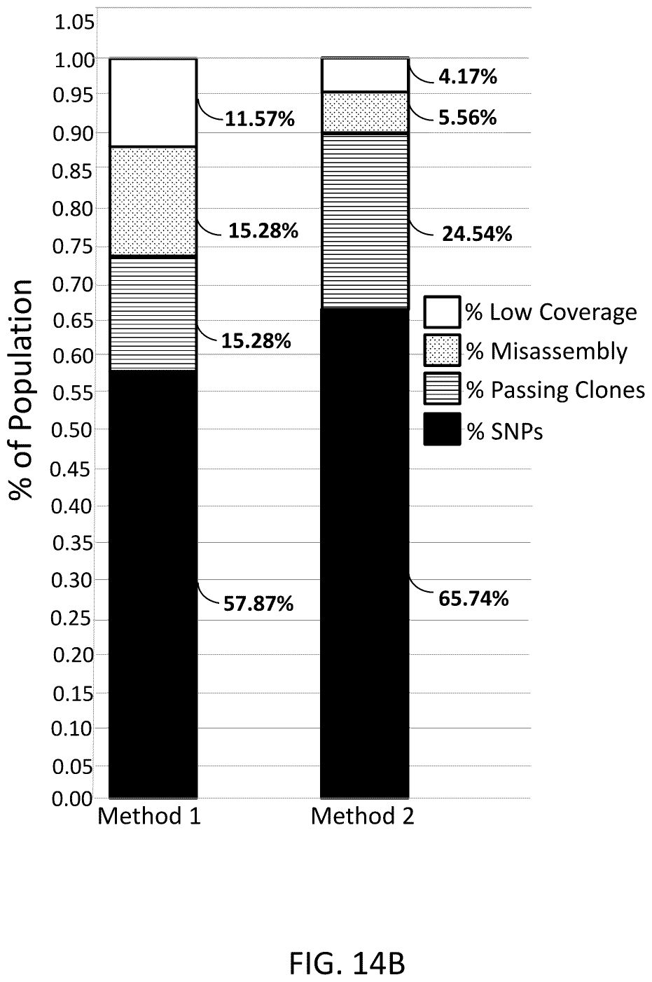

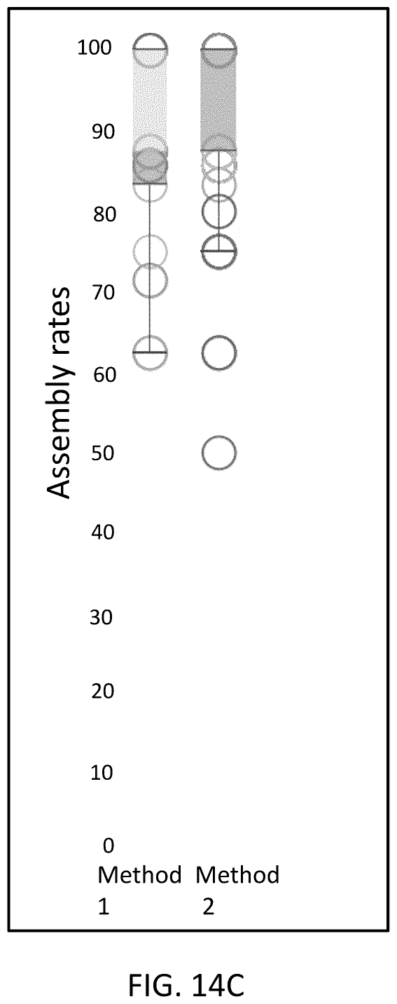

Colony forming units were then measured. As seen in FIG. 14A, colony forming units (y-axis) was measured for the different genes tested (x-axis) using reaction concentration according to Method 1 (Table 14) and reaction concentrations according to Method 2 (Table 15). Results from FIG. 14A show higher CFU and greater assembly fidelity using Method 2.

Next generation sequencing (NGS) was also performed. As seen in FIG. 14B, empty or low coverage (white bars), misassembly (dotted bars), passing clones (horizontal bars), and SNPs (black bars) were measured following flap endonuclease mediated nucleic acid assembly using reaction concentrations according to Method 1 (Table 14) and reaction concentrations according to Method 2 (Table 15). The NGS results showed similar results as FIG. 14A in that higher CFUs and improved assembly fidelity was observed using Method 2. Referring to FIG. 14C, assembly rate (y-axis) was compared using Method 1 and Method 2 (x-axis). Assembly fidelity rates were improved using Method 2 as compared to Method 1.

The example shows that the flap endonuclease mediated nucleic acid assembly reaction using the described reaction conditions resulted in higher colony forming units and improved assembly fidelity.

### Example 12: Flap Endonuclease Mediated Nucleic Acid Assembly Using a Bridged Assembly Method

A flap endonuclease mediated nucleic acid assembly reaction was prepared similarly to Example 4 and Example 10. The reaction conditions are seen in Table 14. In addition, 40 fmol of a DNA bridge was used.

The various samples included assembly with no bridge (negative control), fragments comprising a 40 base pair homology sequence (positive control), double stranded DNA bridge comprising a 40 base pair homology sequence to each fragment, and a double stranded DNA bridge comprising a 50 base pair homology sequence to each fragment. The reactions were prepared and transformed into E. coli. The number of correct assemblies and percentage of correct assemblies were then determined and are seen in Table 16.

TABLE 16
Bridged Assembly Method

| | | Correct | % Correct |
|---|---|---|---|
| Sample Description | Clones Tested | Assemblies | Assemblies |
| Negative Control (no | 12 | 0 | 0 |
| bridge) | | | |
| Positive Control (fragments | 12 | 12 | 100 |
| have 40 bp homology) | | | |
| dsDNA bridge with 40 bp | 12 | 12 | 100 |
| homology to each insert. | | | |
| dsDNA bridge with 50 bp | 12 | 12 | 100 |
| homology to each insert | | | |

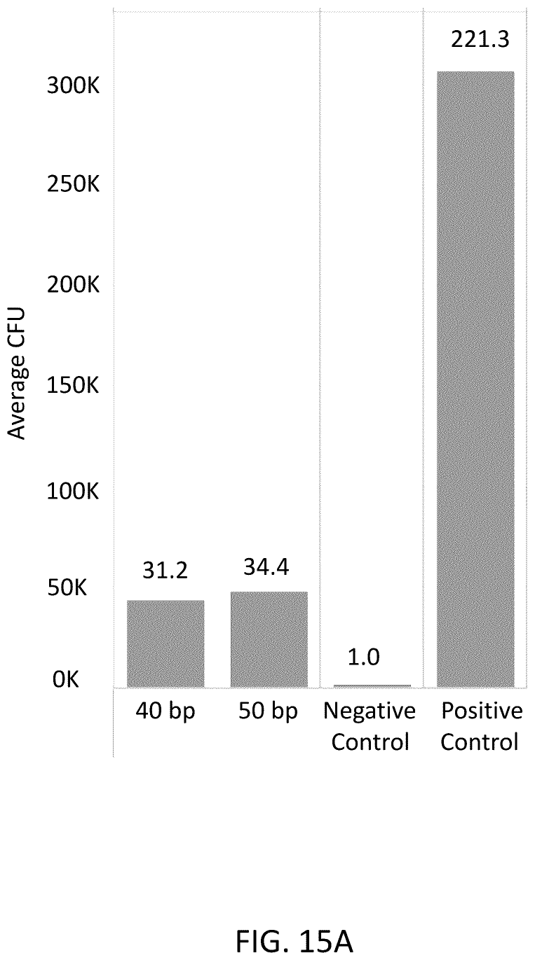

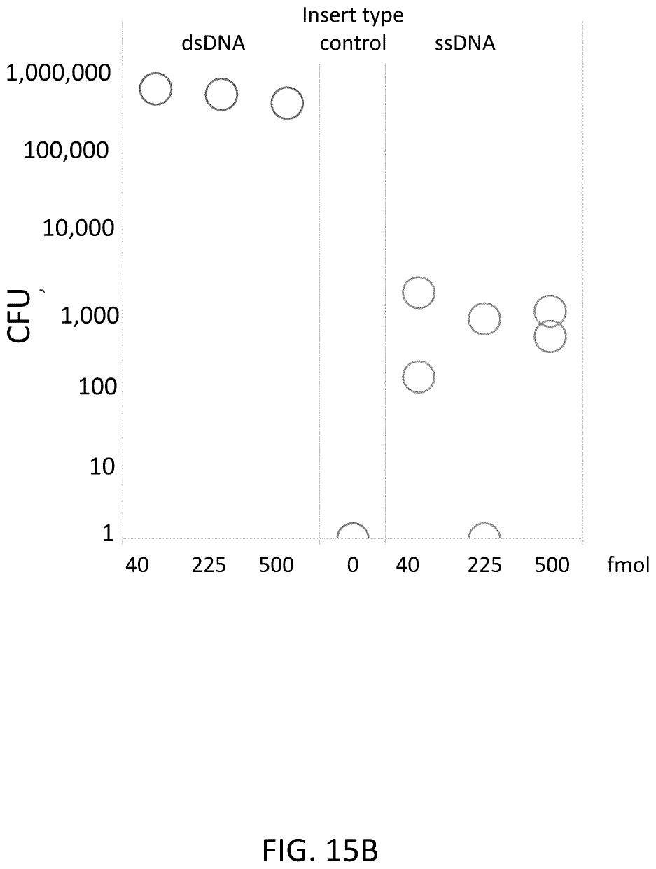

As seen in Table 16, flap endonuclease mediated nucleic acid assembly reaction using a double stranded DNA (dsDNA) bridge of 40 base pairs and 50 base pairs resulted in more than 90% correct assemblies. Further as seen in FIG. 15A, assembly using a dsDNA bridge of 40 base pairs and 50 base pairs resulted in higher levels of assembly as compared to the negative control.

CFUs using the bridged assembly method were measured. As seen in FIG. 15B, colony forming units from assembly with single stranded DNA (ssDNA) at various amount of insert (40 fmol, 225 fmol, or 500 fmol) was higher than the negative control (control). As a positive control, ssDNA was PCR amplified to create a double stranded DNA (dsDNA) and was assembled.

The data indicates that assembly of ssDNA by flap endonuclease mediated nucleic acid assembly using a bridge nucleic acid results in a higher percentage of correct assemblies and higher number of colony forming units.

### Example 13: Flap Endonuclease Mediated Nucleic Acid Assembly of Twelve Fragments

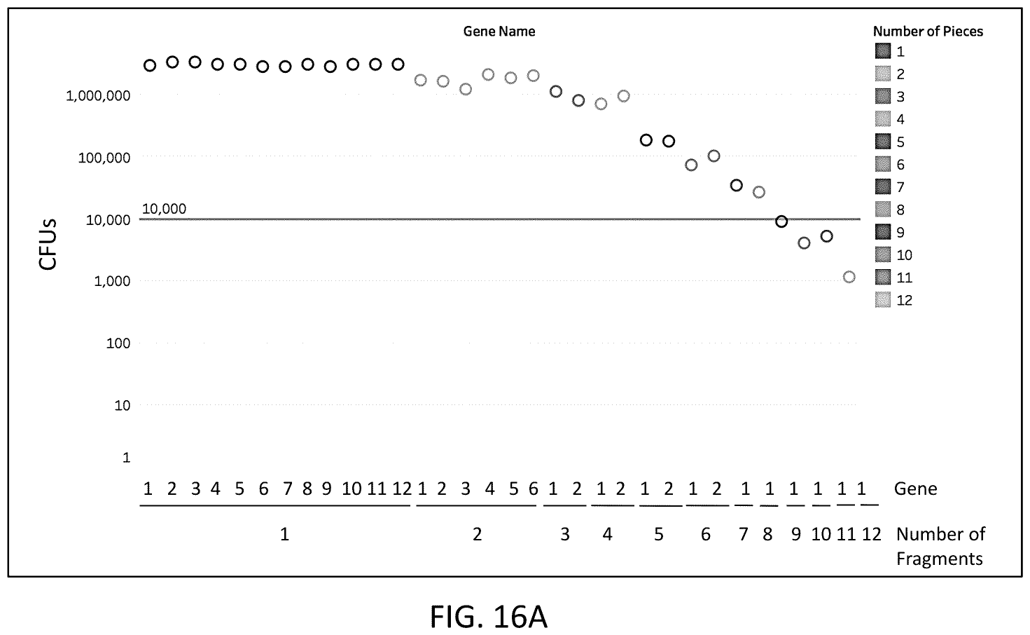

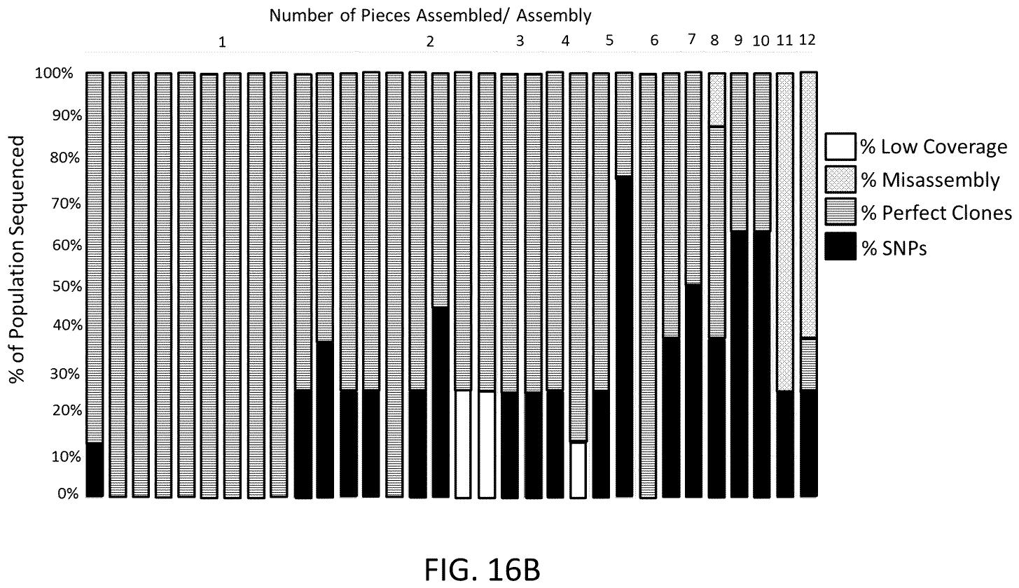

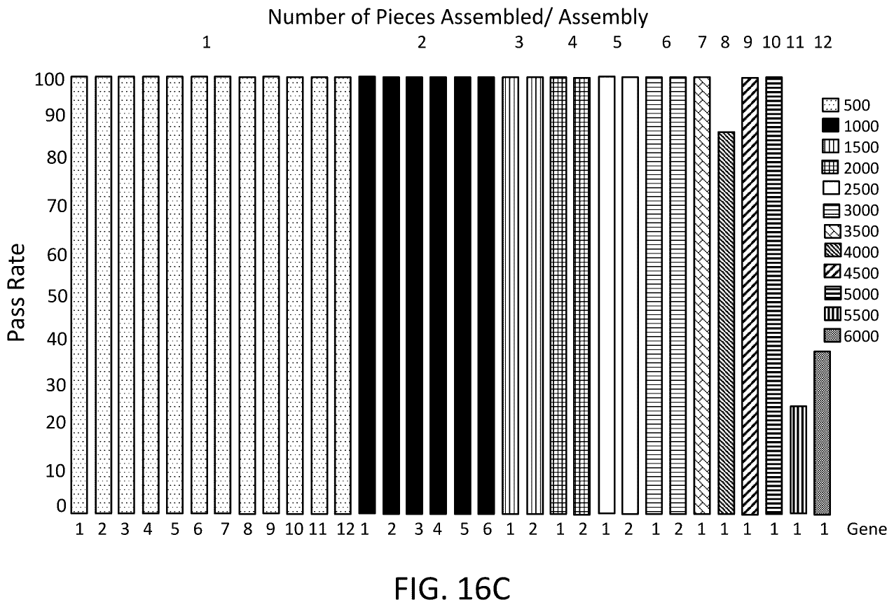

A flap endonuclease mediated nucleic acid assembly reaction was prepared similarly to Example 4 and Example 10. Multiple fragments were assembled into a vector including 1, 2, 3, 4, 5, 6, 7, 8, 9, 10, 11, and 12 fragments. Each DNA fragment was 500 base pairs. Following assembly reactions, the reactions were transformed intoE. coli and colony forming units were measured. FIG. 16A illustrates a graph of colony forming units for assembly of 1, 2, 3, 4, 5, 6, 7, 8, 9, 10, 11, and 12 fragments.

Next generation sequencing (NGS) was also performed. As seen in FIG. 16B, empty or low coverage (white bars), misassembly (dotted bars), passing clones (horizontal bars), and SNPs (black bars) were measured following flap endonuclease mediated nucleic acid assembly. Pass rates were also measured as seen in FIG. 16C. The data shows assembly of 12 fragments result in cloning success and high pass rates.

### Example 14: Flap Endonuclease Mediated Nucleic Acid Assembly Using Low Amount of Polymerase

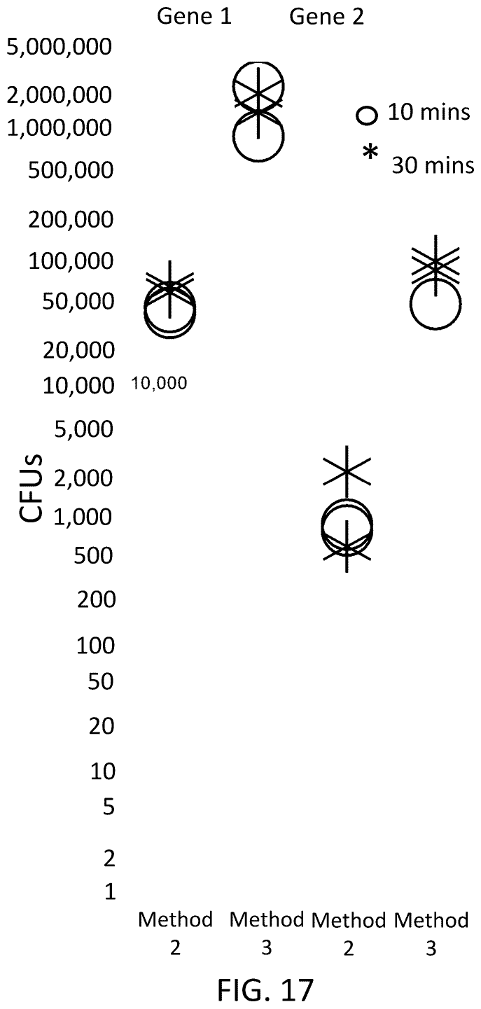

A flap endonuclease mediated nucleic acid assembly reaction was prepared similarly to Example 4 and Example 10. The concentration of Phusion polymerase was decreased 10 fold as compared to Example 10. The reaction concentrations are seen in Table 17. The reactions were prepared on ice, and following addition of the various reagents, the reactions were incubated at 65° C. for 10-30 minutes. The reactions comprising three fragments were cloned into a plasmid and transformed into E. coli. Colony forming units were measured following 10 minute incubation and 30 minute incubation as seen in FIG. 17. There was an increased number of CFUs using decreased Phusion polymerase according to the reaction concentrations in Table 17 as compared to the reaction concentrations described in Example 11 (Method 2). Similar CFUs were measured following 10 minute incubation or 30 minute incubation. Data shows that cloning efficiency was improved using a decreased amount of polymerase.

TABLE 17
Method 3 Reaction Conditions

| | 5 uL | Final | |
|---|---|---|---|
| Master Mix | reaction | Concentration | |
| dNTP | 0.1 | .4 | mM |
| 10x Ampligase buffer | 0.5 | 2x | |
| (Epicenter) | | | |
| ExoIII 100 U/uL | 0.08 | 1.6 | U/uL |
| (NEB) | | | |
| Phusion 2 | 0.005 | 0.002 | U/uL |
| U/uL (NEB) | | | |
| Ampligase 5 U/uL | 0.1 | 0.1 | U/uL |
| (Epicenter) | | | |
| Fent 32 U/uL (NEB) | 0.005 | 0.032 | U/uL |
| Water * | 1.695 | | |

### Example 15: Flap Endonuclease Mediated Nucleic Acid Assembly of Non-Clonal Fragments

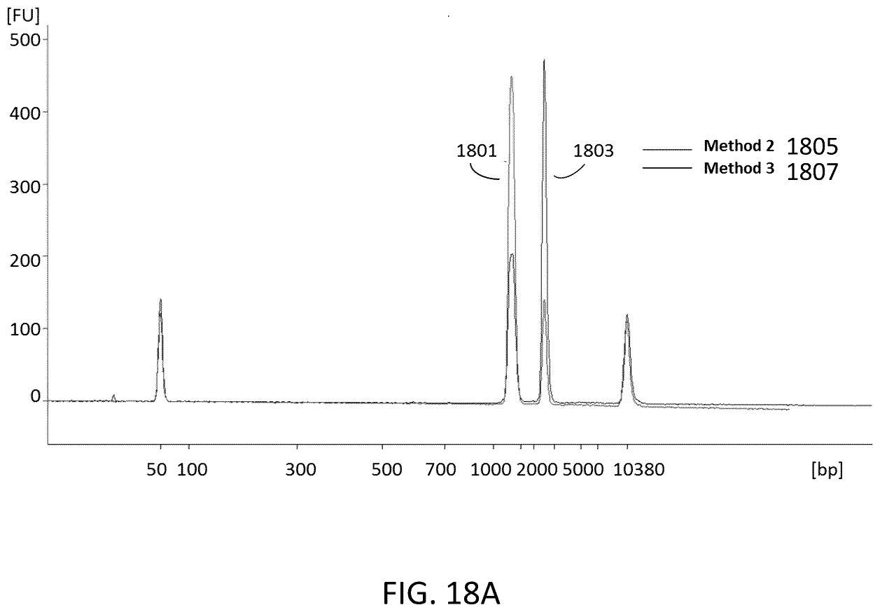

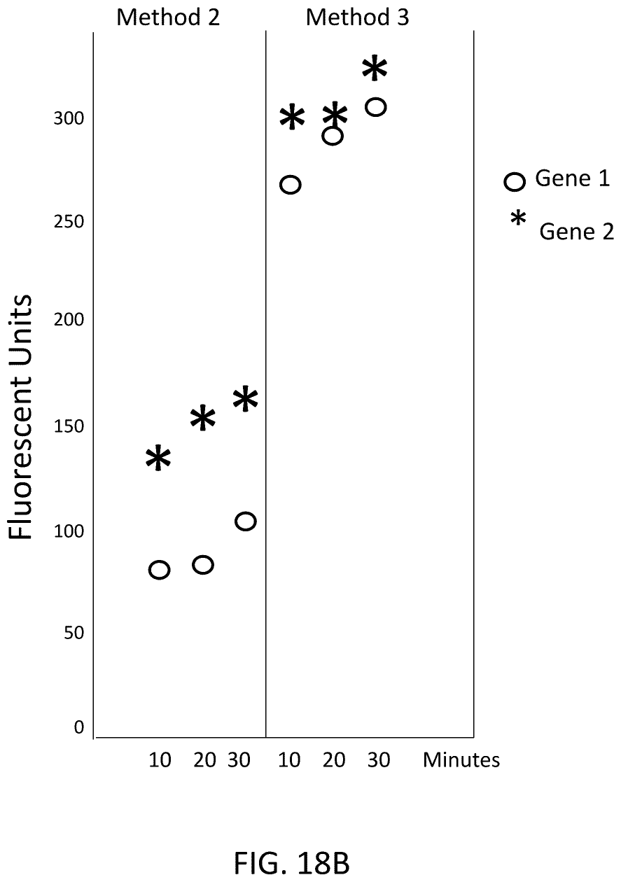

Reaction efficiency of assembly of non-clonal fragments was determined. Reactions were prepared for assembling 2 DNA fragments using Method 2 as seen in Table 15 and Method 3 as seen in Table 17. Reactions were then incubated at 65° C. for 10-30 minutes. Assembly PCR products were analyzed on a BioAnalyzer (FIG. 18A) with correctly assembled fragments being 3000 base pairs. Referring to FIG. 18A, 1801 is unassembled gene fragments and 1803 is assembled gene fragments using Method 2 (red line, 1805) and Method 3 (blue line, 1807).

Non-clonal assembly was also determined. Referring to FIG. 18B, fluorescent units (y-axis) was measured for 10 minutes, 20 minutes, or 30 minutes of incubation using Method 2 or Method 3 reaction conditions for Gene 1 and Gene 2. There was an increase in fluorescent units at 10 minutes, 20 minutes, or 30 minutes of incubation using Method 3 compared to Method 2. The data shows improved assembly of non-clonal fragments using Method 3.

### Example 16: Improved Assembly With Flap Endonuclease Mediated Nucleic Acid Assembly

Flap endonuclease mediated nucleic acid assembly using similar reaction conditions as described in Example 14 was compared to assembly by different comparators. Twelve 500 bp sequences were generated, and a series of constructs for assembly of one to ten DNA fragments into a vector were designed. DNA was assembled with homologous ends using the flap endonuclease mediated nucleic acid assembly method, Comparator 1 method, and Comparator 2 method. Comparator 1 assembly relies on homology at the ends of a construct that is quickly introduced by PCR or through DNA synthesis. Each fragment in Comparator 1 assembly requires a different pair of PCR primers. Comparator 2 method is an assembly method for nucleic acid fragments with varied overlap regions. Effects of multiple homology lengths and incubation times were determined.

Flap endonuclease mediated nucleic acid assembly was performed at 65° C. At 65° C. most secondary structures were eliminated and assembly fidelity was greatly improved (data not shown).

The efficiency and the accuracy of assembly using the flap endonuclease mediated nucleic acid assembly method, Comparator 1 method, and Comparator 2 method were determined. Efficiency was determined by colony forming units (CFUs), and accuracy was determined by next generation sequencing (NGS).

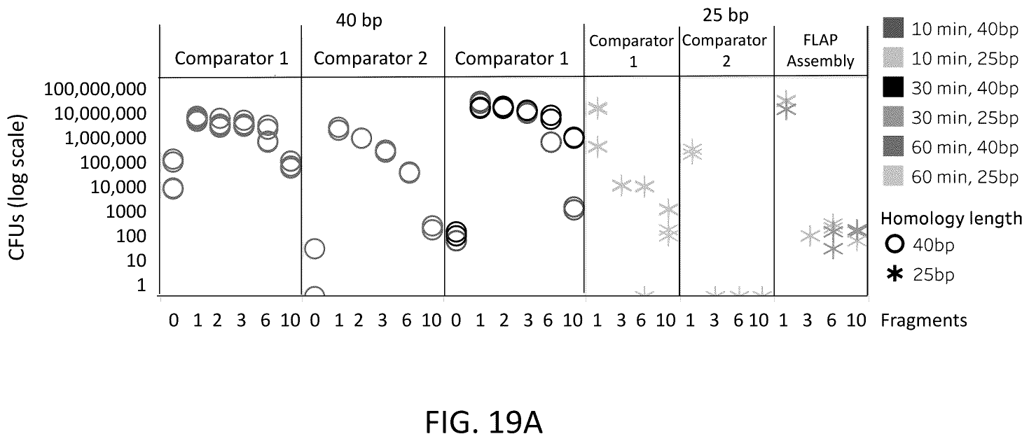

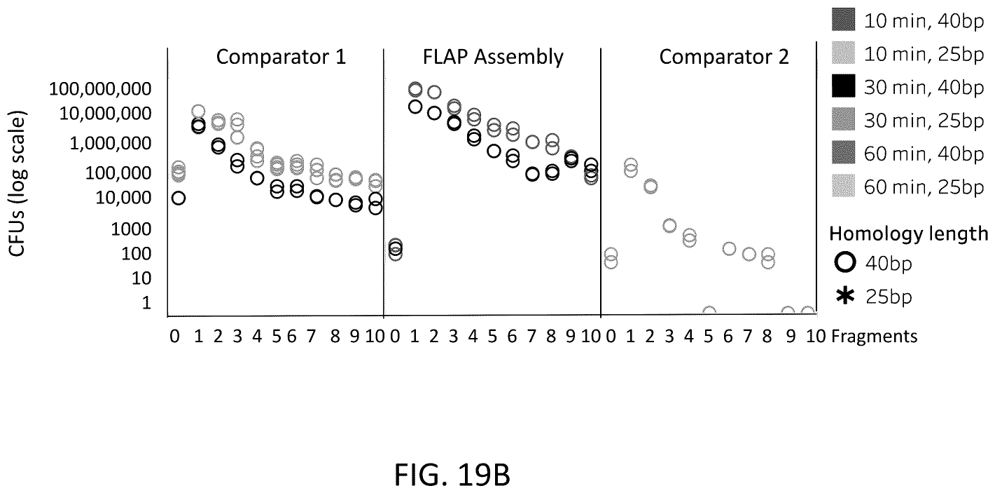

Efficiency using the flap endonuclease mediated nucleic acid assembly method, Comparator 1 method, and Comparator 2 method was determined for double stranded DNA (dsDNA) non-clonal fragments comprising adaptor sequences. The adaptor sequences acted as universal primer pairs. dsDNA comprising homology ends was generated by amplifying dsDNA fragments with primers specific for each construct (“adaptors-off”). Each fragment contained 40 or 25 bp of overlapping homology to their intended destination. One to ten dsDNA fragments were assembled into a linearized plasmid and transformed the reaction into E. coli. For one fragment assemblies, flap endonuclease mediated nucleic acid assembly method, Comparator 1 method, and Comparator 2 method resulted in robust colony forming units. The flap endonuclease mediated nucleic acid assembly method resulted in higher colony forming units regardless of reaction time, homology lengths, and number of pieces assembled (FIG. 19A). Flap endonuclease mediated nucleic acid assembly method resulted in colony forming units higher than background for fragment assemblies for 6 or more fragments (FIG. 19A). When the number of fragments was increased, the flap endonuclease mediated nucleic acid assembly method was more efficient with a 30 minute reaction as compared to a 10 minute reaction (FIG. 19A). A 25 bp homology length was also tested using the flap endonuclease mediated nucleic acid assembly method, Comparator 1 method, and Comparator 2 method, and for all three methods was unsuccessful for assemblies of two or more fragments.

Efficiency for DNA with buried homology sequences (“adaptors-on”) was determined using the flap endonuclease mediated nucleic acid assembly method, Comparator 1 method, and Comparator 2 method. Forty base pair homologies buried ˜23 bp from the end of the DNA fragment were designed. All methods gave CFUs significantly higher than their respective backgrounds (FIG. 19B). Referring to FIG. 19B, one fragment flap endonuclease mediated nucleic acid assembly with a ten minute incubation resulted in 9-fold more colonies than assemblies using Comparator 1 method and 568-fold more colonies than assemblies using Comparator 2. When the number of inserts was increased, Comparator 1 method and Comparator 2 method failed to give colonies over background levels at four fragments (FIG. 19B). Flap endonuclease mediated nucleic acid assembly of ten fragments into a vector resulted in a 642-fold increase over background (FIG. 19B). Assembly of DNA with end homology sequences and buried homology sequences resulted in more efficient assemblies using the flap endonuclease mediated nucleic acid assembly as compared to assembly using Comparator 1 method or Comparator 2 method.

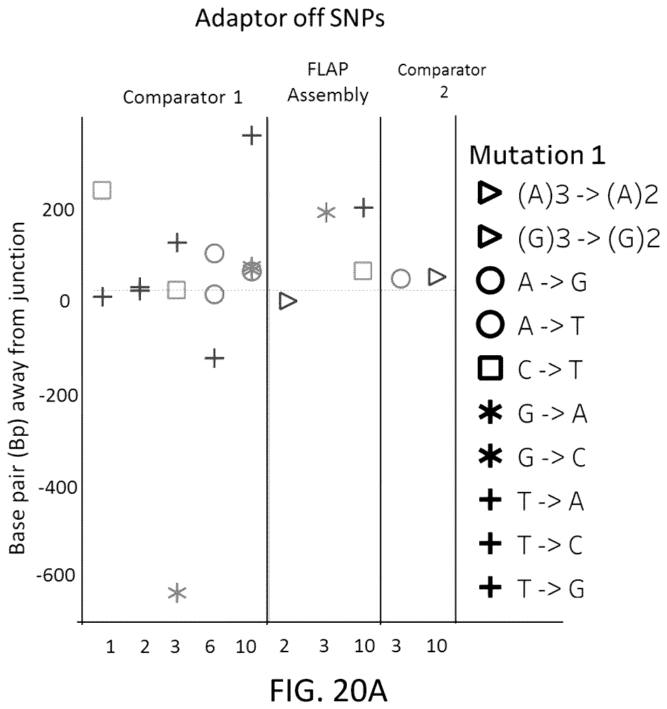

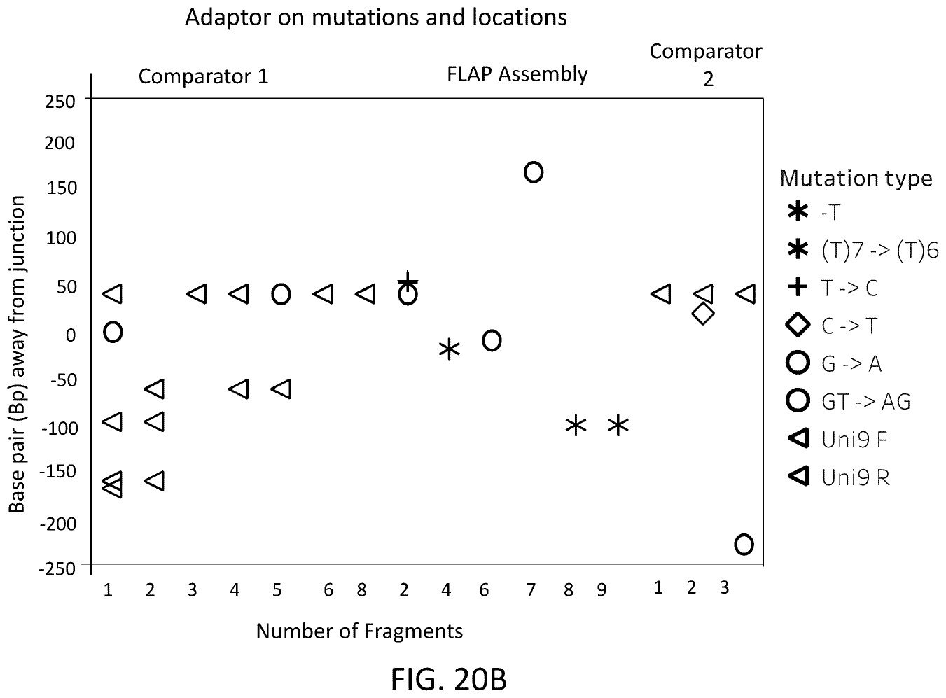

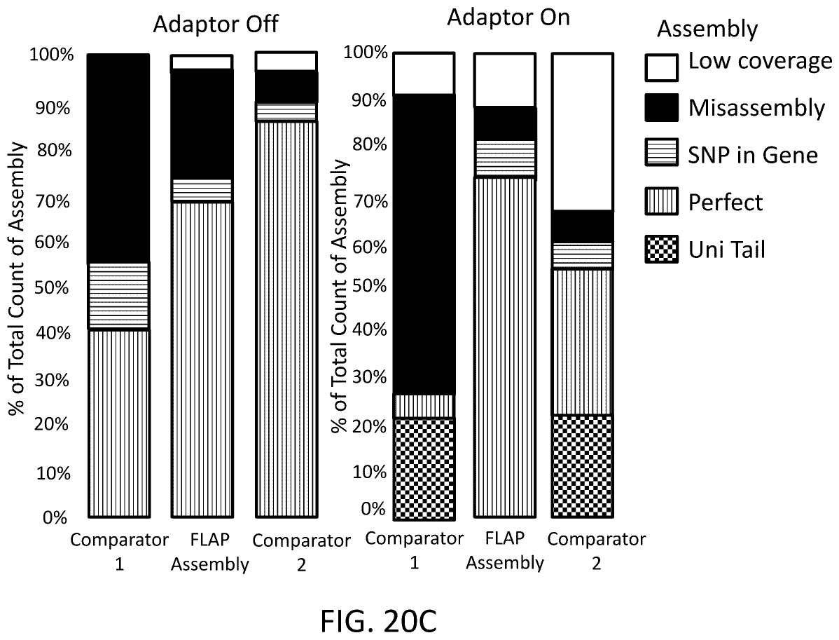

Next generation sequencing was performed using plasmids isolated from 8 colonies per reaction. The flap endonuclease mediated nucleic acid assembly and Comparator 2 method resulted in 84% and 86% correct assembly rates, respectively. Each method showed 8% of samples misassembled. Assemblies using Comparator 1 method resulted in 10% misassembly and a 25% SNP rate, resulting in an overall correct assembly rate of 65%. Analyzing the pass and failure rates dependent on the number of inserts assembled, Comparator 1 method resulted in a loss of fidelity at 10 inserts. Most errors were clustered within 25 bp of the fragment junctions for Comparator 1 assembly and Comparator 2 assembly (FIGS. 20A-20C).

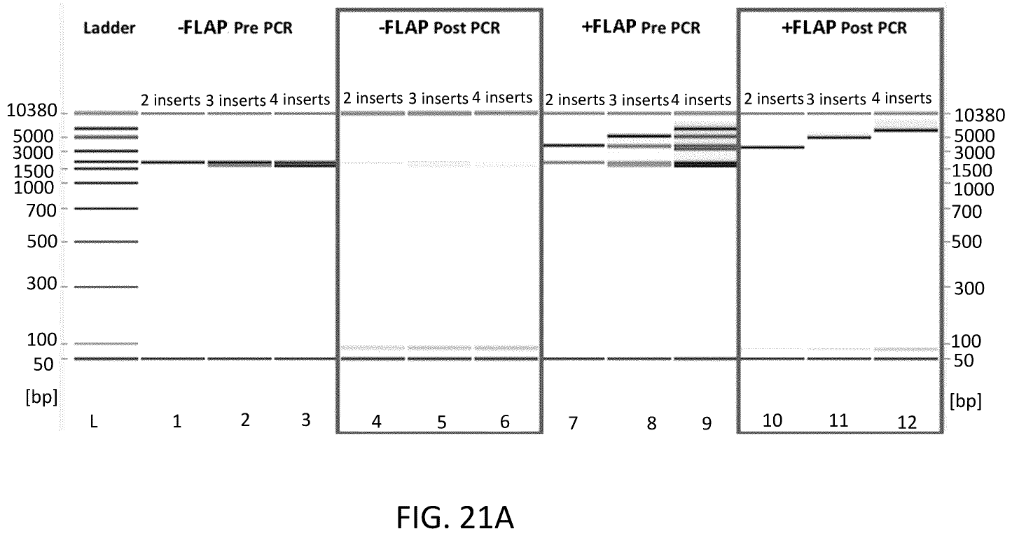

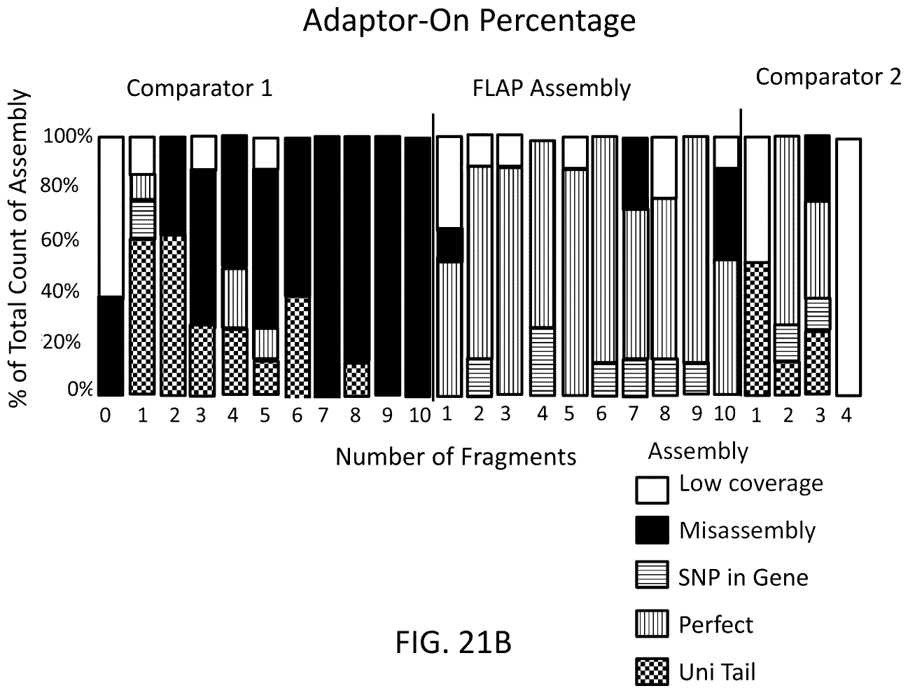

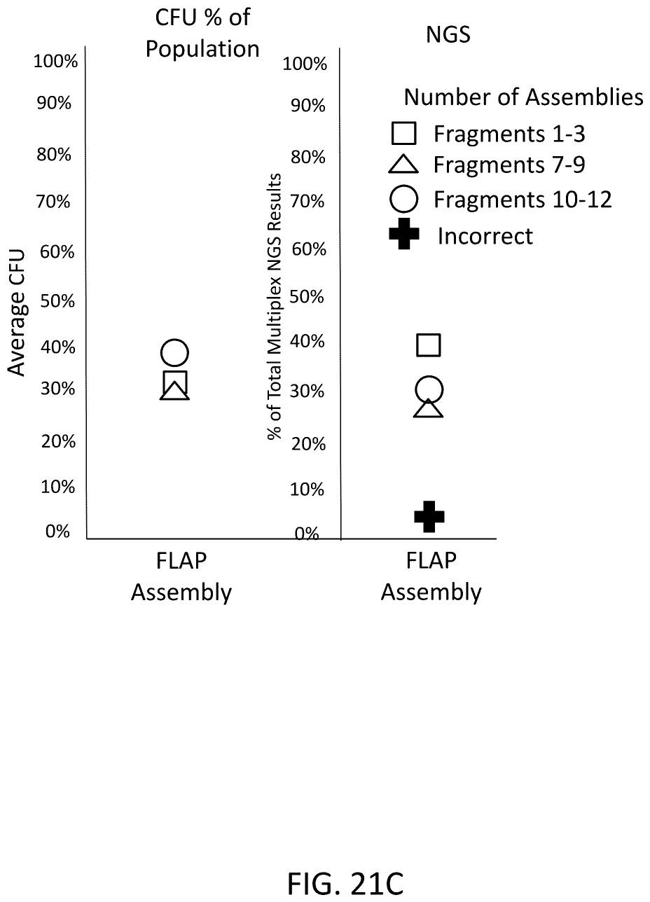

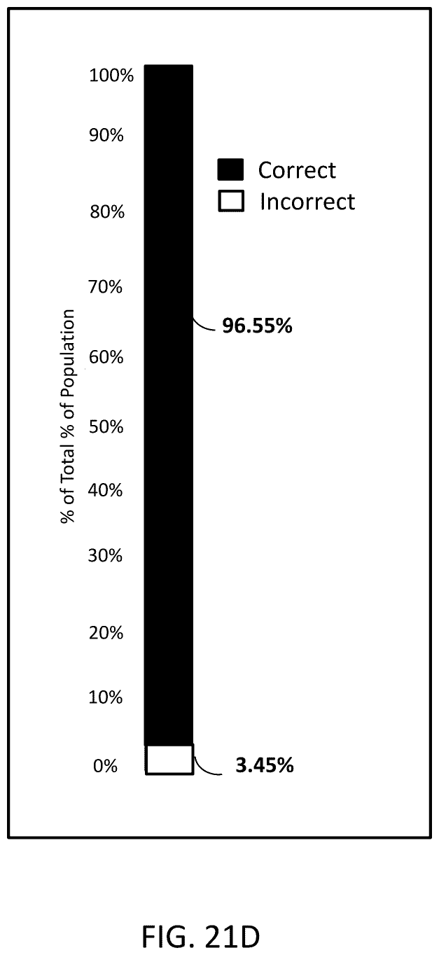

Next generation sequencing was used to determine the presence of the 23 bp adaptor. Flap endonuclease mediated nucleic acid assembly resulted in higher correct assembly rates than Comparator 1 and Comparator 2 methods. Across all assembly reactions the average correct assembly rate was 72% for flap endonuclease mediated nucleic acid assembly reactions, compared to 4.5% for Comparator 1 assemblies and 31% for Comparator 2 assemblies. The adaptor sequence was never present in the flap endonuclease mediated nucleic acid assembly samples. In contrast, for full length constructs, 59% of Comparator 1 assemblies and 23% of Comparator 2 assemblies contain partial or full-length adaptor sequences. Constructs assembled by Comparator 1 assembly were more likely to misassembly, with an overall misassembly rate of 63% as compared to 7% for flap endonuclease mediated nucleic acid assembly and 6% for Comparator 2. The misassembly rates increased with the number of fragments in the Comparator 2 assemblies. Further, Comparator 1 assembly had high CFUs in the negative control reactions. Sequencing of the 24 Comparator 1 negative control samples (vector with no inserts) showed that each construct was the vector recombined to itself at various regions of the backbone. See FIG. 20C and FIG. 21B. FIG. 20C shows a graph of low coverage (white bars), misassembly (black bars), SNP in gene (horizontal hatched bars), perfect assembly (vertical hatched bars), and universal (uni) tail (checkered bars). Table 18 shows the data from FIG. 20C. Flap endonuclease mediated nucleic acid assembly resulted in more efficient and accurate assembly as compared to different assembly methods. FIG. 21B shows a graph of low coverage (white bars), misassembly (black bars), SNP in gene (horizontal hatched bars), perfect assembly (vertical hatched bars), and universal (uni) tail (checkered bars).

TABLE 18
Sequencing Data

| | Comparator 1 | | FLAP Assembly | | Comparator 2 | |
|---|---|---|---|---|---|---|
| | Adaptor | Adaptor | Adaptor | Adaptor | Adaptor | Adaptor |
| | Off | On | Off | On | Off | On |
| Low | NA | 9.09% | 3.57% | 11.25% | 4.17% | 34.38% |
| Coverage | | | | | | |
| Misassembly | 45.45% | 63.64% | 23.21% | 7.5% | 6.25% | 6.25% |
| SNP in Gene | 13.64% | NA | 5.36% | 8.75% | 4.17% | 6.25% |
| Perfect | 40.91% | 4.55% | 67.86% | 75.20% | 85.42% | 31.25% |
| Uni Tail | NA | 21.59% | NA | NA | NA | 21.88% |

### Example 17: Improved Flexibility With Flap Endonuclease Mediated Nucleic Acid Assembly

### In Vitro Seamless Assembly

Assembly of DNA fragments was determined using flap endonuclease mediated nucleic acid assembly. Flap endonuclease mediated nucleic acid assembly using similar reaction conditions as described in Example 14 was used to assemble 2, 3, and 4 linear dsDNA fragments together. In reactions comprising the flap endonuclease mediated nucleic acid assembly enzymatic cocktail, unincorporated starting material, partial and full-length constructs were detected (FIG. 21A, lanes 1-3, 7-9). In the absence of the flap endonuclease mediated nucleic acid assembly enzymatic cocktail after incubation at 65° C. for 30 minutes, there was starting material but not fully assembled constructs. To enrich for full length constructs, the reactions were PCR amplified using terminal primer sites specific for the full-length constructs. In the absence of the flap endonuclease mediated nucleic acid assembly enzymatic cocktail, no full length constructs were observed after PCR amplification (FIG. 21A, lanes 4-6). Products at the proper size for full length constructs were observed (FIG. 21A, lanes 10-12). Flap endonuclease mediated nucleic acid assembly resulted in properly assembled fragments in vitro.

### One Pot Combinatorial Assembly

Specificity of the flap endonuclease mediated nucleic acid assembly was determined. Nine linear DNA fragments comprising the same universal primer tails were used. Forty bp homology sites directed to directionally assemble three different 3-piece constructs into the same vector were designed. The individual cloning efficiencies for each construct to screen for toxicity and establish an assembly baseline were determined. All nine DNA fragments and the destination vector were then used in a single in vitro assembly reaction. After cloning the reaction mixture into E. coli, 192 colonies were picked for mini-prep followed by NGS analysis. Referring to FIGS. 21A, 21C, and 21D, the expected median distributions, based on cloning individual constructs, was 31%±3% for each construct.

Referring to FIG. 21C, the average CFU was determined for assemblies comprising fragments 1-3 (squares), fragments 7-9 (triangles), fragments 10-12 (circles), and incorrect assembly (cross). After the combinatorial assembly the spread was increased to ±5% (FIG. 21C). Of the constructs tested, 96.5% contained properly assembled constructs (FIG. 21D). Failed assemblies consisted of full length genes of Fragments 7-8, Fragments 7-12, Fragments 9-12. Flap endonuclease mediated nucleic acid assembly resulted in specific, directional assembly.

### Example 18. Cloning of Amplified Oligonucleotide Populations

Multiple fragments were assembled into genes. Cloning of amplified oligonucleotide populations was performed similar to Example 17. Overlap extension PCR was performed to generate double stranded DNA (dsDNA) fragments of more than 200 base pairs to assemble into a vector. Reactions were performed at 55° C. for 30 minutes and prepared according to a flap endonuclease mediated nucleic acid assembly method (Method 4) as seen in Table 19.

TABLE 19
Reaction Conditions (Method 4)

| | | Final | |
|---|---|---|---|
| | 50 uL | Concentration | |
| Master Mix | reaction | (for 2x MM) | |
| ExoIII 100 U/uL | 0.05 | 0.2 | U/uL |
| Phusion 2 U/uL | 0.05 | 0.004 | U/uL |
| Fen1 32 U/uL | 0.05 | 0.064 | U/uL |
| dNTP | 1 | .4 | mM |
| Ampligase | 1 | 0.2 | U/uL |

| | | |
|---|---|---|
| 5 U/uL | | |
| 10x Ampligase | 5 | 2x |
| buffer | | |
| Water * | | |

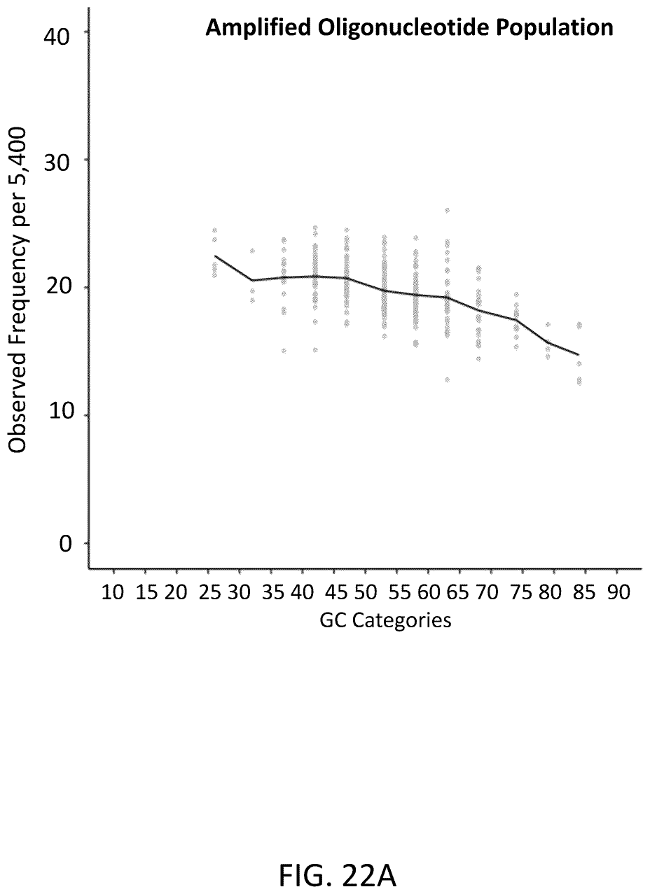

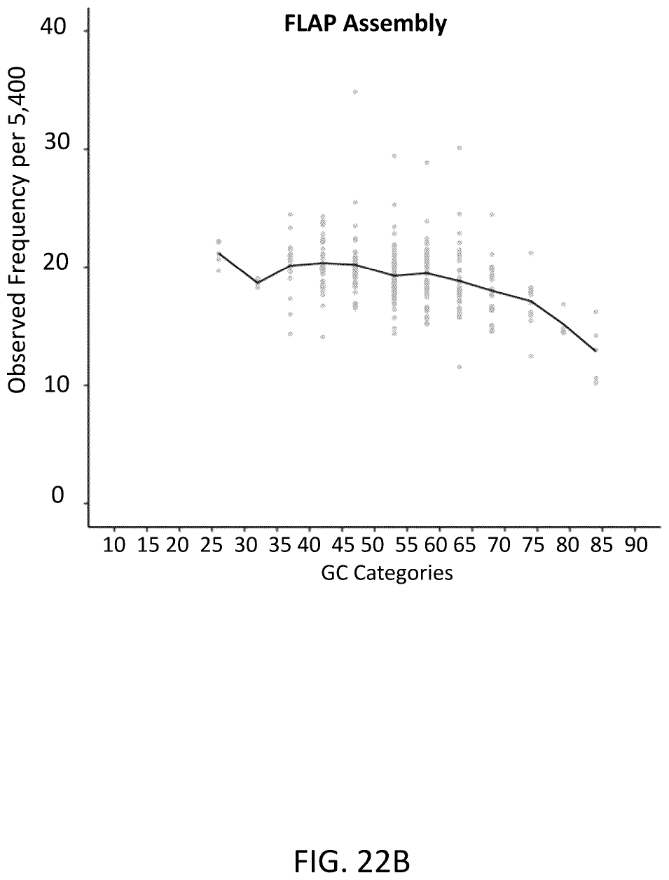

Next generation sequencing (NGS) was performed on the amplified oligonucleotide population prior to assembly (FIG. 22A) and following the flap endonuclease mediated nucleic acid assembly (FIG. 22B). FIGS. 22A-22B show that following flap endonuclease mediated nucleic acid assembly, there was no drop out of sequences and that samples were assembled without introducing significant sample bias as compared to the amplified oligonucleotide population prior to assembly.

### Example 19. Multiplexed Gene Assembly

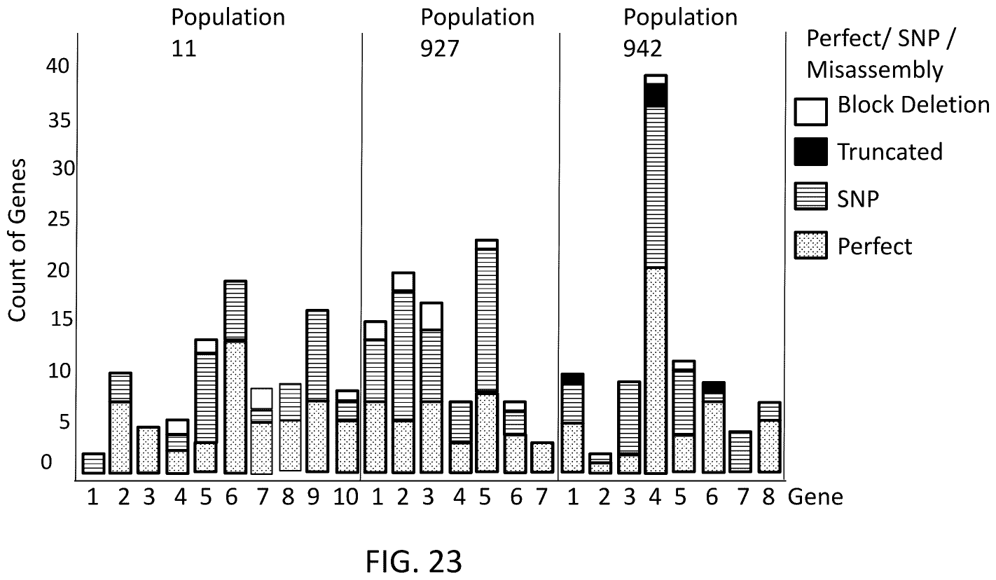

Multiplexed gene assembly was performed using a flap endonuclease mediated nucleic acid assembly method. Multiple sequences were assembled into 1 well. The sequences comprised two double stranded DNA (dsDNA) parts that are generated from overlap extension PCR. The reactions were prepared according to Method 4 as described in Table 19 and performed at 55° C. for 30 minutes. Samples were assembled and then cloned in a vector. Three populations were assembled: Population 11, Population 927, and Population 942. Each population comprised 96 individual clones that were Sanger sequenced to determine which gene was present. The quality of the starting DNA material was determined by measuring perfect sequences, sequences with SNPS, truncated material, or sequences with block deletions. As seen in FIG. 23, the accuracy of assembly was perfect (no chimeric genes) and uniformity/distribution was good.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-77. (canceled)

78. A method for nucleic acid assembly, comprising:
providing a first double stranded nucleic acid;
providing a second double stranded nucleic acid; and
mixing the first double stranded nucleic acid and the second double stranded nucleic acid with a reaction mixture comprising: a nucleic acid bridge, an exonuclease, an endonuclease, and a polymerase.

79. The method of claim 78, wherein the nucleic acid bridge comprises a first homology sequence and a second homology sequence, wherein the first homology sequence is homologous to a portion of the first double stranded nucleic acid, and the second homology sequence is homologous to a portion the second double stranded nucleic acid.

80. The method of claim 79, wherein the first homology sequence has a length that is 50 base pairs or less.

81. The method of claim 79, wherein the second homology sequence has a length that is about 40 base pairs to about 50 base pairs.

82. The method of claim 79, wherein the double stranded nucleic acid bridge further comprises a first universal primer binding sequence and a second universal primer binding sequence.

83. The method of claim 82, wherein the first homology sequence, the second homology sequence, or both, is between the first universal primer binding sequence and the second universal primer binding sequence.

84. The method of claim 78, wherein the nucleic acid bridge is double stranded.

85. The method of claim 78, wherein the endonuclease is a flap endonuclease.

86. A method for nucleic acid assembly, comprising:
providing a first double stranded nucleic acid comprising in 5′ to 3′ order: a first 5′ flanking adapter sequence, a first homology sequence, and a first 3′ flanking adapter sequence;
providing a second double stranded nucleic acid comprising in 5′ to 3′ order: a second 5′ flanking adapter sequence, a second homology sequence, and a second 3′ flanking adapter sequence; and
mixing the first double stranded nucleic acid and the second double stranded nucleic acid with a reaction mixture comprising at least one enzyme comprising 3′ or 5′ exonuclease activity, and a polymerase;
wherein the at least one enzyme comprising 3′ or 5′ exonuclease activity removes the first 5′ flanking adapter sequence, the second 5′ flanking adapter sequence, the first 3′ flanking adapter sequence, the second 3′ flanking adapter sequence, or a combination thereof.

87. The method of claim 86, wherein the polymerase comprises 5′ to 3′ polymerase activity.

88. The method of claim 86, wherein the reaction mixture comprises a concentration of the at least one enzyme comprising 3′ or 5′ exonuclease activity of about 0.1 U to about 10 U.

89. The method of claim 88, wherein the reaction mixture further comprises a ligase at a concentration less than or equal to 8.0 U.

90. The method of claim 86, wherein the first double stranded nucleic acid comprises an insertion sequence between the first homology sequence and the first 3′ flanking adapter sequence.

91. The method of claim 86, wherein the at least one enzyme comprising 3′ or 5′ exonuclease activity is exonuclease III.

92. A method for nucleic acid assembly, comprising:
receiving, at a processor, nucleic acid sequence data;
generating, at the processor, instructions for synthesis of a nucleic acid, based on the nucleic acid sequence data;
transmitting instructions from the processor to a material deposition device comprising a synthesis surface and a collection chamber; and
inducing the material deposition device to synthesize the nucleic acid, wherein synthesizing the nucleic acid includes:
generating a plurality of polynucleotides on the synthesis surface;
transferring the plurality of nucleotides from the synthesis surface to the collection chamber; and
mixing the plurality of polynucleotides with a reaction mixture comprising an exonuclease, a flap endonuclease, and a polymerase.

93. The method of claim 92, wherein synthesizing the nucleic acid further includes annealing the plurality of polynucleotides in an order based on a complementary sequence between adjacent polynucleotides.

94. The method of claim 92, wherein the synthesis surface comprises a plurality of discrete loci for synthesis.

95. The method of claim 92, wherein the collection chamber may comprise a top surface and bottom surface, wherein the plurality of polynucleotides are sandwiched between the top surface and the bottom surface during the assembly reaction.

96. The method of claim 92, wherein the collection chamber comprises a well or a channel, and the plurality of polynucleotides are within the well or the channel while the nucleic acid is synthesized.

97. The method of claim 92, wherein the endonuclease is a flap endonuclease.


SEQUENCE LISTING (4 sequences)

SEQ ID NO: 1 - DNA, 62 bp, synthetic construct
  modified_base 51..52: mod_base = OTHER; note = Thymidine-succinyl hexamide CED phosphoramidite
  source 1..62: mol_type = other DNA
agacaatcaaccatttggggtggacagccttgacctctagacttcggcattttttttttttt

SEQ ID NO: 2 - DNA, 112 bp, synthetic construct
  modified_base 101..102: mod_base = OTHER; note = Thymidine-succinyl hexamide CED phosphoramidite
  source 1..112: mol_type = other DNA
cgggatccttatcgtcatcgtcgtacagatcccgacccatttgctgtccaccagtcatgctagccataccatgatgatgatgatgatgagaaccccgcattttttttttttt

SEQ ID NO: 3 - DNA, 19 bp, synthetic construct
  source 1..19: mol_type = other DNA
atgcggggttctcatcatc

SEQ ID NO: 4 - DNA, 20 bp, synthetic construct
  source 1..20: mol_type = other DNA
cgggatccttatcgtcatcg